SEQUENCE LISTING (62 sequences)

SEQ ID NO: 1 - RNA, 2883 nt, Cannabis sativa
  source 1..2883: mol_type = mRNA
atggcggaagccatagtcagtttcgtgatcggaagactcggagacttactcataaacgaatctaatttcttgtacggaatccgagaccaagttgaggatgcaaaatctgagctccaacgaatcaaatgctttctcgaagatgccgatgctcgcgccagacacggagacaagtctcttcgtcttcatgtagctgaaatcggtgagtatgcttatgatttggaagatgttatcaccaattttgtattgaaagaatcttctagggttagtaatagtggtagtagcagtagtagtagtagtagaatgttctcttgtggtaatttcatcgaatttcacaaggttggatcgagaattgagaagatttcagctagggtttctaagtcaagattgagttttcaatcttatgggttggttcaatctaggggaaatgatgaagctgctactaatttttcaactaggttagaatttaggcgaactttttctcataatattgattctgattttgttgggtttgaagaaaatattaatgaattggttgagcatttgaagaaaaaagggagaaatttgcatagtgttgtgtcgatttgcggtatgggtggtcttggaaaaactactcttgctagacaggtttactatcatggtgaaattaggccttattttgattgttttgcttgggcttctgtatctcaaaaatgtcaacctagggatgtttgggaagggattttgattaggttgacatctcctgcatctgaaaggagaagagagattaaagaaatgaaagacgatgaaatcgctaaggctttgttcaaagtttgtactgaaaaaaggtgtcttgttgttcttgatgatatttggagttcttcgacatgggattgcctcaagcttgctttccctaatgttggatcagacagtaaaataatgcttaccactcgtaatagagaggtcgctttccatgcagatcgaaatagcattcttcatgaacccaggtgctttaacaaaaatgaaacttgggagttgtttatgatgaaaactcattttggaacagatgatatcgattcagaggattacaagaggaagaaaaagttatcagaggagatgcttgaatattgtggtggtttgccattagccattacagtgctcggaggacttctatctcgtaaaccaactgcagatgagtgggagacattgcataagaatattaagacatgtataaggaaaggaaaaataaatgagcaagaagactctaactttggtgtctcttgggtgttgggtttaagttacgacgagctaccatactatttaaagccatgttttttgcatttagcttattttccggaagattttgaaataagggctagagatttgtgcctagtatggttggcagaaggcttcgtatcgaaggaggagagagcatatgagtgcttaagtgaattggttgaaaggtgcataattcaagtagcagagtggagctcaactggcacaattaaaacatgtcgtattcatgatcttatgcgggatttatgcttagtaaaggctgaggaggaaggttttttgcagcatgctgatttacgcaatcatcacgaggtaacaaatttttcctcttcctctgttggagctgcaacaaatttagagccaattaataaggttcgtagacttgcgatctacttgaacaacaccagtgttgatgaattggtttctttaataagacataaggatggttgtcttaggtctcttatatgcttcaattcagaaccagatacatcctatgagcaagtgatgaagcccccgttcaatcaattccatttgctgagagttttgaagtttgagaattatactcacggtcatgttggaaagttgcctaaagaaattgggaatcttatccacttaaggctatttagtctcaaggatagttatgtggaaatatttccatcatctgtaggcaatttgagatgtctgcaaattttagatttgagagttaaaaacctgtatatcgacaaacaacttcaatcttcaaaattaccacctgtcctctggaaattggagcagttgagacatttgtatctgcctcaaaagtatttgacagaagctgagagaagcaagtttacagttactcacaatgagaatattttgcggttgggtaatctaaccaatttgcaaacattggtgaatgtttcggttaattatcattacttgaatgatttggagaaactaaccaatcttaccaaattaaaaataaatttggggtcttgcttccggggcacaagaattaaatttatcaatcttagatctttatccatttgttatgatgacatcatcatcgagagtgaagagtacggtacaataagacgaggagttgaattctcccaccatgagataatacttcttctactgagctgtcctcaacttcataaactgcatctgcaagtttcaatggtgaagttaccagaagaaaatcagttttcttcaaatctcatcaagctaacgctgcttcgaacttgtctcaaggatgatccaatggcaacattagaaaagctcccgcatttaaggattctcctcctcgatcacaatgccttcctcggacagaaaatgaattgcaccagtaaaggttttcctcgactcgagtctctatccatttgttatctttacgatttcatagagtggaaggtggaggaaggtgccttgcccagtcttcgtcgcttgcatattgtaaactgcttaaggttgaggacagttccggatggcctgagatttgttgttacactcaaggaaattgtgatcaaacacatgcttaggcaattcaaaaagaaggttgaagaaggaggagaggatttctacaaagtccgtcacgtgacttcacttgtattcaccaatactttggacttctag

SEQ ID NO: 2 - RNA, 2859 nt, Cannabis sativa
  source 1..2859: mol_type = mRNA
atggcagatgctgttgtttcatttgtgattgaaagaattggagacttgctaattgatgaagccaagttcttgtatggagttgaaggccaagtggggaatgcaaaaatcaagcttcaatgtatgagtgctttcttaaaagatgctgatgcttgggtacgtaatggggatgagagagttcgccttttggttgtccaaatcagagaaaattctctagacttggaggatgttgttgagacttatgtcctcaaagtggctttgaaggggaatgaaggaagcaaaagagaaagatttattagcatcttcagagaaggaattgatgtccacaaagttggaacagagattgagaggatctcatccaacattgatacttggacatcgcagttagaagcttttggagtacacagatcaatacacaatgcagctgaagcttcttcaagcagccatgtccaacagcaaagacagttgaggcaagcttattcttatgttgaagacaattatattgttggatttggaaaagatatagaagagttggtaactcttttgactgaaaaagagaattctcgcaagcataaggtgatctctgtatgtgggatgggtggtttgggcaaaactactcttgcaagaaaggtctatcagcatcctcaagtcaggactcactttgagtgttatgcttgggcctcgatatctcagcaatgtaatacacgcaatgtcttcgaagaagtttactttgctttcacttctcccaccaaagaaagaagagaagaaatcaaaaacttaagtgatgttgaattagcaagggagctttacaactttcggaaacagaaaaaatgtttggtagtccttgacgatatatggaccccaacaacatgggatcttctacaacatgcattccctacgactactcaaggagacacattgcatagcaagatcttactcactactcggaataaggttgtagctttgcacgccgatccacatggtttcatccatgaacctcatttgctcaatgacaaggagagttgggagctatttcagaataagtgctattctattggaacagatccatcagattcaaatgatgacgaagaaaggaagaacgaactagcggtagagatgcttaaaaagtgctctggtctgccattagccatcattgtgctcgctggtcttctatctaagaaacacaccttatatgattgggagcaactgaaagcaaatgtaattcgctgcattggtcaaggtgatcaacaacatgatgttgactcaaattaccgtggtgtttcaggggtcttgggtttgagttacagcgagttaccacgtcacttgaagccttgttttctgtacttggctcgttacgcggaagatgtcccaatcagagtaaaagagttatgtcttgtactcatagcagaaggttttatatcgccaagaagagggcctgtggaaactttggaggaagtggcatatgattggttgtgtgagttggtggagagaagtatgattcaggtcaaagaaatgagttcaacagaaggaaggataaagttattttgcattcatgatctgatgagagacttgtgtgtgtctaaagcccaagaagaaaactttctacattttactgattggcagaataaaggagaacagccaatagaagcaaacgtacgaagagtttccatctatgataatagttatagaactattattgatggtagtgattttattcatatgtttagaaacaaagatggctctctcaggtgccttgctctacatggtggaagcactgagaatgaaaaaagaatattgagacatgcatgcaatcactttttgaagcttagagttttgattattggtcatcaacctgtggatatcttttctatttgtttgaagttgcctaaagaaattgggaatctgatccatctaaggttattaagtattcctaattggcgtattaaagagattccatcttcttttggcaatttaagatgcctccagactttgagagtaaggatccagagtgacatcataataccaagttcaatgtgcaagttggagcaactgaggcatctatatcttatcactgtagatggtaatggcatagaatttggtaatttcttgaggtcaactaagtctagaaatttacaaacattggtaggtattcgtaccaaggatcttctactgagtgatcttctacagttggagagtctcaagaaattaggagttgaagtgtatggaaattttgagacattcttacacaatccccaatctctcacattcactcgtcttctgtctttagaaatggataatcgtgttggctttggcaccaagatagatattgttcctttgatattaagctgtcctcacatttataagcttagagtgcgatcacctatagtaagattaccagaagtcaaccaattctccccaaatctcattaagttagagttgagatttctcaaacttgaggatgatccaatgccaacattagaaaagctaccaaaattaagagtccttgtgattgcctatgatagttttagaggggatgagatggtgtgctcaagaggaggtttccctcgacttgaatctcttcagcttactgctctatattacttgaaagagtggaaagtggaggagagtgcattgcctacacttgcctatttgcatattgattgttgcaggaaattgaggagagttccagatgaactaacaaacattgttacactcaatgagatggtcatatcttgtatgcctaagaaattcaaagaaaggatggaggaaggaggagacgatttccacaaagtcaaccacgtgccatcacgtgtattcatcgaatgtgatacagatgaagattcagattcagaagatgactaa

SEQ ID NO: 3 - RNA, 2886 nt, Cannabis sativa
  source 1..2886: mol_type = mRNA
atggcagatgctattgtttcatttgtgattgaaaggcttggagacttggtgatttctgaagctcaattcttgcatggagttgaagcgcaagttgggaatgcacaaatcaagcttcaatgtatgagtgctttcttaaaagatgctgatgcttgcgtaagaaatggtgatgagagagttcgccttttggttgtccaaatcagagaaaattctcttgacttggaggatgttattgaaacttatgtcctcaaggtgactttgaagagaaatgaaggcttactaaagaggtgtatttgcatcttcagaaaaggaattgatgtccacaaagttggatcagagattgagaggatctcatccaacattgatacttggacttcacagttagatgcacttggagtacacagatcgatacacaatgcagctgaagcttcttcaagcagccatgtccaacagcaaagacagttgaggcaagcttattcttatgttgaagacaattatattgttggatttggaaaagatatagaagagttggtaactcttttgactgaaaaagagaattctcgcaagcataaggtgatctctgtatgtgggatgggtggtttgggcaaaactactcttgcaagaaaggtctatcagcatcctcaagtcaggactcactttgagtgttatgcttgggcctcgatatctcagcaatgtaatacacgcaatgtcttcgaagaagtttactttgctttcacttctcccaccaaagaaagaagagaagaaatcaaaaacttaagtgatgttgaattagcaagggagctttacaactttcggaaacagaaaaaatgtttggtagtccttgacgatatatggaccccaacaacatgggatcttctacaacatgcattccctacgactactcaaggagacacattgcatagcaagatcttactcactactcggaataaggttgtagctttgcacgccgatccacatggtttcatccatgaacctcatttgctcaatgacaaggagagttgggagctatttcagaataagtgctattctattggaacagatccatcagattcaaatgatgacgaagaaaggaagaacgaactagcggtagagatgcttaaaaagtgctctggtctgccattagccatcattgtgctcgctggtcttctatctaagaaacacaccttatatgattgggagcaactgaaagcaaatgtaattcgctgcattggtcaaggtgatcaacaacatgatgttgactcaaattaccgtggtgtttcaggggtcttgggtttgagttacagcgagttaccacgtcacttgaagccttgttttctgtacttggctcgttacgcggaagatgtcccaatcagagtaaaagagttatgtcttgtactcatagcagaaggttttatatcgccaagaagagggcctgtggaaactttggaggaagtggcatatgattggttgtgtgagttggtggagagaagtatgattcaggtcaaagaaatgagttcaacagaaggaaggataaagttattttgcattcatgatctgatgagagacttgtgtgtgtctaaagcccaagaagaaaactttctacattttactgattggcagaataaaggagaacagccaatagaagcaaacgtacgaagagtttccatctatgataatagttatagaactattattgatggtagtgattttattcatatgtttagaaacaaagatggctctctcaggtgccttgctctacatggtggaagcactgagaatgaaaaaagaatattgagacatgcatgcaatcactttttgaagcttagagttttgattattggtcatcaacctgtggatatcttttctatttgtttgaagttgcctaaagaaattgggaatctgatccatctaaggttattaagtatttctcattggaaattcgaaacgattccatcttcttttgggaatttaagatgcctgcagagtttgagagtaaggagcaagagtgataaaataccaagttcaatgtgtaagttggagcaactgagacatctatatcttatcactgtagatggtaatggcatagaatttggtaaattcttgaggtcaactaagtctagaaatttacaaacattggtaggcattgacaccgaggatgttctactgagtgatcttctacagttggagagtctcaagaaattaggagttgaagtgtatggaaattttgagacattcttacacaatccccaatctctcacattcactcgtcttttctctttacaagtaaacaatctatttggtgtgaagatagatattattcctttgatattaagctgtcctcacatttataagcttacattgtattcgcctatattaagtttaccacaagtcaaccaattctccccaaatctcattaagttagagttgagatttctcaaacttgaggatgatccaatgccaacattagaaaagctaccaaaattaagagtccttgtgattgcctatgatagttttagaggggatgagatggtgtgctcaagaggaggtttccctcgacttgaatctcttgagcttatttgtctacataacttgaaagagtggaaagtggaggagagtgcattgcctagacttgcctatttgactattaaatggagctgggagttgaggagagttccagatggagtaagaaacattgttacactcaatgagataaagataatttctatgcctaagaaattcaaagaaaggatggaggaaggaggagacgatttccacaaagtcaaccacgtgccatcacgtgtattcatcaattgtgattgggattcaaattcagaagatgctaaactatataagaatttaagtggctgtggctgtggcgtgtggggatag

SEQ ID NO: 4 - RNA, 2892 nt, Cannabis sativa
  source 1..2892: mol_type = mRNA
atggcagagagtctattaatatcccaattagctgaggcagttgtatctcaagcagtaggaagactcagtgagctaataatcaacgaaggtgcatcattgagcagtgtgaaggcagatgtggagaggctacgagatgagctacatcgaatgcactgcttctttgaagatgcagatcgcaggccacaccaccaagacaagcgccttcgccattggatatcagaacttagaggtgttgctaatgatgttgaggatgttattgagatgttcatctccaatgtctcctcttcttgcctccaattcttctacctccgcagactcaggagccaaatcaattcaatcaaaactaagatggaagatatctttaaaagctcccaaacttaccggattgaatctagtagtcgtagtagtagtagtagtagtagaagagttgaagggactagtagttctgtttctgagctacagcggcgtctgaggagatcttgcccagatgacaatgatgatgatgattatgttgtgagcatggagcatagtgtgaaggctttgaaagatgagttgatcatgttgaagaaggaagatgatgatgatcaattgtccatagtttctgtggttggcatgggtggattaggtaagaccactcttgctaagttggtttataaaagcaatgatgttaaacaacactttgattgttgtgcttgggttttcatatgccaacaatatgtttctaaggatattttgtatgaaatcttagttcaaattgataggggtagtgatagagagaaaataaggaatttgaatgagaatgagatgagtgataggcttaagagtgagttaaaagggaagaggtatcttgtggttcttgatgatatttggagaattgaggcttgggattgtatcaaaagagctttccctttaggaaatagtagtagtaaagtgcttttcactactcgaaacaaagaattagcattgtctgttaatccatgtagctctccaattgagccctcttttttaacatttgaagagagctggcaagtacttgtacaaaggcaagcttttcggagaagcggttttggactagaatttgagatgttgggtaaggacatggtgaaaaaatgcggaggactgcctctagcaattgttgttcttggagggctgttgagaacaagaagttcacttgaagaatggaaagaggttcagaaagatgttaactcacacttgaataagctccaatcacatcaagagtatgaaggtgtgaatggcattttagccttgagctactatgatctaccttactacttgaaaccatgttttttgtatttgggaagctttccagaagacagagatatagagaaaaagacattgattcggttatggattgcagagggatttgtacaaaaactgaaaagagaagaggcaaatgaggaaacattggaaaatgtgtgtgaacaatacttgagagagcttacaaataggtgcatgattcaagtgagcaaaaggaacaattcaggagtgggagtgaaaacataccgagtacatgatcttatgcgagacttgtgcgtgtctaaggcgaaagaagagaacttttttcaggtcattcaacatcaaaatgagagtaacatgtcagagcagggaggttcttcaattcatcatggtgctaaaacaatcttttcacggaaaattgttcttcatgttgggtgtgatctcgatttgtgtaaagttcaactccgtctccattcccttttgtgtttcgacaacggtcattttcttgtgagtagcttaagaaagaagactttcatgttcttaagaacactgatacttggaaacacttgtttgggagataacaactttaagattctgaaacaaatctgcacactcatacacttgaggtaccttgggctgcaaaaatctaataatctgcacaaattgccaaaatgcattgccaacttgcgtagcttgcacactttggatcttcgatacagcaatgtcaaaatgctacctacatcaatatcaagactgacacgtctgacacatcttatgagtgataaacctttaatgctcatggacaaagaatcaggtaacaagtttcttaattttgctgttttaagtgttaatcctgctaaaccgacgccaaccaatccggcgcagcctaccacacctatgctcataagggatgtcttaatcttcaaatatgatgttcttctccaacttactaatcttcaagagctaaaaatgctacttagaagcaaagaagaagctacaaaggtgctagaatcggccatcataaaaggcggtagaattcgatccttggatcttgacatggtgtttcatggacatgtacttcaaaatcaactagcaccaccaccaccaatgccttttccaagtctaaaatcactctctaaatgctaccttctcaataaactacgtctaagggggccaatagaaaaagattctttgcaatttctgtccacaagcctagtcaaattgactttggaacatacatgtattcaacaagaccctatggcagtactggaacagctaccaaatttaaagctcctacagttgcattctttttcttatattggttccaaattggtttgctctgccaatggctttcctaaactagagactcttcggctttgtcacttgcaaagactgaaggagtgggaaatagagaaagattccatgcaatgcctgaagagattagagcttgaacaacttctgaaactgaagatgattcctgatggcttgaaattcgttactactcttaggcaattagatgttatagaaatgacacagatttttgaagaaaggattaaagtgagtgaaggacttgaaggggaagattacaacaaagtgcgccacattccctccatctcaactagaagactttattaa

SEQ ID NO: 5 - RNA, 2892 nt, Cannabis sativa; identical to SEQ ID NO: 4
  source 1..2892: mol_type = mRNA

SEQ ID NO: 6 - RNA, 2883 nt, Cannabis sativa
  source 1..2883: mol_type = mRNA
atggcggaaggcatagtcagtttcgtgatcggaagactcggagacttactcataaacgaagctaatttcttgtacggaatccgagaccaagttgaggatgcaaaatctgagctccaacgaatcaaatgctttctcgaagatgccgatgctcgcgccagacacggagacaagtctcttcgtcttcatgtagctgaaatcggtgagtatgcttatgatttggaagatgttatcaccaattttgtattgaaagaatcttctagggttagtaatagtggtagtagcagtagtagtagtagtagaatgttctcttgtggtaatttcatcgaatttcacaaggttggatcgagaattgagaagatttcagctagggtttctaagtcaagattgagttttcaatcttatgggttggttcaatctaggggaaatgatgaagctgctactaatttttcaactaggttagaatttaggcgaactttttctcataatattgattctgattttgttgggtttgaagaaaatattaatgaattggttgagcatttgaagaaaaaagggagaaatttgcatagtgttgtgtcgatttgcggtatgggtggtcttggaaaaactactcttgctagacaggtttactatcatggtgaaattaggcgttattttgattgttttgcttgggcttctgtatctcaaaaatgtcaacctagggatgtttgggaagggattttgattaggttgacatctcctgcatctgaaaggagaagagagattaaagaaatgaaagacgatgaaatcgctaaggctttgttcaaagtttgtactgaaaaaaggtgtcttgttgttcttgatgatatttggagttcttcgacatgggattgcctcaagcttgctttccctaatgttggatcagacagtaaaataatgcttaccactcgtaatagagaggtcgctttccatgcagatcgaaatagcattcttcatgaacccaggtgctttaacaaaaatgaaacttgggagttgtttatgatgaaaactcattttggaacagatgatatcgattcagaggattacaagaggaagaaaaagttatcagaggagatgcttgaatattgtggtggtttgccattagccattacagtgctcggaggacttctatctcgtaaaccaactgcagatgagtgggagacattgcataagaatattaagacatgtataaggaaaggaaaaataaatgagcaagaagactctaactttggtgtctcttgggtgttgggtttaagttacgacgagctaccatactatttaaagccatgttttttgcatttagcttattttccggaagattttgaaataagggctagagatttgtgcctagtatggttggcagaaggcttcgtatcgaaggaggagagagcatatgagtgcttaagtgaattggttgaaaggtgcataattcaagtagcagagtggagctcaactggcacaattaaaacatgtcgtattcatgatctcatgcgggatttatgcttagtaaaggctgaggaggaaggttttttgcagcatgctgatttacgcaatcatcacgaggtaacaaatttttcctcttcctctgttggagctgcaacaaatttagagccaattaataaggttcgtagacttgcgatctacttgaacaacaccagtgttgatgaattggtttctttaataagacataaggatggttgtcttaggtctcttatatgcttcaattcagaaccagatacatcctatgagcaagtgatgaagcccctgttcaatcaattccatttgctgagagttttgaagtttgagaattatactcacggtcatgttggaaagttgcctaaagaaattgggaatcttatccacttaaggctatttagtctcaaggatagttatgtggaaatatttccatcatctataggcaatttgagatgcctgcaaactttagatttgagagttaaaaacctgtatatcgacaaacaacttcaatcttcaaaattaccacctgtcctctggaaattggagcagttgagacatttgtatctgcctcaaaagtatttgacagaagctgagagaagcaagtttacagttactcacaatgagaatattttgcggttgggtaatctaaccaatttgcaaacattggtgaatgtttcggttaattatcattacttgaatgatttggagaaactaaccaatcttaccaaattaaaaataaatttggggtcttgcttccggggcacaagaattaaatttatcaatcttagatctttatccatttgttatgatgacatcatcatcgagagtgaagagtacggtacaataagacgaggagttgaattctcccaccatgagataatacttcttctactgagctgtcctcaacttcataaactgcatctgcaagtttcaatggtgaagttaccagaagaaaatcagttttcttcaaatctcatcaagctaacgctgcttcgaacttgtctcaaggatgatccaatgtcaacattagaaaagctcccgcatttaaggattctcctcctcgatcacaatgccttcctcggacagaaaatgaattgcaccagtaaaggttttcctcgactcgagtctctatccatttgttatctttacgatttcatagagtggaaggtggaggaaggtgccttgcccagtcttcgtcgcttgcatattgtaaactgcttaaggttgaggacagttccagatggcctgagatatgttgttacactcaaagaaattgtgatcaaacacatgcttaggcaattcaagaagaaggttgaagaaggaggagaggatttctacaaagtccatcacgtgccttcacttgtattcaccaatactttggacttctag

SEQ ID NO: 7 - RNA, 2928 nt, Cannabis sativa
  source 1..2928: mol_type = mRNA
aactttcttcttctccaacaacaaaaattccaatctcatccaaaaatggcggaaggcatagtcagtttcgtgatcggaagactcggagacttactcataaacgaagctaatttcttgtacggaatccgagaccaagttgaggatgcaaaatctgagctccaacgaatcaaatgctttctcgaagatgccgatgctcgcgccagacacggagacaagtctcttcgtcttcatgtagctgaaatcggtgagtatgcttatgatttggaagatgttatcaccaattttgtattgaaagaatcttctagggttagtaatagtggtagtagcagtagtagtagtagtagaatgttctcttgtggtaatttcatcgaatttcacaaggttggatcgagaattgagaagatttcagctagggtttctaagtcaagattgagttttcaatcttatgggttggttcaatctaggggaaatgatgaagctgctactaatttttcaactaggttagaatttaggcgaactttttctcataatattgattctgattttgttgggtttgaagaaaatattaatgaattggttgagcatttgaagaaaaaagggagaaatttgcatagtgttgtgtcgatttgcggtatgggtggtcttggaaaaactactcttgctagacaggtttactatcatggtgaaattaggcgttattttgattgttttgcttgggcttctgtatctcaaaaatgtcaacctagggatgtttgggaagggattttgattaggttgacatctcctgcatctgaaaggagaagagagattaaagaaatgaaagacgatgaaatcgctaaggctttgttcaaagtttgtactgaaaaaaggtgtcttgttgttcttgatgatatttggagttcttcgacatgggattgcctcaagcttgctttccctaatgttggatcagacagtaaaataatgcttaccactcgtaatagagaggtcgctttccatgcagatcgaaatagcattcttcatgaacccaggtgctttaacaaaaatgaaacttgggagttgtttatgatgaaaactcattttggaacagatgatatcgattcagaggattacaagaggaagaaaaagttatcagaggagatgcttgaatattgtggtggtttgccattagccattacagtgctcggaggacttctatctcgtaaaccaactgcagatgagtgggagacattgcataagaatattaagacatgtataaggaaaggaaaaataaatgagcaagaagactctaactttggtgtctcttgggtgttgggtttaagttacgacgagctaccatactatttaaagccatgttttttgcatttagcttattttccggaagattttgaaataagggctagagatttgtgcctagtatggttggcagaaggcttcgtatcgaaggaggagagagcatatgagtgcttaagtgaattggttgaaaggtgcataattcaagtagcagagtggagctcaactggcacaattaaaacatgtcgtattcatgatctcatgcgggatttatgcttagtaaaggctgaggaggaaggttttttgcagcatgctgatttacgcaatcatcacgaggtaacaaatttttcctcttcctctgttggagctgcaacaaatttagagccaattaataaggttcgtagacttgcgatctacttgaacaacaccagtgttgatgaattggtttctttaataagacataaggatggttgtcttaggtctcttatatgcttcaattcagaaccagatacatcctatgagcaagtgatgaagcccctgttcaatcaattccatttgctgagagttttgaagtttgagaattatactcacggtcatgttggaaagttgcctaaagaaattgggaatcttatccacttaaggctatttagtctcaaggatagttatgtggaaatatttccatcatctataggcaatttgagatgcctgcaaactttagatttgagagttaaaaacctgtatatcgacaaacaacttcaatcttcaaaattaccacctgtcctctggaaattggagcagttgagacatttgtatctgcctcaaaagtatttgacagaagctgagagaagcaagtttacagttactcacaatgagaatattttgcggttgggtaatctaaccaatttgcaaacattggtgaatgtttcggttaattatcattacttgaatgatttggagaaactaaccaatcttaccaaattaaaaataaatttggggtcttgcttccggggcacaagaattaaatttatcaatcttagatctttatccatttgttatgatgacatcatcatcgagagtgaagagaacggtacaataagacgaggagttaaattctccaactatgagataatacttcttctacggagctgtcctcaacttcataaactgcatctgcaagtttcaatggtgaagttaccagaagaaaatcagttttcttcaaatctcatcaagctaacgctgcttcgaacttgtctcaaggatgatccaatgtcaacattagaaaagctcccgcatttaaggattctcctcctcgatcacaatgccttcctcggacagaaaatgaattgcaccagtaaaggttttcctcgactcgagtctctatccatttcttatctttacgatttcatagagtggaaggtggaggaaggcgccttgcccagtcttcgtcgcttgcatattgtaaactgcttaaggttgaggacagttccagatggcctgagatttgttgttacactcaaggaaattgtgatcaaacacatgcttaggcaattcgaaaagaaggttgaagaaggaggagaggatttctacaaagtccatcacgtgccttcacttgtattcaccaatactttggacttctag

SEQ ID NO: 8 - RNA, 2928 nt, Cannabis sativa; identical to SEQ ID NO: 7
  source 1..2928: mol_type = mRNA

SEQ ID NO: 9 - RNA, 2928 nt, Cannabis sativa; identical to SEQ ID NO: 7
  source 1..2928: mol_type = mRNA

SEQ ID NO: 10 - RNA, 3072 nt, Cannabis sativa
  source 1..3072: mol_type = mRNA
aactttcttcttctccaacaacaaaaattccaatctcatccaaaaatggcggaaggcatagtcagtttcgtgatcggaagactcggagacttactcataaacgaagctaatttcttgtacggaatccgagaccaagttgaggatgcaaaatctgagctccaacgaatcaaatgctttctcgaagatgccgatgctcgcgccagacacggagacaagtctcttcgtcttcatgtagctgaaatcggtgagtatgcttatgatttggaagatgttatcaccaattttgtattgaaagaatcttctagggttagtaatagtggtagtagcagtagtagtagtagtagaatgttctcttgtggtaatttcatcgaatttcacaaggttggatcgagaattgagaagatttcagctagggtttctaagtcaagattgagttttcaatcttatgggttggttcaatctaggggaaatgatgaagctgctactaatttttcaactaggttagaatttaggcgaactttttctcataatattgattctgattttgttgggtttgaagaaaatattaatgaattggttgagcatttgaagaaaaaagggagaaatttgcatagtgttgtgtcgatttgcggtatgggtggtcttggaaaaactactcttgctagacaggtttactatcatggtgaaattaggcgttattttgattgttttgcttgggcttctgtatctcaaaaatgtcaacctagggatgtttgggaagggattttgattaggttgacatctcctgcatctgaaaggagaagagagattaaagaaatgaaagacgatgaaatcgctaaggctttgttcaaagtttgtactgaaaaaaggtgtcttgttgttcttgatgatatttggagttcttcgacatgggattgcctcaagcttgctttccctaatgttggatcagacagtaaaataatgcttaccactcgtaatagagaggtcgctttccatgcagatcgaaatagcattcttcatgaacccaggtgctttaacaaaaatgaaacttgggagttgtttatgatgaaaactcattttggaacagatgatatcgattcagaggattacaagaggaagaaaaagttatcagaggagatgcttgaatattgtggtggtttgccattagccattacagtgctcggaggacttctatctcgtaaaccaactgcagatgagtgggagacattgcataagaatattaagacatgtataaggaaaggaaaaataaatgagcaagaagactctaactttggtgtctcttgggtgttgggtttaagttacgacgagctaccatactatttaaagccatgttttttgcatttagcttattttccggaagattttgaaataagggctagagatttgtgcctagtatggttggcagaaggcttcgtatcgaaggaggagagagcatatgagtgcttaagtgaattggttgaaaggtgcataattcaagtagcagagtggagctcaactggcacaattaaaacatgtcgtattcatgatctcatgcgggatttatgcttagtaaaggctgaggaggaaggttttttgcagcatgctgatttacgcaatcatcacgaggtaacaaatttttcctcttcctctgttggagctgcaacaaatttagagccaattaataaggttcgtagacttgcgatctacttgaacaacaccagtgttgatgaattggtttctttaataagacataaggatggttgtcttaggtctcttatatgcttcaattcagaaccagatacatcctatgagcaagtgatgaagcccctgttcaatcaattccatttgctgagagttttgaagtttgagaattatactcacggtcatgttggaaagttgcctaaagaaattgggaatcttatccacttaaggctatttagtctcaaggatagttatgtggaaatatttccatcatctataggcaatttgagatgcctgcaaactttagatttgagagttaaaaacctgtatatcgacaaacaacttcaatcttcaaaattaccacctgtcctctggaaattggagcagttgagacatttgtatctgcctcaaaagtatttgacagaagctgagagaagcaagtttacagttactcacaatgagaatattttgcggttgggtaatctaaccaatttgcaaacattggtgaatgtttcggttaattatcattacttgaatgatttggagaaactaaccaatcttaccaaattaaaaataaatttggggtcttgcttccggggcacaagaattaaatttatcaatcttagatctttatccatttgttatgatgacatcatcatcgagagtgaagagtacggtacaataagacgaggagttgaattctcccaccatgagataatacttcttctactgagctgtcctcaacttcataaactgcatctgcaagtttcaatggtgaagttaccagaagaaaatcagttttcttcaaatctcatcaagctaacgctgcttcgaacttgtctcaaggatgatccaatgtcaacattagaaaagctcccgcatttaaggattctcctcctcgatcacaatgccttcctcggacagaaaatgaattgcaccagtaaaggttttcctcgactcgagtctctatccatttcttatctttacgatttcatagagtggaaggtggaggaaggcgccttgcccagtcttcgtcgcttgcatattgtaaactgcttaaggttgaggacagttccagatggcctgagatttgttgttacactcaaggaaattgtgatcaaacacatgcttaggcaattcaagaagaaggttgaagaaggtgccttgcccagtcttcgtcgcttgcatattgtaaactgcttaaggttgaggacagttccagatggcctgagatttgttgttacactcaaggaaattgtgatcaaacacatgcttaggcaattcaagaagaaggttgaagaaggaggagaggatttctacaaagtccatcacgtgccttcacttgtattcaccaatactttggacttctag

SEQ ID NO: 11 - RNA, 2817 nt, Cannabis sativa
  source 1..2817: mol_type = mRNA
atggcagatgctgttgtttcgtttgtgattgaaaggcttggcgacttggtgatttctgaagctcaattcttgggtggagttgaggcccaactggggaatgcacagatcaagcttcaatgtatgagtgctttcttaaaagatgctgatgcttgggtaacaaatggtaatgagagagttcgccttttggttgtccagatcagagaaaattcttatgccttggaagatgttattgagacttatgtcctcaaagtgtcttctagtcatggaactatcaaacgtgcattaaaagggtgtgttcgcattattgatgtccacaaagttggatcagagattgagaggatctcatccaacattgatacttggacttcacaattagaagcacttggagtacacagatcaatacaaaaagcagatgaagcttcttcaagcagctatgtccaacagcaaagacagttgaggcaagcttattcttttgttgaagacaatgttgttgttggattcgataaggacattgaagagttggttgcccttttgactgaagaagagaattctcataagcataaggtggtttcgatatgcgggatgggtggtttggggaagactactcttgcaagaaaggtttatcagcattctcaagtcaggactcactttgattgttatgcttgggcctcaatatctcagcaatgtaatacaagcaatgtcttcaaaggaattcacttttctctcacttctcctacagatgaacaaaaaattcacataaagaactcaagtgatgttgaattagcaagggagcttttcaactttctgaaacagaaaaaatgtttggtggttcttgatgatatatggaccacaacaacatgggatcttctaaagcatgcattccctactcaaggagaaacgcatagcaagatcttactcactactcggaacaaggatgtagctttgcatgccgatcaacacggtttcatccatgaacctcgtttgctcaatgaagaggaaagctgggagctgtttcataaaaagtacttctgtgttggagcagatccatcaaactcaaatgatgatgaagaaaggaaaaaagaacttgcagtagagatgcttagaaagtgctctggtctgccattagccatcattgtgctcgctgggcttttatctacgaaaaagaaacacaccgtatatgattgggagcaactgaaagcaaatgtaattcgctggataggtgaaggtggtcaacaacatgatgacaactcaaaatacattagtgttcgggcggtgttaggtttaagttacagtgagttgccatctcatttgaagccttgttttttgtacttggctcgttatgctgaagatgtcccgataagagtaaaagacttatgtcatatacttatagcagaaggtttaatatcacgaagaagaggttatgtggaaacttgggaggaagtggcacatgattggttgtgtgagttggtagagaggagtatgattcaggtgaaagaaatgagttcaagaggaggaaggataaaatcattttgcattcatgatctcatgcgagacttgtgtgtgtctaaagcccaagaagaaaactttctacattttattgattggcagaataaaggggaagagccaatacaaacaaaggtacgaagagttgccatctatgataatggaaagactgttagtagtgattttattcatatggttggaaatatagatgcctctctcaggtgctttgctgtagatggtggaggaaatgagtataatgaaaaaagagtattgagacatgcatgcaatcactttttgaaacttagagttttgattattggttataattccacttatactttgaagttgcctaaagaaattgagaatatgatccatttaaggttattaagtattcctaattggccgattgaaaagattccatcttcttttgggaatttaagatgcctgcagagtttgagagtaaggagcaagagtgataaaataccaagttcaatgtgtaagttggagcaactgagacatctatatcttatcactgtagatggtaatggcatagaatttggtaaattcttgaggtcaactaagtctagaaatttacaaacattggtaagtattcgtaccaacgatcttgtagggagtgatcttctacagttaaagagtctcaagaaattagaaatttgtgtgaatggaaattttgagacattcttacacattccccaatctttcacattctctcgtctttgttctttaaaagtagagagttcaaatcgttacaccgccatagatgtagttcctttgatattaagctgtcctcatatttataagcttagattgtactcggctatattaaggttaccacaagtcaacctattctccccaaatctcataaagttacagttgaaatttctcagacttaaggatgatccaatgccaacattagaaaagctaccaaaattaagagtccttgtgattggctatgatagctttatgggggatgagatggtgtgctcaagaggaggtttccctcgacttgaatctcttgagcttagtgttctacataacttgaaagagtggaaagtggaggagagtgcattggctacacttgccgatttgcgtattcatggatgcaggggattgaggagtgttccagatggaataagaaacattgttacactcaatgagataaagataaataatatgcctaagaaattcaaagaaaggatggaagaaggaggagacgatttccacaaagtcatgcacgtgccatcacgtgtattcatcaattgtgttagagattggccggatgactataaatga

SEQ ID NO: 12 - RNA, 2886 nt, Cannabis sativa
  source 1..2886: mol_type = mRNA
atgttcttagatcatatcatatcatacaattcaattcaattcagttcacttactgagaaaaatcatcgaatggcagatgctgttgtttcatttgtgattgaaaggcttggagacttggtgctttctgaagctcaattcttgggtggagttgaagcccaagtggggaatgcacagatcaagcttcaatgtatgagtgctttcttaaaagatgctgatgcttgggtaacaaatggtaatgagagagttcgccttttggttgtccagatcagagaaaattcttatgccttggaagatgttattgagacttatgtcctcaaagtgtcttctagtcatggaactatcaaacgtgcattaaaagggtgtgttcgcattattgatgtccacaaagttggatcagagattgagaggatctcatccaacattgatacttggacttcacaattagaagcacttggagtacacagatcaatacaaaaagcagatgaagcttcttcaagcagctatgtccaacagcaaagacagttgaggcaagcttattcttttgttgaagacaatgttgttgttggattcgataaggacattgaagagttggttgcccttttgactgaagaagagaattctcataagcataaggtggtttcgatatgcgggatgggtggtttggggaagactactcttgcaagaaaggtttatcagcattctcaagtcaggactcactttgattgttatgcttgggcctcaatatctcagcaatgtaatacaagcaatgtcttcaaaggaattcacttttctctcacttctcctacagatgaacaaaaaattcacataaagaactcaagtgatgttgaattagcaagggagcttttcaactttctgaaacagaaaaaatgtttggtggttcttgatgatatatggaccacaacaacatgggatcttctaaagcatgcattccctactcaaggagaaacgcatagcaagatcttactcactactcggaacaaggatgtagctttgcatgccgatcaacacggtttcatccatgaacctcgtttgctcaatgaagaggaaagctgggagctgtttcataaaaagtacttctgtgttggagcagatccatcaaactcaaatgatgatgaagaaaggaaaaaagaacttgcagtagagatgcttagaaagtgctctggtctgccattagccatcattgtgctcgctgggcttttatctacgaaaaagaaacacaccgtatatgattgggagcaactgaaagcaaatgtaattcgctggataggtgaaggtggtcaacaacatgatgacaactcaaaatacattagtgttcgggcggtgttaggtttaagttacagtgagttgccatctcatttgaagccttgttttttgtacttggctcgttatgctgaagatgtcccgataagagtaaaagacttatgtcatatacttatagcagaaggtttaatatcacgaagaagaggttatgtggaaacttgggaggaagtggcacatgattggttgtgtgagttggtagagaggagtatgattcaggtgaaagaaatgagttcaagaggaggaaggataaaatcattttgcattcatgatctcatgcgagatttgtgcgtgtctagagctcaagaagaaaactttctacattttactgattggcagaataaaggggaagagccaatacaaacaaaggtacgaagagttgccatctatgataatggaaagactgttagtagtgattttattcatatggttggaaatatagatgcctctctcaggtgctttgctgtagatggtggaggaaatgagtataatgaaaaaagagtattgagacatgcatgcaatcactttttgaaacttagagttttgattattggttataattccacttatactttgaagttgcctaaagaaattgagaatatgatccatttaaggttattaagtattcctaattggccgattgaaaagattccatcttcttttgggaatttaagatgcctgcagagtttgagagtaaggagcaagagtgataaaataccaagttcaatgtgtaagttggagcaactgagacatctatatcttatcactgtagatggtaatggcatagaatttggtaaattcttgaggtcaactaagtctagaaatttacaaacattggtaagtattcgtaccaacgatcttgtagggagtgatcttctacagttaaagagtctcaagaaattagaaatttgtgtgaatggaaattttgagacattcttacacattccccaatctttcacattctctcgtcttttttctttaaaagtagagagttcaaatcgttacaccgccatagatgtagttcctttgatattaagctgtcctcatatttataagcttagattgtactcggctatattaaggttaccacaagtcaacctattctccccaaatctcataaagttacagttgaaatttctcagacttaaggatgatccaatgccaacattagaaaagctaccaaaattaagagtccttgtgattggctatgatagctttatgggggatgagatggtgtgctcaagaggaggtttccctcgacttgaatctcttgagcttagtgttctacataacttgaaagagtggaaagtggaggagagtgcattggctacacttgccgatttgcgtattcatggatgcaggggattgaggagtgttccagatggaataagaaacattgttacactcaatgagataaagataaataatatgcctaagaaattcaaagaaaggatggaagaaggaggagacgatttccacaaagtcatgcacgtgccatcacgtgtattcatcaattgtgttagagattggccggatgactataaatga

SEQ ID NO: 13 - RNA, 2928 nt, Cannabis sativa
  source 1..2928: mol_type = mRNA
aactttcttcttctgcaacaacaaaaattccaatctcatccaaaaatggcggaaggcatagtcagtttcgtgatcggaagactcggagacttactcataaacgaagctaatttcttgtacggaatccgagaccaagttgaggatgcaaaatctgagctccaacgaatcaaatgctttctcgaagatgccgatgctcgcgccagacacggagacaagtctcttcgtcttcatgtagctgaaatcggtgagtatgcttatgatttggaagatgttatcaccaattttgtattgaaagaatcttctagggttagtattagtggtagtagcagtagtagtagtagtagaatgttctcttgtggtaatttcatcgaatttcacaaggttggatcgagaattgagaagatttcagctagggtttctaagtcaagattgagttttcaatcttatgggttggttcaatctaggggaaatgatgaagctgctactaatttttcaactaggttagaatttaggcgaactttttctcataatattgattctgattttgttgggtttgaagaaaatattaatgaattggttgagcatttgaagaaaaaagggagaaatttgcatagtgttgtgtcgatttgcggtatgggtggtcttggaaaaactactcttgctagacaggtttactatcatggtgaaattaggcgttattttgattgttttgcttgggcttctgtatctcaaaaatgtcaacctagggatgtttgggaagggattttgattaggttgacatctcctgcatctgaaaggagaagagagattaaagaaatgaaagacgatgaaatcgctaaggctttgttcaaagtttgtactgaaaaaaggtgtcttgttgttcttgatgatatttggagttcttcgacatgggattgcctcaagcttgctttccctaatgttggatcagacagtaaaataatgcttaccactcgtaatagagaggtcgctttccatgcagatcgaaatagcattcttcatgaacccaggtgctttaacaaaaatgaaacttgggagttgtttatgatgaaaactcatttcggaacggatgatatcgattcagaggattacaagaggaagaaaaagttagcaggggagatgcttgaatattgtggtggtttgccattagccattacagtgctcggaggacttctatctcgtaaaccaactgcagatgagtgggagacattgcataagaatattaagacatgtataaggaaaggaaaaataaatgagcaagaagactctaactttggtgtctcttgggtgttgggtttaagttacgacgagctaccatactatttaaagccatgttttttgcatttagcttattttccggaagattttgaaataagggctagagatttgtgcctagtatggttggcagaaggcttcgtatcgaaggaggagagagcatatgagtgcttaagtgaattggttgaaaggtgcataattcaagtagcagagtggagctcaactggcacaattaaaacatgtcgtattcatgatctcatgcgggatttatgcttagtaaaggctgaggaggaaggttttttgcagcatgctgatttacgcaatcatcacgaggtaacaaatttttcctcttcctctgttggagctgcaacaaatttagagccaattaataaggttcgtagacttgcgatctacttgaacaacaccagtgttgatgaattggtttctttaataagacataaggatggttgtcttaggtctcttatatgcttcaattcagaaccagatacatcctatgagcaagtgatgaagcccctgttcaatcaattccatttgctgagagttttgaagtttgagaattatactcacggtcatgttggaaagttgcctaaagaaattgggaatcttatccacttaaggctatttagtctcaaggatagttatgtggaaatatttccatcatctataggcaatttgagatgcctgcaaactttagatttgagagttaaaaacctgtatatcgacaaacaacttcaatcttcaaaattaccacctgtcctctggaaattggagcagttgagacatttgtatctgcctcaaaagtatttgacagaagctgagagaagcaagtttacagttactcacaatgagaatattttgcggttgggtaatctaaccaatttgcaaacattggtgaatgtttcggttaattatcattacttgaatgatttggagaaactaaccaatcttaccaaattaaaaataaatttggggtcttgcttccggggcacaagaattaaatttatcaatcttagatctttatccatttgttatgatgacatcatcatcgagagtgaagagtacggtacaataagacgaggagttgaattctcccaccatgagataatacttcttctactgagctgtcctcaacttcataaactgcatctgcaagtttcaatggtgaagttaccagaagaaaatcagttttcttcaaatctcatcaagctaacgctgcttcgaacttgtctcaaggatgatccaatggcaacattagaaaagctcccgcatttaaggattctcctcctcgatcacaatgccttcctcggacagaaaatgaattgcaccagtaaaggttttcctcgactcgagtctctatccatttgttatctttacgatttcatagagtggaaggtggaggaaggtgccttgcccagtcttcgtcgcttgcatattgtaaactgcttaaggttgaggacagttccagatggcctgagatttgttgttacactcaaggaaattgtgatcaaacacatgcttaggcaattcaagaagaaggttgaagaaggaggagaggatttctacaaagtccatcacgtgccttcacttgtattcaccaatactttggacttctag

SEQ ID NO: 14 - RNA, 2928 nt, Cannabis sativa; identical to SEQ ID NO: 13
  source 1..2928: mol_type = mRNA

SEQ ID NO: 15 - RNA, 2811 nt, Cannabis sativa
  source 1..2811: mol_type = mRNA
atggcagatgctgtagtttcgtttgtgattgaaaggcttggcgacttggtgatttctgaagctcaattcttgggtggagttgaagcccaagtggggaatgcacagatcaagcttcaatgtatgagtgctttcttaaaagatgctgatgcttgggtaacaaatggtaatgagagagttcgccttttggttgtccagatcagagaaaattcttatgccttggaagatgttattgagacttatgtcctcaaagtgtcttctagtcatggaactatcaaacgtgcattaaaagggtgtgttcgcattattgatgtccacaaagttggatcggagattgagaggatctcatccaacattgatacttggacttcacaattagaagcacttggagtacacagatcaatacaaaaagcagatgaagcttcttcaagcagctatgtccaacagcaaagaaagttgaggcaagcttattcttttgttgaagacaatgttgttgttggattcgataaggacattgaagagttggttgcccttttgactgaagaagagaattctcataagcataaggtggtttcgatatgcgggatgggtggtttggggaagactactcttgcaagaaaggtttatcagcattctcaagtcaggactcactttgattgttatgcttgggcctcaatatctcagcaatgtaatacaagcaatgtcttcaaaggaattcacttttctctcacttctcctacagatgaacaaaaaattcacataaaaaactcaagtgatgttgaattagcaagggagcttttcaactttcagaaacagaaaaaatgtttggtggttcttgatgatatatggaccacaacaacatgggatcttctaaagcatgcattccctactcaaggagaaacgcatagcaagatcttactcactactcggaacaaggatgtagctttgcatgccgatcaacacggtttcatccatgaacctcgtttgctcaatgaagaggaaagctgggagctgtttcataaaaagtacttctgtgttgaagcagatccatcaaactcaaatgatgatgaagaaaggaaaaaagaacttgcagtagagatgcttagaaagtgctctggtctgccattagccatcattgtactcgctggtcttctatcaaagaaacacaccatatatgagtgggagctggtgaaaagaaatgtacctcgctgcataggtcaagctgaacaacaacatgatgttgactcaaaccaccatggtgttttcggggtgttaggtttgagttacaacgagttaccaagtaaattgaagccttgttttttgtacttggctcgttatgctgaagatgtcccgataagagtaaaagacttatgtcatatacttatagcagaaggtttaatatcgcgaagaagaggttatgtggaaacttgggaggaagtggcacatgattggttgtgtgagttggtagagaggagtatgattcaggtgaaagaaatgagttcaagaggaggaaggataaaatcattttgcattcatgatctcatgcgagacttgtgtgtgtctaaagcccaagaagaaaactttctacattttactgattggcagaataaatgggaagagccaataaaaacaaaggtacgaagagttgccatctatgataatggaaagactgttagtagtgattttattcatatggttggaaatatagatgcctctctcaggtgctttgctgtagatggtggaggaaatgagtataatgaaaaaagagtattgagacatgcatgcaatcactttttgaaacttagagttttgattattggttataattccatttatactttgaagttgcctaaagaaattgagaatatgatccatttaagattattaagtattcctaattggccgattgaaaagattccatcttcacttgggaatttaagatgcctgcagagtttgacagtaaggagcaagagtgataaaataccaagttcaatgtgtaagttggagcaactgagacatctatatcttatcactgtagatggtaatggcatagaatttggtaaattcttgaggtcaactaagtctagaaatttacaaacattggtaagtattcgtaccaacgatcttgtagggagtgatcttctacagttaaagagtctcaagaaattagaaatttgtgtgaatggaaattttgagacattcttacacattccccaatctttcacattctctcgtctttgttctttaaaagtagagagttcaaatcgttacaccgccatagatgtagttcctttgatattaagctgtcctcatatttataagcttagattgtactcggctatattaaggttaccacaagtcaacctattctccccaaatctcataaagttacagttgaaatttctcagacttaaggatgatccaatgccaacattagaaaagctaccaaaattaagagtccttgtgattggctatgatagctttatgggggatgagatggtgtgctcaagaggaggtttccctcgacttgaatctcttgagcttagtgttctacataacttgaaagagtggaaagtggaggagagtgcattggctacacttgccgatttgcgtattcatggatgcaggggattgaggagtgttccagatggaataagaaacattgttacactcaatgagataaagataaataatatgcctaagaaattcaaagaaaggatggaagaaggaggagacgatttccacaaagtcatgcacgtgccatcacgtgtattcatcaattgtgttagagattggccggatgactataaatga

SEQ ID NO: 16 - RNA, 2817 nt, Cannabis sativa
  source 1..2817: mol_type = mRNA
atggcagatgctgtagtttcgtttgtgattgaaaggcttggcgacttggtgatttctgaagctcaattcttgggtggagttgaggcccaactggggaatgcacagatcaagcttcaatgtatgagtgctttcttaaaagatgctgatgcttgggtaacaaatggtaatgagagagttcgccttttggttgtccagatcagagaaaattcttatgccttggaagatgttattgagacttatgtcctcaaagtgtcttctagtcatggaactatcaaacgtgcattaaaagggtgtgttcgcattattgatgtccacaaagttggatcagagattgagaggatctcatccaacattgatacttggacttcacaattagaagcacttggagtacacagatcaatacaaaaagcagatgaagcttcttcaagcagctatgtccaacagcaaagaaagttgaggcaagcttattcttttgttgaagacaatgttgttgttggattcgataaggacattgaagagttggttgcccttttgactgaagaagagaattctcataagcataaggtggtttcgatatgcgggatgggtggtttggggaagactactcttgcaagaaaggtttatcagcattctcaagtcaggactcactttgattgttatgcttgggcctcaatatctcagcaatgtaatacaagcaatgtcttcaaaggaattcacttttctctcacttctcctacagatgaacaaaaaattcacataaaaaactcaagtgatgttgaattagcaagggagcttttcaactttcagaaacagaaaaaatgtttggtggttcttgatgatatatggaccacaacaacatgggatcttctaaagcatgcattccctactcaaggagaaacgcatagcaagatcttactcactactcggaacaaggatgtagctttgcatgccgatcaacacggtttcatccatgaacctcgtttgctcaatgaagaggaaagctgggagctgtttcataaaaagtacttctgtgttgaagcagatccatcaaactcaaatgatgatgaagaaaggaaaaaagaacttgcagtagagatgcttagaaagtgctctggtctgccattagccatcattgtgctcgctgggcttttatctacgaaaaagaaacacaccgtatatgattgggagcaactgaaagcaaatgtaattcgctggataggtgaaggtggtcaacaacatgatgacaactcaaaatacattagtgttcgggcggtgttaggtttaagttacagtgagttgccatctcatttgaagccttgttttttgtacttggctcgttatgctgaagatgtcccgataagagtaaaagacttatgtcatatacttatagcagaaggtttaatatcgcgaagaagaggttatgtggaaacttgggaggaagtggcacatgattggttgtgtgagttggtagagaggagtatgattcaggtgaaagaaatgagttcaagaggaggaaggataaaatcattttgcattcatgatctcatgagagacttgtgtgtgtctaaagcccaagaagaaaactttctacattttactgattggcagaataaatgggaagagccaataaaaacaaaggtacgaagagttgccatctatgataatggaaagactgttagtagtgattttattcatatggttggaaatatagatgcctctctcaggtgctttgctgtagatggtggaggaaatgagtataatgaaaaaagagtattgagacatgcatgcaatcactttttgaaacttagagttttgattattggttataattccatttatactttgaagttgcctaaagaaattgagaatatgatccatttaagattattaagtattcctaattggccgattgaaaagattccatcttcacttgggaatttaagatgcctgcagagtttgagagtaaggagcaagagtgataaaataccaagttcaatgtgtaagttggagcaactgagacatctatatcttatcactgtagatggtaatggcatagaatttggtaaattcttgaggtcaactaagtctagaaatttacaaacattggtaagtattcgtaccaacgatcttgtagggagtgatcttctacagttaaagagtctcaagaaattagaaatttgtgtgaatggaaattttgagacattcttacacattccccaatctttcacattctctcgtcttttttctttaaaagtagagagttcaaatcgttacaccgccatagatgtagttcctttgatattaagctgtcctcatatttataagcttagattgtactcggctatattaaggttaccacaagtcaacctattctccccaaatctcataaagttacagttgaaatttctcagacttaaggatgatccaatgccaacattagaaaagctaccaaaattaagagtccttgtgattggctatgatagctttatgggggatgagatggtgtgctcaagaggaggtttccctcgacttgaatctcttgagcttagtgttctacataacttgaaagagtggaaagtggaggagagtgcattggctacacttgccgatttgcgtattcatggatgcaggggattgaggagtgttccagatggaataagaaacattgttacactcaatgagataaagataaataatatgcctaagaaattcaaagaaaggatggaagaaggaggagacgatttccacaaagtcatgcacgtgccatcacgtgtattcatcaattgtgttagagattggccggatgactataaatga

SEQ ID NO: 17 - RNA, 2892 nt, Cannabis sativa
  source 1..2892: mol_type = mRNA
atggcagagagtctattaatatcccaattagctgaggcagttgtatctcaagcagtaggaagactcagtgagctaataatcaacgaaggtgcatcattgagcagtgtgaaggcagatgtggagaggctacgagatgagctacatcgaatgcactgcttctttgaagatgcagatcgcaggccacaccaccaagacaagcgccttcgccattggatatcagaacttagaggtgttgctaatgatgttgaggatgttattgagatgttcatctccaatgtctcctcttcttgcctccaattcttctacctccgcagactcaggagccaaatcaattcaatcaaaactaagatggaagatatctttaaaagctcccaaacttaccggattgaatctagtagtcgtagtagtagtagtagtagtagaagagttgaagggactagtagttctgtttctgagctacagcggcgtctgaggagatcttgcccagatgacaatgatgatgatgattatgttgtgagcatggagcatagtgtgaaggctttgaaagatgagttgatcatgttgaagaaggaagatgatgatgatcaattgtccatagtttctgtggttggcatgggtggattaggtaagaccactcttgctaagttggtttataaaagcaatgatgttaaacaacactttgattgttgtgcttgggttttcatatgccaacaatatgtttctaaggatattttgtatgaaatcttagttcaaattgataggggtagtgatagagagaaaataaggaatttgaatgagaatgagatgagtgataggcttaagagtgagttaaaagggaagaggtatcttgtggttcttgatgatatttggagaattgaggcttgggattgtatcaaaagagctttccctttaggaaatagtagtagtaaagtgcttttcactactcgaaacaaagaattagcattgtctgttaatccatgtagctctccaattgagccctcttttttaacatttgaagagagctggcaagtacttgtacaaaggcaagcttttcggagaagcggttttggactagaatttgagatgttgggtaaggacatggtgaaaaaatgcggaggactgcctctagcaattgttgttcttggagggctgttgagaacaagaagttcacttgaagaatggaaagaggttcagaaagatgttaactcacacttgaataagctccaatcacatcaagagtatgaaggtgtgaatggcattttagccttgagctactatgatctaccttactacttgaaaccatgttttttgtatttgggaagctttccagaagacagagatatagagaaaaagacattgattcggttatggattgcagagggatttgtacaaaaactgaaaagagaagaggcaaatgaggaaacattggaaaatgtgtgtgaacaatacttgagagagcttacaaataggtgcatgattcaagtgagcaaaaggaacaattcaggagtgggagtgaaaacataccgagtacatgatcttatgcgagacttgtgcgtgtctaaggcgaaagaagagaacttttttcaggtcattcaacatcaaaatgagagtaacatgtcagagcagggaggttcttcaattcatcatggtgctaaaacaatcttttcacggaaaattgttcttcatgttgggtgtgatctcgatttgtgtaaagttcaactccgtctccattcccttttgtgtttcgacaacggtcattttcttgtgagtagcttaagaaagaagactttcatgttcttaagaacactgatacttggaaacacttgtttgggagataacaactttaagattctgaaacaaatctgcacactcatacacttaaggtaccttgggctgcaaaaatccaataatttgcacaaattgccaaaatgcattgccaacttgcgtagcttgcacactttggatcttcgatacagcaatgtcaaaatgctacctacatcaatatcaagactgacacgtctgacacatcttatgagtgataaacctttaatgctcatggacaaagaatcaggtaacaagtttcttaattttgctgttttaagtgttaatcctgctaaaccgacgccaaccaatccggcgcagcctaccacacctatgctcataagggatgtcttaatcttcaaatatgatgttcttctccaacttactaatcttcaagagctaaaaatgctacttagaagcaaagaagaagctacaaaggtgctagaatcggccatcataaaaggcggtagaattcgatccttggatcttgacatggtgtttcatggacatgtacttcaaaatcaactagcaccaccaccaccaatgccttttccaagtctaaaatcactctctaaatgctaccttctcaataaactacgtctaagggggccaatagaaaaagattctttgcaatttctgtccacaagcctagtcaaattgactttggaacatacatgtattcaacaagaccctatggcagtactggaacagctaccaaatttaaagctcctacagttgcattctttttcttatattggttccaaattggtttgctctgccaatggctttcctaaactagagactcttcggctttgtcacttgcaaagactgaaggagtgggaaatagagaaagattccatgcaatgcctgaagagattagagcttgaacaacttctgaaactgaagatgattcctgatggcttgaaattcgttactactcttaggcaattagatgttatagaaatgacacagatttttgaagaaaggattaaagtgagtgaaggacttgaaggggaagattacaacaaagtgcgccacattccctccatctcaactagaagactttattaa

SEQ ID NO: 18 - RNA, 2883 nt, Cannabis sativa
  source 1..2883: mol_type = mRNA
atggcggaaggcatagtcagtttcgtgatcggaagactcggagacttactcataaacgaagctaatttcttgtacggaatccgagaccaagttgaggatgcaaaatctgagctccaacgaatcaaatgctttctcgaagatgccgatgctcgcgccagacacggagacaagtctcttcgtcttcatgtagctgaaatcggtgagtatgcttatgatttggaagatgttatcaccaattttgtattgaaagaatcttctagggttagtattagtggtagtagcagtagtagtagtagtagaatgttctcttgtggtaatttcatcgaatttcacaaggttggatcgagaattgagaagatttcagctagggtttctaagtcaagattgagttttcaatcttatgggttggttcaatctaggggaaatgatgaagctgctactaatttttcaactaggttagaatttaggcgaactttttctcataatattgattctgattttgttgggtttgaagaaaatattaatgaattggttgagcatttgaagaaaaaagggagaaatttgcatagtgttgtgtcgatttgcggtatgggtggtcttggaaaaactactcttgctagacaggtttactatcatggtgaaattaggcgttattttgattgttttgcttgggcttctgtatctcaaaaatgtcaacctagggatgtttgggaagggattttgattaggttgacatctcctgcatctgaaaggagaagagagattaaagaaatgaaagacgatgaaatcgctaaggctttgttcaaagtttgtactgaaaaaaggtgtcttgttgttcttgatgatatttggagttcttcgacatgggattgcctcaagcttgctttccctaatgttggatcagacagtaaaataatgcttaccactcgtaatagagaggtcgctttccatgcagatcgaaatagcattcttcatgaacccaggtgctttaacaaaaatgaaacttgggagttgtttatgatgaaaactcatttcggaacggatgatatcgattcagaggattacaagaggaagaaaaagttatcagaggagatgcttgaatattgtggtggtttgccattagccattacagtgctcggaggacttctatctcgtaaaccaactgcagatgagtgggagacattgcataagaatattaagacatgtataaggaaaggaaaaataaatgagcaagaagactctaactttggtgtctcttgggtgttgggtttaagttacgacgagctaccatactatttaaagccatgttttttgcatttagcttattttccggaagattttgaaataagggctagagatttgtgcctagtatggttggcagaaggcttcgtatcgaaggaggagagagcatatgagtgcttaagtgaattggttgaaaggtgcataattcaagtagcagagtggagctcaactggcacaattaaaacatgtcgtattcatgatctcatgcgggatttatgcttagtaaaggctgaggaggaaggttttttgcagcatgctgatttacgcaatcatcacgaggtaacaaatttttcctcttcctctgttggagctgcaacaaatttagagccaattaataaggttcgtagacttgcgatctacttgaacaacaccagtgttgatgaattggtttctttaataagacataaggatggttgtcttaggtctcttatatgcttcaattcagaaccagatacatcctatgagcaagtgatgaagcccctgttcaatcaattccatttgctgagagttttgaagtttgagaattatactcacggtcatgttggaaagttgcctaaagaaattgggaatcttatccacttaaggctatttagtctcaaggatagttatgtggaaatatttccatcatctataggcaatttgagatgcctgcaaactttagatttgagagttaaaaacctgtatatcgacaaacaacttcaatcttcaaaattaccacctgtcctctggaaattggagcagttgagacatttgtatctgcctcaaaagtatttgacagaagctgagagaagcaagtttacagttactcacaatgagaatattttgcggttgggtaatctaaccaatttgcaaacattggtgaatgtttcggttaattatcattacttgaatgatttggagaaactaaccaatcttaccaaattaaaaataaatttggggtcttgcttccggggcacaagaattaaatttatcaatcttagatctttatccatttgttatgatgacatcatcatcgagagtgaagagtacggtacaataagacgaggagttgaattctcccaccatgagataatacttcttctactgagctgtcctcaacttcataaactgcatctgcaagtttcaatggtgaagttaccagaagaaaatcagttttcttcaaatctcatcaagctaacgctgcttcgaacttgtctcaaggatgatccaatggcaacattagaaaagctcccgcatttaaggattctcctcctcgatcacaatgccttcctcggacagaaaatgaattgcaccagtaaaggttttcctcgactcgagtctctatccatttgttatctttacgatttcatagagtggaaggtggaggaaggtgccttgcccagtcttcgtcgcttgcatattgtaaactgcttaaggttgaggacagttccagatggcctgagatttgttgttacactcaaggaaattgtgatcaaacacatgcttaggcaattcaagaagaaggttgaagaaggaggagaggatttctacaaagtccatcacgtgccttcacttgtattcaccaatactttggacttctag

SEQ ID NO: 19 - RNA, 2883 nt, Cannabis sativa
  source 1..2883: mol_type = mRNA
atggcggaaggcatagtcagtttcgtgatcggaagactcggagacttactcataaacgaagctaatttcttgtacggaatccgagaccaagttgaggatgcaaaatctgagctccaacgaatcaaatgctttctcgaagatgccgatgctcgcgccagacacggagacaagtctcttcgtcttcatgtagctgaaatcggtgagtatgcttatgatttggaagatgttatcaccaattttgtattgaaagaatcttctagggttagtattagtggtagtagcagtagtagtagtagtagaatgttctcttgtggtaatttcatcgaatttcacaaggttggatcgagaattgagaagatttcagctagggtttctaagtcaagattgagttttcaatcttatgggttggttcaatctaggggaaatgatgaagctgctactaatttttcaactaggttagaatttaggcgaactttttctcataatattgattctgattttgttgggtttgaagaaaatattaatgaattggttgagcatttgaagaaaaaagggagaaatttgcatagtgttgtgtcgatttgcggtatgggtggtcttggaaaaactactcttgctagacaggtttactatcatggtgaaattaggcgttattttgattgttttgcttgggcttctgtatctcaaaaatgtcaacctagggatgtttgggaagggattttgattaggttgacatctcctgcatctgaaaggagaagagagattaaagaaatgaaagacgatgaaatcgctaaggctttgttcaaagtttgtactgaaaaaaggtgtcttgttgttcttgatgatatttggagttcttcgacatgggattgcctcaagcttgctttccctaatgttggatcagacagtaaaataatgcttaccactcgtaatagagaggtcgctttccatgcagatcgaaatagcattcttcatgaacccaggtgctttaacaaaaatgaaacttgggagttgtttatgatgaaaactcatttcggaacggatgatatcgattcagaggattacaagaggaagaaaaagttagcaggggagatgcttgaatattgtggtggtttgccattagccattacagtgctcggaggacttctatctcgtaaaccaactgcagatgagtgggagacattgcataagaatattaagacatgtataaggaaaggaaaaataaatgagcaagaagactctaactttggtgtctcttgggtgttgggtttaagttacgacgagctaccatactatttaaagccatgttttttgcatttagcttattttccggaagattttgaaataagggctagagatttgtgcctagtatggttggcagaaggcttcgtatcgaaggaggagagagcatatgagtgcttaagtgaattggttgaaaggtgcataattcaagtagcagagtggagctcaactggcacaattaaaacatgtcgtattcatgatctcatgcgggatttatgcttagtaaaggctgaggaggaaggttttttgcagcatgctgatttacgcaatcatcacgaggtaacaaatttttcctcttcctctgttggagctgcaacaaatttagagccaattaataaggttcgtagacttgcgatctacttgaacaacaccagtgttgatgaattggtttctttaataagacataaggatggttgtcttaggtctcttatatgcttcaattcagaaccagatacatcctatgagcaagtgatgaagcccctgttcaatcaattccatttgctgagagttttgaagtttgagaattatactcacggtcatgttggaaagttgcctaaagaaattgggaatcttatccacttaaggctatttagtctcaaggatagttatgtggaaatatttccatcatctataggcaatttgagatgcctgcaaactttagatttgagagttaaaaacctgtatatcgacaaacaacttcaatcttcaaaattaccacctgtcctctggaaattggagcagttgagacatttgtatctgcctcaaaagtatttgacagaagctgagagaagcaagtttacagttactcacaatgagaatattttgcggttgggtaatctaaccaatttgcaaacattggtgaatgtttcggttaattatcattacttgaatgatttggagaaactaaccaatcttaccaaattaaaaataaatttggggtcttgcttccggggcacaagaattaaatttatcaatcttagatctttatccatttgttatgatgacatcatcatcgagagtgaagagtacggtacaataagacgaggagttgaattctcccaccatgagataatacttcttctactgagctgtcctcaacttcataaactgcatctgcaagtttcaatggtgaagttaccagaagaaaatcagttttcttcaaatctcatcaagctaacgctgcttcgaacttgtctcaaggatgatccaatggcaacattagaaaagctcccgcatttaaggattctcctcctcgatcacaatgccttcctcggacagaaaatgaattgcaccagtaaaggttttcctcgactcgagtctctatccatttgttatctttacgatttcatagagtggaaggtggaggaaggtgccttgcccagtcttcgtcgcttgcatattgtaaactgcttaaggttgaggacagttccagatggcctgagatttgttgttacactcaaggaaattgtgatcaaacacatgcttaggcaattcaagaagaaggttgaagaaggaggagaggatttctacaaagtccatcacgtgccttcacttgtattcaccaatactttggacttctag

SEQ ID NO: 20 - RNA, 2868 nt, Cannabis sativa
  source 1..2868: mol_type = mRNA
atggcagatgctgttgtttcatttgtgattgaaagaattggagacttgctaattgatgaagccaagttcttgtatggagttgaaggccaagtggggaatgcaaaaatcaagcttcaatgtatgagtgctttcttaaaagatgctgatgcttgggtaacaaatggtaatgagagagttcgccttttggttgtccaaatcagagaaaattctctagacttggaggatgttgttgagacttatgtcctcaaagtggctttgaaggggaatgaaggaagcaaaagagaaagatttattagcatcttcagagaaggaattgatgtccacaaagttggatcggagattgagaggatctcatccaacattgatacttggacatcgcagttagaagcttttggagtacacagatcaatacacaatgcagctgaagcttcttcaagcagccatgtccaacagcaaagacagttgaggcaagcttattcttatgttgaagacaattatattgttggatttggaaaagatatagaagagttggtaactcttttgactgaaaaagagaattctcgcaagcataaggtgatctctgtatgtgggatgggtggtttgggcaaaactactcttgcaagaaaggtctatcagcatcctcaagtcaggactcactttgagtgttatgcttgggcctcgatatctcagcaatgtaatacacgcaatgtcttcgaagaagtttactttgctttcacttctcccaccaaagaaagaagagaagaaatcaaaaacttaagtgatgttgaattagcaagggagctttacaactttcggaaacagaaaaaatgtttggtagtccttgacgatatatggaccccaacaacatgggatcttctacaacatgcattccctactactactcaaggagacgcacatagcaagatcttactcactactcggaataaggttgtagctttgcacgccgatccacatggtttcatccatgaacctcatttgctcaatgacaaggagagttgggagctatttcagaataagtgctattctattggaacagatccatcagattcaaatgatgacgaagaaaggaagaacgaactagcggtagagatgcttaaaaagtgctctggtctgccattagccatcattgtgctcgctggtcttctatctaagaaacacaccttatatgattgggagcaactgaaagcaaatgtaattcgctggataggtgaaggtggtcaacaacatgatgacaactcaaaatacattagtgttcgggcggtgttaggtttaagttacagtgagttgccatctcatttgaagccttgttttttgtacttggctcgttatgctgaagatgtcccgataagagtaaaagacttatgtcatatacttatagcagaaggtttaatatcgcgaagaagaggttatgtggaaacttgggaggaagtggcacatgattggttgtgtgagttggtagagaggagtatgattcaggtgaaagaaatgagttcaagaggaggaaggataaaatcattttgcattcatgatctcatgcgagacttgtgtgtgtctaaagcccaagaagaaaactttctacattttactgattggcagaataaatgggaagagccaataaaaacaaaggtacgaagagttgccatctatgataatggaaagactgttagtagtgattttattcatatggttggaaatatagatgcctctctcaggtgctttgctgtagatggtggaggaaatgagtataatgaaaaaagagtattgagacatgcatgcaatcactttttgaaacttagagttttgattattggttataattccatttatactttgaagttgcctaaagaaattgagaatatgatccatttaagattattaagtattcctaattggccgattgaaaagattccatcttcacttgggaatttaagatgcctgcagagtttgacagtaaggagcaagagtgataaaataccaagttcaatgtacaagttggagcaactgaggcatctatatcttatcactgtagatggtaagggcatagaatttggtaaattcttgaggtcaactaagtctagaaatttacaaacattggtaagtattcgtaccaacgatcttgtagggagtgatcttctacagttaaagagtctcaagaaattagaaatttgtgtgaatggaaattttgagacattcttacacaatccccaatctctcacattcactcgtcttttctctttacaagtaaacaatctatttggtgtgaagatagatattattcctttgatattaagctgtcctcacatttataagcttacattgtattcgcctatattaagtttaccacaagtcaaccaattctccccaaatctcatcaagttaacgttgagatttctcaaacttgaggatgatccaatgccaacattagaaaagctaccaaaattaagagtccttatgattggatatgatagcttcaaaggggatgagatggtgtgctcaagaggaggtttccctcgacttgaatctcttgagcttagtgttctacataacttgaaagagtggaaagtggaggagagtgcattggctacacttgccgatttgcgtattcatggatgcaggggattgaggagtgttccagatggaataagaaacattgttacactcaatgagataaagataatttctatgcctaagaaattcaaagaaaggatggaggaaggaggagacgatttccacaaagtcaaccacgtgccatcacgtgtattcatcaattgtgattgggattcaaattcagaagatgctaaactatataagaatttaagtggctgtggctgtggcgtgtggggatag

SEQ ID NO: 21 - RNA, 2877 nt, Cannabis sativa
  source 1..2877: mol_type = mRNA
atggcagatgctgttgtttcatttgtgattgaaagaattggagacttgctaattgatgaagccaagttcttgtatggagttgaaggccaagtggggaatgcaaaaatcaagcttcaatgtatgagtgctttcttaaaagatgctgatgcttgggtacgtaatggggatgagagagttcgccttttggttgtccaaatcagagaaaattctctagacttggaggatgttgttgagacttatgtcctcaaagtggctttgaaggggaatgaaggaagcaaaagagaaagatttattagcatcttcagagaaggaattgatgtccacaaagttggaacagagattgagaggatctcatccaacattgatacttggacatcgcagttagaagcttttggagtacacagatcaatacacaatgcagctgaagcttcttcaagcagccatgtccaacagcaaagacagttgaggcaagcttattcttatgttgaagacaattatattgttggatttggaaaagatatagaagagttggtaactcttttgactgaaaaagagaattctcgcaagcataaggtgatctctgtatgtgggatgggtggtttgggcaaaactactcttgcaagaaaggtctatcagcatcctcaagtcaggactcactttgagtgttatgcttgggcctcgatatctcagcaatgtaatacacgcaatgtcttcgaagaagtttactttgctttcacttctcccaccaaagaaagaagagaagaaatcaaaaacttaagtgatgttgaattagcaagggagctttacaactttcggaaacagaaaaaatgtttggtagtccttgacgatatatggaccccaacaacatgggatcttctacaacatgcattccctactactactcaaggagacgcacatagcaagatcttactcactactcggaataaggttgtagctttgcacgccgatccacatggtttcatccatgaacctcatttgctcaatgacaaggagagttgggagctatttcagaataagtgctattctattggaacagatccatcagattcaaatgatgacgaagaaaggaagaacgaactagcggtagagatgcttaaaaagtgctctggtctgccattagccatcattgtgctcgctggtcttctatctaagaaacacaccttatatgattgggagcaactgaaagcaaatgtaattcgctgcattggtcaaggtgatcaacaacatgatgttgactcaaattaccgtggtgtttcaggggtcttgggtttgagttacagcgagttaccacgtcacttgaagccttgttttctgtacttggctcgttacgcggaagatgtcccaatcagagtaaaagagttatgtcttgtactcatagcagaaggttttatatcgttaagaagaggttctgtggaaactttggaggaagtggcatatgattggttgtgtgagttggtggagagaagtatgattcaggtcaaagaaatgagttcaacagaaggaaggataaagttattttgcattcatgatctgatgagagacttgtgtgtgtctaaagcccaagaagaaaactttctacattttactgattggcagaataaaggagaacagccaatagaagcaaacgtacgaagagtttccatctatgataatagttatagaactattattgatggtagtgattttattcatatgtttagaaacaaagatggctctctcaggtgccttgctctacatggtggaagcactgagaatgaaaaaagaatattgagacatgcatgcaatcactttttgaagcttagagttttgattattggtcatcaacctgtggatatcttttctatttgtttgaagttgcctaaagaaattgggaatctgatccatctaaggttattaagtattccttattggccaattagaaagattccatcttcttttggcaatttaagatgcctccagactttgagagtatcgaccttgagtcataacataccaagttcaatgtgcaagttggagcaactgaggcatctacatttttctgatcggggcataaaatttggtaaattcttgaggtcaactaagtctagaaatttacaaacattggtaggcattgacaccgaggatgttctactgagtgatcttctacagttggagagtctcaagaaattaggagttgaagtgtatggaaattttgagacattcttacacaatccccaatctctcacattcactcgtcttttctctttacaagtaaacaatctatttggtgtgaagatagatattattcctttgatattaagctgtcctcacatttataagcttacattgtattcgcctatattaagtttaccacaagtcaaccaattctccccaaatctcatcaagttaacgttgagatttctcaaacttgaggatgatccaatgccaacattagaaaagctaccaaaattaagagtccttatgattggatatgatagcttcaaaggggatgagatggtgtgctcaagaggaggtttccctcgacttgaatctcttcagcttattggtctagaaaacttgaaagagtggaaagtggaggagagtgcattgcctagacttgcctatttgactattaaatggagctgggagttgaggagagttccagatggagtaagaaacattgttacactcaatgagataaagataatttctatgcctaagaaattcaaagaaaggatggaggaaggaggagacgatttccacaaagtcaaccacgtgccatcacgtgtattcatcaattgtgattgggattcaaattcagaagatgctaaactatataagaatttaagtggctgtggctgtggcgtgtggggatag

SEQ ID NO: 22 - RNA, 2886 nt, Cannabis sativa
  source 1..2886: mol_type = mRNA
atggcagagagtctattaatatcccaattagctgaggcagttgtatctcaagcagtaggaagactcagtgagctaataatcaacgaaggtgcatcattgagcagtgtgaaggcagatgtggagaggctacgagatgagctacatcgaatgcactgcttctttgaagatgcagatcgcaggccacaccaccaagacaagcgccttcgccattggatatcagaacttagaggtgttgctaatgatgttgaggatgttattgagatgttcatctccaatgtctcctcttcttgcctccaattcttctacctccgcagactcaggagccaaatcaattccatcaaaactaagatggaagatatctttaaaagctcccaaacttaccggattgaatctagtagtcgtagtagtagtagtagaagagttgaagggactagtagttctgtttctgagctacagcggcgtctgaggagatcttgcccagatgacaatgatgatgatgattatgttgtgagcatggagcatagtgtgaaggctttgaaagatgagttgatcatgttgaagaaggaagatgatgatgatcaattgtccatagtttctgtggttggcatgggtggattaggtaagaccactcttgctaagttggtttataaaagcaatgatgttaaacaacactttgattgttgtgcttgggttttcatatgccaacaatatgtttctaaggatattttgtatgaaatcttagttcaaattgataggggtagtgatagagagaaaataaggaatttgaatgagaatgagatgagtgataggcttaagagtgagttaaaagggaagaggtatcttgtggttcttgatgatatttggagaattgaggcttgggattgtatcaaaagagcttttcctttaggaaatagtagtagtaaagtgcttttcactactcgaaacaaagaattagcattgtctgttaatccatgtagctctccaattgagccctcttttttaacatttgaagagagctggcaagtacttgtacaaaggcaagcttttcggagaagcggttttggactagaatttgagatgttgggtaaggacatggtgaaaaaatgcggaggactgcctctagcaattgttgttcttggagggctgttgagaacaagaagttcacttgaagaatggaaagaggttcagaaagatgttaactcacacttgaataagctccaatcacatcaagagtatgaaggtgtgaatggcattttagccttgagctactatgatctaccttactacttgaaaccatgttttttgtatttgggaagctttccagaagacagagatatagagaaaaagacattgattcggttatggattgcagagggatttgtacaaaaaccgaaaagagaagaggcaaatgaggaaacattggaaaatgtgtgtgaacaatacttgagagagcttacaaataggtgcatgattcaagtgagcaaaaggaacaattcaggagtgggagtgaaaacataccgagtacatgatcttatgcgagacttgtgcgtgtctaaggcgaaagaagagaacttttttcaggtcattcaacatcaaaatgagagtaacatgtcagagcagggaggttcttcaattcatcatggtgctaaaacaatcttttcacggaaaattgttcttcatgttgggtgtgatctcgatttgtgtaaagttcaactccgtctccattcccttttgtgtttcgacaacggtcattttcttgtgagtagcttaagaaagaagactttcatgttcttaagaacactaatacttggaaacacttgtttgggagataacaactttaagattctgaaacaaatctgcacactcatacacttgaggtaccttgggctgcaaaaatctaataatctgcacaaattgccaaaatgcattgccaacttgcgtagcttgcacactttggatcttcgatacagcaatgtcaaaatgctacctacatcaatatcaagactgacacgtctgacacatcttatgagtgataaacctttaatgctcatggacaaagaatcaggtaacaagtttcttaattttgctgttttaagtgttaatcctgctaaaccgacgccaaccaatccggcgcagcctaccacacctatgctcataagggatgtcttaatcttcaaatatgatgttcttctccaacttactaatcttcaagagctaaaaatgctacttagaagcaaagaagaagctacaaaggtgctagaatcggccatcataaaaggcggtagaattcgatccttggatcttgacatggtgtttcatggacatgtacttcaaaatcaactagcaccaccaccaccaatgccttttccaagtctaaaatcactctctaaatgctaccttctcaataaactacgtctaagggggccaatagaaaaagattctctgcaatttctgtccacaagcttagtcaaattgactttggaacatacatgtattcaacaagaccctatggcagtactggaacagctaccaaatttaaagctcctacagttgcattctttttcttatattggttccaaattggtttgctctgccaatggctttcctaaactagagactcttcggctttgtcacttgcaaagactgaaggagtgggaaatagagaaagattccatgcaatgcctgaagagattagagcttgaacaacttctgaaactgaagatgattcctgatggcttgaaattcgttactactcttaggcaattagatgttatagaaatgacacagatttttgaagaaaggattaaagtgagtgaaggacttgaaggggaagattacaacaaagtgcgccacattccctccatctcaactagaagactttattaa

SEQ ID NO: 23 - RNA, 2820 nt, Cannabis sativa
  source 1..2820: mol_type = mRNA
atggcagatgctgttgtttcgtttgtgattgaaaggcttggagacttggtgatttctgaagctcaattcttgggtggagttgaagcccaagtggggaatgcacagattaagcttcaatgtatgagtgctttcttaaaagatgctgatgcttgggttagaaatggtgatgagagagttcgccttttggttgtccaagtcagagaaaatgcttatgccttggaggatgttattgagacttatgtcctcaaagtgactttgaagaagaatgaaggtgtactcaaaagatctattaacatcttcagagagggaattgatgtccacaaagttggatcagagattgagaggatctcatccaacattgatacttggacttcacaattagaagcacttggagtacacagatcaatacaaaaagcagatgaagcttcttcaagcagctatgtccaacagcaaagaaagttgaggcaagcttattcttttgttgaagacaatgttgttgttggattcgacaaagatattaaagatttggttggccttttgactgaaaaagagaaccctcataagcataaggtgatctctgtatgtgggatgggtggtttgggcaaaactactcttgcaaaaaaggtctatcagcatcctcaagtcaggactcactttgattgttatgcttgggcctcaatatctcagcaatgtaatagacgcgatgtcttcgaagaagtttactttgctttcacttctcccacagatgcacaaagaaaacgcatcaaaaacttaagtgatgttgaattagcaagggagctttacaactttcagaaacagaaaaaatgtttggtagttcttgatgatatatggactgctacaacatgggatcttctacaacatgcattccctactactactcaaggagacacattgcatagcaagatcttactcactactcggaacaaggatgtagctttgcatgccgatcaacacggtttcatccatgaacctcacttgctcaatgaagaggaaagctgggagctgtttcataaaaagtacttctcagttggagcagatccatcaaactcaaattatgatgaagaaaggaagaaagaacttgcggtagagatgcttagaaagtgctctggtctgccattagccatcattgtgctcgctggtcttctatctaagaaacacaccttatatgattgggagcaactgaaagcaaatgtaattcgctggataggtcaaggtgctcaacaacattatgatgactcaaaataccgtagtgttcggggggtgttgggtttgagttacagcgagttaccacgtcacttgaagccttgttttctgtacttggctcgttacgctgaagatgtcccaataagagcaaaagagttatatcttgtgctcatagcagaaggttttatatcgcgaagaggagggtctgtggaaactttggaggaagtggcatatgattggttgtgtgagttggtggagaggagtatgattcaggtcaaagaaatgagtttaacagaaggaaggataaaatcattttgcattcatgatctcatgcgagacttgtgtgtgtctaaagcccaagaagaaaactttctacattttactgattggcagaataaaggagaacagccaatagaagcaaacgtacgaagagtttccatctatgataatagttatagtattattgatggtagtgattttattcatatgtttagaaacaaagatggctctctcaggtgccttgctctatattgtgaaagaattgagaaaagaaaaagaatattgagacatgcatgcaatcactttttgaagcttagagttttgattattggtgaccggtatcttttcaagactttgaagttgcctaaagaaattgggaatctgatccatctaaggttattaagtattcctaattgggaaataaaaaatattccatcttcttttggcaatttaagatgtctccagactttgagagtatggaccaggagttataaaataccaagttcaatgtggaagttggagcaattgaggcatctatatttcactccagttggcatagaatttggtaatttcttgaggtcaactaagtctagaaatttacaaacattggtaggtattcgtaccaaggatcttctactgagtgatctgctacagttggagagtctcaagaaattagggattcgtgtggatcgaaattttgacagattcctacacaagccccaaactctcacattcactcgtcttcagtctttacaaatgaataatcgttttggcaccaagatagatattgttcctttgatattaagctgtcctcacatttataagcttagagtagagttgcctataataagattaccagaagtcaaccaattctccccaaatctcatcaagttaaggttgtttgatctcagacttgaggatgatccaatgccaacattagaaaagctaccaaaattaagagtccttgtgattggctatgatagctttatgggggatgagatggtgtgctcaagaggaggtttccctcgacttgaatctcttgagcttagtgttctacataacttgaaagagtggaaagtggaggagagtgcattgcctacacttgcctatttgcgtatttattgttgtaggaaattgaggagagttccagatggagtaagaaacattgttacactcaatgagatggtcatagattatatgcctaagaaattcaaagaaaggatggaggaaggaggagacgatttccacaaagtcaaccacgtgccatcacgtgtattcatggaatgtgatacagattggcttccagaagatgactaa

SEQ ID NO: 24 - RNA, 2820 nt, Cannabis sativa
  source 1..2820: mol_type = mRNA
atggcagatgctgttgtttcgtttgtgattgaaaggcttggagacttggtgatttctgaagctcaattcttgggtggagttgaagcccaagtggggaatgcacagattaagcttcaatgtatgagtgctttcttaaaagatgctgatgcttgggttagaaatggtgatgagagagttcgccttttggttgtccaagtcagagaaaatgcttatgccttggaggatgttattgagacttatgtcctcaaagtgactttgaagaagaatgaaggtgtactcaaaagatctattaacatcttcagagagggaattgatgtccacaaagttggatcagagattgagaggatctcatccaacattgatacttggacttcacaattagaagcacttggagtacacagatcaatacaaaaagcagatgaagcttcttcaagcagctatgtccaacagcaaagaaagttgaggcaagcttattcttttgttgaagacaatgttgttgttggattcgacaaagatattaaagatttggttggccttttgactgaaaaagagaaccctcataagcataaggtgatctctgtatgtgggatgggtggtttgggcaaaactactcttgcaaaaaaggtctatcagcatcctcaagtcaggactcactttgattgttatgcttgggcctcaatatctcagcaatgtaatagacgcgatgtcttcgaagaagtttactttgctttcacttctcccacagatgcacaaagaaaacgcatcaaaaacttaagtgatgttgaattagcaagggagctttacaactttcagaaacagaaaaaatgtttggtagttcttgatgatatatggactgctacaacatgggatcttctacaacatgcattccctactactactcaaggagacacattgcatagcaagatcttactcactactcggaacaaggatgtagctttgcatgccgatcaacacggtttcatccatgaacctcacttgctcaatgaagaggaaagctgggagctgtttcataaaaagtacttctcagttggagcagatccatcaaactcaaattatgatgaagaaaggaagaaagaacttgcggtagagatgcttagaaagtgctctggtctgccattagccatcattgtgctcgctggtcttctatctaagaaacacaccttatatgattgggagcaactgaaagcaaatgtaattcgctggataggtcaaggtgctcaacaacattatgatgactcaaaataccgtagtgttcggggggtgttgggtttgagttacagcgagttaccacgtcacttgaagccttgttttctgtacttggctcgttacgctgaagatgtcccaataagagcaaaagagttatatcttgtgctcatagcagaaggttttatatcgcgaagaggagggtctgtggaaactttggaggaagtggcatatgattggttgtgtgagttggtggagaggagtatgattcaggtcaaagaaatgagtttaacagaaggaaggataaaatcattttgcattcatgatctcatgcgagacttgtgtgtgtctaaagcccaagaagaaaactttctacattttactgattggcagaataaaggagaacagccaatagaagcaaacgtacgaagagtttccatctatgataatagttatagtattattgatggtagtgattttattcatatgtttagaaacaaagatggctctctcaggtgccttgctctatattgtgaaagaattgagaaaagaaaaagaatattgagacatgcatgcaatcactttttgaagcttagagttttgattattggtgaccggtatcttttcaagactttgaagttgcctaaagaaattgggaatctgatccatctaaggttattaagtattcctaattgggaaataaaaaatattccatcttcttttggcaatttaagatgtctccagactttgagagtatggaccaggagttataaaataccaagttcaatgtggaagttggagcaattgaggcatctatatttcactccagttggcatagaatttggtaatttcttgaggtcaactaagtctagaaatttacaaacattggtaggtattcgtaccaaggatcttctactgagtgatctgctacagttggagagtctcaagaaattagggattcgtgtggatcgaaattttgacagattcctacacaagccccaaactctcacattcactcgtcttcagtctttacaaatgaataatcgttttggcaccaagatagatattgttcctttgatattaagctgtcctcacatttataagcttagagtagagttgcctataataagattaccagaagtcaaccaattctccccaaatctcatcaagttaaggttgtttgatctcagacttgaggatgatccaatgccaacattagaaaagctaccaaaattaagagtccttgtgattggctatgatagctttatgggggatgagatggtgtgctcaagaggaggtttccctcgacttgaatctctgaggcttattagtctagaaaacttgaaagagtggaaagtggaggagagtgcattgcctacacttgcctatttgcgtatttattgttgtaggaaattgaggagagttccagatggagtaagaaacattgttacactcaatgagatggtcatagattatatgcctaagaaattcaaagaaaggatggaggaaggaggagacgatttccacaaagtcaaccacgtgccatcacgtgtattcatggaatgtgatacagattggcttccagaagatgactaa

SEQ ID NO: 25 - RNA, 2820 nt, Cannabis sativa
  source 1..2820: mol_type = mRNA
atggcagatgctgttgtttcgtttgtgattgaaaggcttggagacttggtgatttctgaagctcaattcttgggtggagttgaagcccaagtggggaatgcacagattaagcttcaatgtatgagtgctttcttaaaagatgctgatgcttgggttagaaatggtgatgagagagttcgccttttggttgtccaaatcagagaaaaatctcttgacttggaggatgttattgaaacttatgttctcaaggtgactttgaggagaaatgaaggcttactaaagaggtgtatttgcatcttcagaaaaggaattgatgtccacaaagttggatcagagattgagaggatctcatccgacattcatacttggacttcacagttagaagcttttggagtacgcagatcaatacacaatgcagctgaagcttcttcaagcagctatgtccaacagcaaagaaagttgaggcaagcttattcttttgttgaagacaatgttgttgttggattcgacaaagatattaaagatttggttggccttttgactgaaaaagagaaccctcataagcataaggtgatctctgtatgtgggatgggtggtttgggcaaaactactcttgcaaaaaaggtctatcagcatcctcaagtcaggactcactttgattgttatgcttgggcctcaatatctcagcaatgtaatagacgcgatgtcttcgaagaagtttactttgctttcacttctcccacagatgcacaaagaaaacgcatcaaaaacttaagtgatgttgaattagcaagggagctttacaactttcagaaacagaaaaaatgtttggtagttcttgatgatatatggactgctacaacatgggatcttctacaacatgcattccctactactactcaaggagacacattgcatagcaagatcttactcactactcggaacaaggatgtagctttgcatgccgatcaacacggtttcatccatgaacctcacttgctcaatgaagaggaaagctgggagctgtttcataaaaagtacttctcagttggagcagatccatcaaactcaaattatgatgaagaaaggaagaaagaacttgcggtagagatgcttagaaagtgctctggtctgccattagccatcattgtgctcgctggtcttctatctaagaaacacaccttatatgattgggagcaactgaaagcaaatgtaattcgctggataggtcaaggtgctcaacaacattatgatgactcaaaataccgtagtgttcggggggtgttgggtttgagttacagcgagttaccacgtcacttgaagccttgttttctgtacttggctcgttacgctgaagatgtcccaataagagcaaaagagttatatcttgtgctcatagcagaaggttttatatcgcgaagaggagggtctgtggaaactttggaggaagtggcatatgattggttgtgtgagttggtggagaggagtatgattcaggtcaaagaaatgagtttaacagaaggaaggataaaatcattttgcattcatgatctcatgcgagacttgtgtgtgtctaaagcccaagaagaaaactttctacattttactgattggcagaataaaggagaacagccaatagaagcaaacgtacgaagagtttccatctatgataatagttatagtattattgatggtagtgattttattcatatgtttagaaacaaagatggctctctcaggtgccttgctctatattgtgaaagaattgagaaaagaaaaagaatattgagacatgcatgcaatcactttttgaagcttagagttttgattattggtgaccggtatcttttcaagactttgaagttgcctaaagaaattgggaatctgatccatctaaggttattaagtattcctaattgggaaataaaaaatattccatcttcttttggcaatttaagatgtctccagactttgagagtatggaccaggagttataaaataccaagttcaatgtggaagttggagcaattgaggcatctatatttcactccagttggcatagaatttggtaatttcttgaggtcaactaagtctagaaatttacaaacattggtaggtattcgtaccaaggatcttctactgagtgatctgctacagttggagagtctcaagaaattagggattcgtgtggatcgaaattttgacagattcctacacaagccccaaactctcacattcactcgtcttcagtctttacaaatgaataatcgttttggcaccaagatagatattgttcctttgatattaagctgtcctcacatttataagcttagagtagagttgcctataataagattaccagaagtcaaccaattctccccaaatctcatcaagttaaggttgtttgatctcagacttgaggatgatccaatgccaacattagaaaagctaccaaaattaagagtccttgtgattggctatgatagctttatgggggatgagatggtgtgctcaagaggaggtttccctcgacttgaatctctgaggcttattagtctagaaaacttgaaagagtggaaagtggaggagagtgcattgcctacacttgcctatttgcgtatttattgttgtaggaaattgaggagagttccagatggagtaagaaacattgttacactcaatgagatggtcatagattatatgcctaagaaattcaaagaaaggatggaggaaggaggagacgatttccacaaagtcaaccacgtgccatcacgtgtattcatggaatgtgatacagattggcttccagaagatgactaa

SEQ ID NO: 26 - RNA, 2844 nt, Cannabis sativa
  source 1..2844: mol_type = mRNA
atggcagatgctgttgtttcgtttgtgattgaaaggcttggagacttggtgatttctgaagctcaattcttgggtggagttgaagcccaagtggggaatgcacagattaagcttcaatgtatgagtgctttcttaaaagatgctgatgcttgggttagaaatggtgatgagagagttcgccttttggttgtccaaatcagagaaaaatctcttgacttggaggatgttattgaaacttatgttctcaaggtgactttgaggagaaatgaaggcttactaaagaggtgtatttgcatcttcagaaaaggaattgatgtccacaaagttggatcagagattgagaggatctcatccgacattcatacttggacttcacagttagaagcttttggagtacgcagatcaatacacaatgcagctgaagcttcttcaagcagctatgtccaacagcaaagaaagttgaggcaagcttattcttttgttgaagacaatgttgttgttggattcgacaaagatattaaagatttggttggccttttgactgaaaaagagaaccctcataagcataaggtgatctctgtatgtgggatgggtggtttgggcaaaactactcttgcaaaaaaggtctatcagcatcctcaagtcaggactcactttgattgttatgcttgggcctcaatatctcagcaatgtaatagacgcgatgtcttcgaagaagtttactttgctttcacttctcccacagatgcacaaagaaaacgcatcaaaaacttaagtgatgttgaattagcaagggagctttacaactttcagaaacagaaaaaatgtttggtagttcttgatgatatatggactgctacaacatgggatcttctacaacatgcattccctactactactcaaggagacacattgcatagcaagatcttactcactactcggaacaaggatgtagctttgcatgccgatcaacacggtttcatccatgaacctcacttgctcaatgaagaggaaagctgggagctgtttcataaaaagtacttctcagttggagcagatccatcaaactcaaattatgatgaagaaaggaagaaagaacttgcggtagagatgcttagaaagtgctctggtctgccattagccatcattgtgctcgctggtcttctatctaagaaacacaccttatatgattgggagcaactgaaagcaaatgtaattcgctggataggtcaaggtgctcaacaacattatgatgactcaaaataccgtagtgttcggggggtgttgggtttgagttacagcgagttaccacgtcacttgaagccttgttttctgtacttggctcgttacgctgaagatgtcccaataagagcaaaagagttatatcttgtgctcatagcagaaggttttatatcgcgaagaggagggtctgtggaaactttggaggaagtggcatatgattggttgtgtgagttggtggagaggagtatgattcaggtcaaagaaatgagtttaacagaaggaaggataaaatcattttgcattcatgatctcatgcgagacttgtgtgtgtctaaagcccaagaagaaaactttctacattttactgattggcagaataaaggagaacagccaatagaagcaaacgtacgaagagtttccatctatgataatagttatagtattattgatggtagtgattttattcatatgtttagaaacaaagatggctctctcaggtgccttgctctatattgtgaaagaattgagaaaagaaaaagaatattgagacatgcatgcaatcactttttgaagcttagagttttgattattggtgaccggtatcttttcaagactttgaagttgcctaaagaaattgggaatctgatccatctaaggttattaagtattcctaattgggaaataaaaaatattccatcttcttttggcaatttaagatgtctccagactttgagagtatggaccaggagttataaaataccaagttcaatgtggaagttggagcaattgaggcatctatatttcactccagttggcatagaatttggtaatttcttgaggtcaactaagtctagaaatttacaaacattggtaggtattcgtaccaaggatcttctactgagtgatctgctacagttggagagtctcaagaaattagggattcgtgtggatcgaaattttgacagattcctacacaagccccaaactctcacattcactcgtcttcagtctttacaaatgaataatcgttttggcaccaagatagatattgttcctttgatattaagctgtcctcacatttataagcttagagtagagttgcctataataagattaccagaagtcaaccaattctccccaaatctcatcaagttaaggttgtttgatctcagacttgaggatgatccaatgccaacattagaaaagctaccaaaattaagagtccttgtgattggctatgatagctttatgggggatgagatggtgtgctcaagaggaggtttccctcgacttgaatctctgaggcttattagtctagaaaacttgaaagagtggaaagtggaggagagtgcattgcctacacttgcctatttgcgtatttattgttgtaggaaattgaggagagttccagatggagtaagaaacattgttacactcaatgagatggtcatagattatatgcctaagaaattcaaagaaaggatggaggaaggaggagacgatttcgatcaagtcaagcgcgtgccatcacgtgtatttatcaactgtgatcaagaatcagaggatagtgtcaatgctatatacaatcaactcaacctttag

SEQ ID NO: 27 - RNA, 2814 nt, Cannabis sativa
  source 1..2814: mol_type = mRNA
atggcagatgctgttgtttcgtttgtgattgaaaggcttggagacttggtgatttctgaagctcaattcttgggtggagttgaagcccaagtggggaatgcacagattaagcttcaatgtatgagtgctttcttaaaagatgctgatgcttgggtaacaaatggtgatgagagagttcgccttttggttgtccaaatcagagaaaaatctcttgacttggaggatgttattgaaacttatgttctcaaggtgactttgaggagaaatgaaggcttactaaagaggtgtatttgcatcttcagaaaaggaattgatgtccacaaagttggatcagagattgagaggatctcatccgacattcatacttggacttcacagttagaagcttttggagtacgcagatcaatacacaatgcagctgaagcttcttcaagcagctatgtccaacagcaaagaaagttgaggcaagcttattcttttgttgaagacaatgttgttgttggattcgacaaagatattaaagatttggttggccttttgactgaaaaagagaaccctcataagcataaggtgatctctgtatgtgggatgggtggtttgggcaaaactactcttgcaaaaaaggtctatcagcatcctcaagtcaggactcactttgattgttatgcttgggcctcaatatctcagcaatgtaatagacgcgatgtcttcgaagaagtttactttgctttcacttctcccacagatgcacaaagaaaacgcatcaaaaacttaagtgatgttgaattagcaagggagctttacaactttcagaaacagaaaaaatgtttggtagttcttgatgatatatggactgctacaacatgggatcttctacaacatgcattccctactactactcaaggagacacattgcatagcaagatcttactcactactcggaacaaggatgtagctttgcatgccgatcaacacggtttcatccatgaacctcacttgctcaatgaagaggaaagctgggagctgtttcataaaaagtacttctcagttggagcagatccatcaaactcaaattatgatgaagaaaggaagaaagaacttgcggtagagatgcttagaaagtgctctggtctgccattagccatcattgtgctcgctggtcttctatctaagaaacacaccttatatgattgggagcaactgaaagcaaatgtaattcgctggataggtcaaggtgctcaacaacattatgatgactcaaaataccgtagtgttcggggggtgttgggtttgagttacagcgagttaccacgtcacttgaagccttgttttctgtacttggctcgttacgctgaagatgtcccaataagagcaaaagagttatatcttgtgctcatagcagaaggttttatatcgcgaagaggagggtctgtggaaactttggaggaagtggcatatgattggttgtgtgagttggtggagaggagtatgattcaggtcaaagaaatgagtttaacagaaggaaggataaaatcattttgcattcatgatctcatgcgagacttgtgtgtgtctaaagcccaagaagaaaactttctacattttactgattggcagaataaaggagaacagccaatagaagcaaacgtacgaagagtttccatctatgataatggaaatattgatggtagtgattttattcatatgtttagaaacaaagatggctctctcaggtgccttgctctatattgtgaaagaattgagaaaagaaaaagaatattgagacatgcatgcaatcactttttgaagcttagagttttgattattggtgaccggtatcttttcaagactttgaagttgcctaaagaaattgggaatctgatccatctaaggttattaagtattccttattggccaattagaaagattccatcttcttttggcaatttaagatgtctccagactttgagagtatggaccaggagttataaaataccaagttcaatgtggaagttggagcaattgaggcatctatatttcactccagttggcatagaatttggtaatttcttgaggtcaactaagtctagaaatttacaaacattggtaggtattcgtaccaaggatcttctactgagtgatctgctacagttggagagtctcaagaaattagggattcgtgtggatcgaaattttgacagattcctacacaagccccaaactctcacattcactcgtcttcagtctttacaaatgaataatcgttttggcaccaagatagatattgttcctttgatattaagctgtcctcacatttataagcttagagtaaacttgtctatagtaagattaccacaagtcaaccaattctccccaaatctcatcaagttaaggttgtttgatctcagacttgaggatgatccaatgccaacattagaaaagctaccaaaattaagagtccttgagattggtgttgatagctttagaggggatgagatggtgtgctcaagaggaggtttccctcgacttgaatctctgaggcttattagtctagaaaacttgaaagagtggaaagtggaggagagtgcattgcctacacttgcctatttgcgtatttattgttgtaggaaattgaggagagttccagatggagtaagaaacattgttacactcaatgagataaagataatttgtatgcctaagaaattcaaagaaaggatggaggaaggaggagacgatttccacaaagtcaaccacgtgccatcacgtgtattcatggaatgtgatacagattggcttccagaagatgactaa

SEQ ID NO: 28 - RNA, 2706 nt, Cannabis sativa
  source 1..2706: mol_type = mRNA
atggcagaatatgttgttcccctagtggtagaaaagttggcaagccaacttatggaggaagccatttccttgtcaaaagggaaggaacatgtcgaatggatcgaagcagaactaaggcgaatgcagtgctttctcaaagatgcagatgctaagcaggataaggatgcaaggattagcaattgggtggtagatataagagaagttgcttttgatgcagatgatttcattgatacttatatttacaaaacaaatggacaaagagagaagggtcttgtcatgtgtttcaagaggtactctcatctatttttcttatgcgaattagtttctaacttcaaaatcaacaagaatatcagtaggatcaaatcaaaaatccatgaaatagcagatagcaaattaacttatggtattgaaaacataaggcaaaagagtgaaggggcaactttaacagttagtgaactacaggagaggagaatatcttctcctcatcattttgaggatgatattattggtttggatgaagacataaaaatattagaatctcagttgatttatggacagtcgtgtcgatctgtactctccataattggtatggcaggtttaggcaagaccactttggctaagatgatttaccatagcaatggaataaaaaataattttgattgttgtgcttggatttatgtctctcaagaatatagtgcagaagaagttttgagggatttggcgaaaaaagtcatgggcttttctaagatagagttggaaaagatgcatagagaagatttggtagaagaaatttcgaagtttctacagaggagaagatatgtcatagttttggatgacatatggaagaaacaagtgtgggataatctgaaagatgcttttcctgactcgaaaaatggaagtcgaatcatgctcactactcgtttcaaagatattgctatacatgctgatccgagaagtccaccccatgaaccaagtcttcttaatgatgaggaaagttggcaactactttccagaaagatctccctacaatggaatgcagttacaagtttgcctccatggactgatgagcttggaaaaaaaattgttaaaaaatgtgggggattgcctctagcgattgtagtactaggtggactcttgtcgagaagggaagcttcctatagtgagtggctcaaggttttgcaaagtgttcattggcagctaagacaagacactatacaatgcgcagacatcttagctttaagctactttgatttgcctttctacctgaagccatgctttctttactttggtctttttcctgaagactttgagatatctgctagtaggttgatcttgttgtgggttgcagaaggatttgtgcaaccaagaggtcaagagccaatagaagatgtctctgaagactacttagaagagcttgtaggaagaagcatggttcaagttgctacaaggaactcaaacggaaggatcaaaacttgtcgtatgcatgatcttttgagagaactcgctatagcaaaagctacagaagacagatttcttcatgtcatccatggtgaaagtagagctggttctataaggaccctctcaagaagtcgtcgacttgctatacacttcaattttcccccatctaagaagacaacaatgaatttacgctccttgttatgctttgacctcaatgagccagttaaagttcaagtaaagaggtttaaactactgagagttcttgatttagaaggttcatatatatctaagcttgactcagctattggaaagcttattcatttaagatatttgggactgagaggtacttggttgaaaaatctcccacgttctctacgctatctttccaatttggaaactctagatctgagatcaactctaattaatccattcccagatgagattttgaggctgcaacaactgagacacttgtattttagtgatgtccaaccaatggtgtcaataccacccaccatccctagtacatatctccctgacctccagacattaaagggtttatttatcagtgagattagtagaagtgatatagaggatttcttgagcaagctaaccacaatgagatcattgagtttgcatggtcgtctttgtattaagaaagaacacttgggaaattgggttgccaagttgaaaagccttgaatgtctaaagctcaatgcaacaagacttatgaccgaggatacaattcatgcagcagttccggagtgggatttgtcatgcctcactcatctttacaagatacatttagcggggagtatgcaaaagatgtttgacatacaatacttcccaacaaatctttctgagttgaccttgacacaatcctggctaatggaagatcccatgaagacgcttgggaaactgagaagcctgagagttttgaaactaaagcatgcagcatacgtaggcagagaaatcacttgctcaagtggaggctttgttcagcttcaattgttgaagctctcattcatgaactcagtagaatccttgagaatagaggaaggagcaatgccaaatctgagagaattggagattgttcagtgtaaattgaagattgttccaagaggcctatggccagtgcaatctctcaaaagcttgaagttgggatttatgcctggtcatgatattggacttaagattcaagaacgtgagggagagaattggtatagaatagaacatgttcttccaatataa

SEQ ID NO: 29 - protein, 960 aa, Cannabis sativa
MAEAIVSFVIGRLGDLLINESNFLYGIRDQVEDAKSELQRIKCFLEDADARARHGDKSLRLHVAEIGEYAYDLEDVITNFVLKESSRVSNSGSSSSSSSRMFSCGNFIEFHKVGSRIEKISARVSKSRLSFQSYGLVQSRGNDEAATNFSTRLEFRRTFSHNIDSDFVGFEENINELVEHLKKKGRNLHSVVSICGMGGLGKTTLARQVYYHGEIRPYFDCFAWASVSQKCQPRDVWEGILIRLTSPASERRREIKEMKDDEIAKALFKVCTEKRCLVVLDDIWSSSTWDCLKLAFPNVGSDSKIMLTTRNREVAFHADRNSILHEPRCFNKNETWELFMMKTHFGTDDIDSEDYKRKKKLSEEMLEYCGGLPLAITVLGGLLSRKPTADEWETLHKNIKTCIRKGKINEQEDSNFGVSWVLGLSYDELPYYLKPCFLHLAYFPEDFEIRARDLCLVWLAEGFVSKEERAYECLSELVERCIIQVAEWSSTGTIKTCRIHDLMRDLCLVKAEEEGFLQHADLRNHHEVTNFSSSSVGAATNLEPINKVRRLAIYLNNTSVDELVSLIRHKDGCLRSLICFNSEPDTSYEQVMKPPFNQFHLLRVLKFENYTHGHVGKLPKEIGNLIHLRLFSLKDSYVEIFPSSVGNLRCLQILDLRVKNLYIDKQLQSSKLPPVLWKLEQLRHLYLPQKYLTEAERSKFTVTHNENILRLGNLTNLQTLVNVSVNYHYLNDLEKLTNLTKLKINLGSCFRGTRIKFINLRSLSICYDDIIIESEEYGTIRRGVEFSHHEIILLLLSCPQLHKLHLQVSMVKLPEENQFSSNLIKLTLLRTCLKDDPMATLEKLPHLRILLLDHNAFLGQKMNCTSKGFPRLESLSICYLYDFIEWKVEEGALPSLRRLHIVNCLRLRTVPDGLRFVVTLKEIVIKHMLRQFKKKVEEGGEDFYKVRHVTSLVFTNTLDF

SEQ ID NO: 30 - protein, 952 aa, Cannabis sativa
MADAVVSFVIERIGDLLIDEAKFLYGVEGQVGNAKIKLQCMSAFLKDADAWVRNGDERVRLLVVQIRENSLDLEDVVETYVLKVALKGNEGSKRERFISIFREGIDVHKVGTEIERISSNIDTWTSQLEAFGVHRSIHNAAEASSSSHVQQQRQLRQAYSYVEDNYIVGFGKDIEELVTLLTEKENSRKHKVISVCGMGGLGKTTLARKVYQHPQVRTHFECYAWASISQQCNTRNVFEEVYFAFTSPTKERREEIKNLSDVELARELYNFRKQKKCLVVLDDIWTPTTWDLLQHAFPTTTQGDTLHSKILLTTRNKVVALHADPHGFIHEPHLLNDKESWELFQNKCYSIGTDPSDSNDDEERKNELAVEMLKKCSGLPLAIIVLAGLLSKKHTLYDWEQLKANVIRCIGQGDQQHDVDSNYRGVSGVLGLSYSELPRHLKPCFLYLARYAEDVPIRVKELCLVLIAEGFISPRRGPVETLEEVAYDWLCELVERSMIQVKEMSSTEGRIKLFCIHDLMRDLCVSKAQEENFLHFTDWQNKGEQPIEANVRRVSIYDNSYRTIIDGSDFIHMFRNKDGSLRCLALHGGSTENEKRILRHACNHFLKLRVLIIGHQPVDIFSICLKLPKEIGNLIHLRLLSIPNWRIKEIPSSFGNLRCLQTLRVRIQSDIIIPSSMCKLEQLRHLYLITVDGNGIEFGNFLRSTKSRNLQTLVGIRTKDLLLSDLLQLESLKKLGVEVYGNFETFLHNPQSLTFTRLLSLEMDNRVGFGTKIDIVPLILSCPHIYKLRVRSPIVRLPEVNQFSPNLIKLELRFLKLEDDPMPTLEKLPKLRVLVIAYDSFRGDEMVCSRGGFPRLESLQLTALYYLKEWKVEESALPTLAYLHIDCCRKLRRVPDELTNIVTLNEMVISCMPKKFKERMEEGGDDFHKVNHVPSRVFIECDTDEDSDSEDD

SEQ ID NO: 31 - protein, 961 aa, Cannabis sativa
MADAIVSFVIERLGDLVISEAQFLHGVEAQVGNAQIKLQCMSAFLKDADACVRNGDERVRLLVVQIRENSLDLEDVIETYVLKVTLKRNEGLLKRCICIFRKGIDVHKVGSEIERISSNIDTWTSQLDALGVHRSIHNAAEASSSSHVQQQRQLRQAYSYVEDNYIVGFGKDIEELVTLLTEKENSRKHKVISVCGMGGLGKTTLARKVYQHPQVRTHFECYAWASISQQCNTRNVFEEVYFAFTSPTKERREEIKNLSDVELARELYNFRKQKKCLVVLDDIWTPTTWDLLQHAFPTTTQGDTLHSKILLTTRNKVVALHADPHGFIHEPHLLNDKESWELFQNKCYSIGTDPSDSNDDEERKNELAVEMLKKCSGLPLAIIVLAGLLSKKHTLYDWEQLKANVIRCIGQGDQQHDVDSNYRGVSGVLGLSYSELPRHLKPCFLYLARYAEDVPIRVKELCLVLIAEGFISPRRGPVETLEEVAYDWLCELVERSMIQVKEMSSTEGRIKLFCIHDLMRDLCVSKAQEENFLHFTDWQNKGEQPIEANVRRVSIYDNSYRTIIDGSDFIHMFRNKDGSLRCLALHGGSTENEKRILRHACNHFLKLRVLIIGHQPVDIFSICLKLPKEIGNLIHLRLLSISHWKFETIPSSFGNLRCLQSLRVRSKSDKIPSSMCKLEQLRHLYLITVDGNGIEFGKFLRSTKSRNLQTLVGIDTEDVLLSDLLQLESLKKLGVEVYGNFETFLHNPQSLTFTRLFSLQVNNLFGVKIDIIPLILSCPHIYKLTLYSPILSLPQVNQFSPNLIKLELRFLKLEDDPMPTLEKLPKLRVLVIAYDSFRGDEMVCSRGGFPRLESLELICLHNLKEWKVEESALPRLAYLTIKWSWELRRVPDGVRNIVTLNEIKIISMPKKFKERMEEGGDDFHKVNHVPSRVFINCDWDSNSEDAKLYKNLSGCGCGVWG

SEQ ID NO: 32 - protein, 963 aa, Cannabis sativa
MAESLLISQLAEAVVSQAVGRLSELIINEGASLSSVKADVERLRDELHRMHCFFEDADRRPHHQDKRLRHWISELRGVANDVEDVIEMFISNVSSSCLQFFYLRRLRSQINSIKTKMEDIFKSSQTYRIESSSRSSSSSSRRVEGTSSSVSELQRRLRRSCPDDNDDDDYVVSMEHSVKALKDELIMLKKEDDDDQLSIVSVVGMGGLGKTTLAKLVYKSNDVKQHFDCCAWVFICQQYVSKDILYEILVQIDRGSDREKIRNLNENEMSDRLKSELKGKRYLVVLDDIWRIEAWDCIKRAFPLGNSSSKVLFTTRNKELALSVNPCSSPIEPSFLTFEESWQVLVQRQAFRRSGFGLEFEMLGKDMVKKCGGLPLAIVVLGGLLRTRSSLEEWKEVQKDVNSHLNKLQSHQEYEGVNGILALSYYDLPYYLKPCFLYLGSFPEDRDIEKKTLIRLWIAEGFVQKLKREEANEETLENVCEQYLRELTNRCMIQVSKRNNSGVGVKTYRVHDLMRDLCVSKAKEENFFQVIQHQNESNMSEQGGSSIHHGAKTIFSRKIVLHVGCDLDLCKVQLRLHSLLCFDNGHFLVSSLRKKTFMFLRTLILGNTCLGDNNFKILKQICTLIHLRYLGLQKSNNLHKLPKCIANLRSLHTLDLRYSNVKMLPTSISRLTRLTHLMSDKPLMLMDKESGNKFLNFAVLSVNPAKPTPTNPAQPTTPMLIRDVLIFKYDVLLQLTNLQELKMLLRSKEEATKVLESAIIKGGRIRSLDLDMVFHGHVLQNQLAPPPPMPFPSLKSLSKCYLLNKLRLRGPIEKDSLQFLSTSLVKLTLEHTCIQQDPMAVLEQLPNLKLLQLHSFSYIGSKLVCSANGFPKLETLRLCHLQRLKEWEIEKDSMQCLKRLELEQLLKLKMIPDGLKFVTTLRQLDVIEMTQIFEERIKVSEGLEGEDYNKVRHIPSISTRRLY

SEQ ID NO: 33 - protein, 963 aa, Cannabis sativa; identical to SEQ ID NO: 32

SEQ ID NO: 34 - protein, 960 aa, Cannabis sativa
MAEGIVSFVIGRLGDLLINEANFLYGIRDQVEDAKSELQRIKCFLEDADARARHGDKSLRLHVAEIGEYAYDLEDVITNFVLKESSRVSNSGSSSSSSSRMFSCGNFIEFHKVGSRIEKISARVSKSRLSFQSYGLVQSRGNDEAATNFSTRLEFRRTFSHNIDSDFVGFEENINELVEHLKKKGRNLHSVVSICGMGGLGKTTLARQVYYHGEIRRYFDCFAWASVSQKCQPRDVWEGILIRLTSPASERRREIKEMKDDEIAKALFKVCTEKRCLVVLDDIWSSSTWDCLKLAFPNVGSDSKIMLTTRNREVAFHADRNSILHEPRCFNKNETWELFMMKTHFGTDDIDSEDYKRKKKLSEEMLEYCGGLPLAITVLGGLLSRKPTADEWETLHKNIKTCIRKGKINEQEDSNFGVSWVLGLSYDELPYYLKPCFLHLAYFPEDFEIRARDLCLVWLAEGFVSKEERAYECLSELVERCIIQVAEWSSTGTIKTCRIHDLMRDLCLVKAEEEGFLQHADLRNHHEVTNFSSSSVGAATNLEPINKVRRLAIYLNNTSVDELVSLIRHKDGCLRSLICFNSEPDTSYEQVMKPLFNQFHLLRVLKFENYTHGHVGKLPKEIGNLIHLRLFSLKDSYVEIFPSSIGNLRCLQTLDLRVKNLYIDKQLQSSKLPPVLWKLEQLRHLYLPQKYLTEAERSKFTVTHNENILRLGNLTNLQTLVNVSVNYHYLNDLEKLTNLTKLKINLGSCFRGTRIKFINLRSLSICYDDIIIESEEYGTIRRGVEFSHHEIILLLLSCPQLHKLHLQVSMVKLPEENQFSSNLIKLTLLRTCLKDDPMSTLEKLPHLRILLLDHNAFLGQKMNCTSKGFPRLESLSICYLYDFIEWKVEEGALPSLRRLHIVNCLRLRTVPDGLRYVVTLKEIVIKHMLRQFKKKVEEGGEDFYKVHHVPSLVFTNTLDF

SEQ ID NO: 35 - protein, 975 aa, Cannabis sativa
NFLLLQQQKFQSHPKMAEGIVSFVIGRLGDLLINEANFLYGIRDQVEDAKSELQRIKCFLEDADARARHGDKSLRLHVAEIGEYAYDLEDVITNFVLKESSRVSNSGSSSSSSSRMFSCGNFIEFHKVGSRIEKISARVSKSRLSFQSYGLVQSRGNDEAATNFSTRLEFRRTFSHNIDSDFVGFEENINELVEHLKKKGRNLHSVVSICGMGGLGKTTLARQVYYHGEIRRYFDCFAWASVSQKCQPRDVWEGILIRLTSPASERRREIKEMKDDEIAKALFKVCTEKRCLVVLDDIWSSSTWDCLKLAFPNVGSDSKIMLTTRNREVAFHADRNSILHEPRCFNKNETWELFMMKTHFGTDDIDSEDYKRKKKLSEEMLEYCGGLPLAITVLGGLLSRKPTADEWETLHKNIKTCIRKGKINEQEDSNFGVSWVLGLSYDELPYYLKPCFLHLAYFPEDFEIRARDLCLVWLAEGFVSKEERAYECLSELVERCIIQVAEWSSTGTIKTCRIHDLMRDLCLVKAEEEGFLQHADLRNHHEVTNFSSSSVGAATNLEPINKVRRLAIYLNNTSVDELVSLIRHKDGCLRSLICFNSEPDTSYEQVMKPLFNQFHLLRVLKFENYTHGHVGKLPKEIGNLIHLRLFSLKDSYVEIFPSSIGNLRCLQTLDLRVKNLYIDKQLQSSKLPPVLWKLEQLRHLYLPQKYLTEAERSKFTVTHNENILRLGNLTNLQTLVNVSVNYHYLNDLEKLTNLTKLKINLGSCFRGTRIKFINLRSLSICYDDIIIESEENGTIRRGVKFSNYEIILLLRSCPQLHKLHLQVSMVKLPEENQFSSNLIKLTLLRTCLKDDPMSTLEKLPHLRILLLDHNAFLGQKMNCTSKGFPRLESLSISYLYDFIEWKVEEGALPSLRRLHIVNCLRLRTVPDGLRFVVTLKEIVIKHMLRQFEKKVEEGGEDFYKVHHVPSLVFTNTLDF

SEQ ID NO: 36 - protein, 975 aa, Cannabis sativa; identical to SEQ ID NO: 35

SEQ ID NO: 37 - protein, 975 aa, Cannabis sativa; identical to SEQ ID NO: 35

SEQ ID NO: 38 - protein, 1023 aa, Cannabis sativa
NFLLLQQQKFQSHPKMAEGIVSFVIGRLGDLLINEANFLYGIRDQVEDAKSELQRIKCFLEDADARARHGDKSLRLHVAEIGEYAYDLEDVITNFVLKESSRVSNSGSSSSSSSRMFSCGNFIEFHKVGSRIEKISARVSKSRLSFQSYGLVQSRGNDEAATNFSTRLEFRRTFSHNIDSDFVGFEENINELVEHLKKKGRNLHSVVSICGMGGLGKTTLARQVYYHGEIRRYFDCFAWASVSQKCQPRDVWEGILIRLTSPASERRREIKEMKDDEIAKALFKVCTEKRCLVVLDDIWSSSTWDCLKLAFPNVGSDSKIMLTTRNREVAFHADRNSILHEPRCFNKNETWELFMMKTHFGTDDIDSEDYKRKKKLSEEMLEYCGGLPLAITVLGGLLSRKPTADEWETLHKNIKTCIRKGKINEQEDSNFGVSWVLGLSYDELPYYLKPCFLHLAYFPEDFEIRARDLCLVWLAEGFVSKEERAYECLSELVERCIIQVAEWSSTGTIKTCRIHDLMRDLCLVKAEEEGFLQHADLRNHHEVTNFSSSSVGAATNLEPINKVRRLAIYLNNTSVDELVSLIRHKDGCLRSLICFNSEPDTSYEQVMKPLFNQFHLLRVLKFENYTHGHVGKLPKEIGNLIHLRLFSLKDSYVEIFPSSIGNLRCLQTLDLRVKNLYIDKQLQSSKLPPVLWKLEQLRHLYLPQKYLTEAERSKFTVTHNENILRLGNLTNLQTLVNVSVNYHYLNDLEKLTNLTKLKINLGSCFRGTRIKFINLRSLSICYDDIIIESEEYGTIRRGVEFSHHEIILLLLSCPQLHKLHLQVSMVKLPEENQFSSNLIKLTLLRTCLKDDPMSTLEKLPHLRILLLDHNAFLGQKMNCTSKGFPRLESLSISYLYDFIEWKVEEGALPSLRRLHIVNCLRLRTVPDGLRFVVTLKEIVIKHMLRQFKKKVEEGALPSLRRLHIVNCLRLRTVPDGLRFVVTLKEIVIKHMLRQFKKKVEEGGEDFYKVHHVPSLVFTNTLDF

SEQ ID NO: 39 - protein, 938 aa, Cannabis sativa
MADAVVSFVIERLGDLVISEAQFLGGVEAQLGNAQIKLQCMSAFLKDADAWVTNGNERVRLLVVQIRENSYALEDVIETYVLKVSSSHGTIKRALKGCVRIIDVHKVGSEIERISSNIDTWTSQLEALGVHRSIQKADEASSSSYVQQQRQLRQAYSFVEDNVVVGFDKDIEELVALLTEEENSHKHKVVSICGMGGLGKTTLARKVYQHSQVRTHFDCYAWASISQQCNTSNVFKGIHFSLTSPTDEQKIHIKNSSDVELARELFNFLKQKKCLVVLDDIWTTTTWDLLKHAFPTQGETHSKILLTTRNKDVALHADQHGFIHEPRLLNEEESWELFHKKYFCVGADPSNSNDDEERKKELAVEMLRKCSGLPLAIIVLAGLLSTKKKHTVYDWEQLKANVIRWIGEGGQQHDDNSKYISVRAVLGLSYSELPSHLKPCFLYLARYAEDVPIRVKDLCHILIAEGLISRRRGYVETWEEVAHDWLCELVERSMIQVKEMSSRGGRIKSFCIHDLMRDLCVSKAQEENFLHFIDWQNKGEEPIQTKVRRVAIYDNGKTVSSDFIHMVGNIDASLRCFAVDGGGNEYNEKRVLRHACNHFLKLRVLIIGYNSTYTLKLPKEIENMIHLRLLSIPNWPIEKIPSSFGNLRCLQSLRVRSKSDKIPSSMCKLEQLRHLYLITVDGNGIEFGKFLRSTKSRNLQTLVSIRTNDLVGSDLLQLKSLKKLEICVNGNFETFLHIPQSFTFSRLCSLKVESSNRYTAIDVVPLILSCPHIYKLRLYSAILRLPQVNLFSPNLIKLQLKFLRLKDDPMPTLEKLPKLRVLVIGYDSFMGDEMVCSRGGFPRLESLELSVLHNLKEWKVEESALATLADLRIHGCRGLRSVPDGIRNIVTLNEIKINNMPKKFKERMEEGGDDFHKVMHVPSRVFINCVRDWPDDYK

SEQ ID NO: 40 - protein, 961 aa, Cannabis sativa
MFLDHIISYNSIQFSSLTEKNHRMADAVVSFVIERLGDLVLSEAQFLGGVEAQVGNAQIKLQCMSAFLKDADAWVTNGNERVRLLVVQIRENSYALEDVIETYVLKVSSSHGTIKRALKGCVRIIDVHKVGSEIERISSNIDTWTSQLEALGVHRSIQKADEASSSSYVQQQRQLRQAYSFVEDNVVVGFDKDIEELVALLTEEENSHKHKVVSICGMGGLGKTTLARKVYQHSQVRTHFDCYAWASISQQCNTSNVFKGIHFSLTSPTDEQKIHIKNSSDVELARELFNFLKQKKCLVVLDDIWTTTTWDLLKHAFPTQGETHSKILLTTRNKDVALHADQHGFIHEPRLLNEEESWELFHKKYFCVGADPSNSNDDEERKKELAVEMLRKCSGLPLAIIVLAGLLSTKKKHTVYDWEQLKANVIRWIGEGGQQHDDNSKYISVRAVLGLSYSELPSHLKPCFLYLARYAEDVPIRVKDLCHILIAEGLISRRRGYVETWEEVAHDWLCELVERSMIQVKEMSSRGGRIKSFCIHDLMRDLCVSRAQEENFLHFTDWQNKGEEPIQTKVRRVAIYDNGKTVSSDFIHMVGNIDASLRCFAVDGGGNEYNEKRVLRHACNHFLKLRVLIIGYNSTYTLKLPKEIENMIHLRLLSIPNWPIEKIPSSFGNLRCLQSLRVRSKSDKIPSSMCKLEQLRHLYLITVDGNGIEFGKFLRSTKSRNLQTLVSIRTNDLVGSDLLQLKSLKKLEICVNGNFETFLHIPQSFTFSRLFSLKVESSNRYTAIDVVPLILSCPHIYKLRLYSAILRLPQVNLFSPNLIKLQLKFLRLKDDPMPTLEKLPKLRVLVIGYDSFMGDEMVCSRGGFPRLESLELSVLHNLKEWKVEESALATLADLRIHGCRGLRSVPDGIRNIVTLNEIKINNMPKKFKERMEEGGDDFHKVMHVPSRVFINCVRDWPDDYK

SEQ ID NO: 41 - protein, 975 aa, Cannabis sativa
NFLLLQQQKFQSHPKMAEGIVSFVIGRLGDLLINEANFLYGIRDQVEDAKSELQRIKCFLEDADARARHGDKSLRLHVAEIGEYAYDLEDVITNFVLKESSRVSISGSSSSSSSRMFSCGNFIEFHKVGSRIEKISARVSKSRLSFQSYGLVQSRGNDEAATNFSTRLEFRRTFSHNIDSDFVGFEENINELVEHLKKKGRNLHSVVSICGMGGLGKTTLARQVYYHGEIRRYFDCFAWASVSQKCQPRDVWEGILIRLTSPASERRREIKEMKDDEIAKALFKVCTEKRCLVVLDDIWSSSTWDCLKLAFPNVGSDSKIMLTTRNREVAFHADRNSILHEPRCFNKNETWELFMMKTHFGTDDIDSEDYKRKKKLAGEMLEYCGGLPLAITVLGGLLSRKPTADEWETLHKNIKTCIRKGKINEQEDSNFGVSWVLGLSYDELPYYLKPCFLHLAYFPEDFEIRARDLCLVWLAEGFVSKEERAYECLSELVERCIIQVAEWSSTGTIKTCRIHDLMRDLCLVKAEEEGFLQHADLRNHHEVTNFSSSSVGAATNLEPINKVRRLAIYLNNTSVDELVSLIRHKDGCLRSLICFNSEPDTSYEQVMKPLFNQFHLLRVLKFENYTHGHVGKLPKEIGNLIHLRLFSLKDSYVEIFPSSIGNLRCLQTLDLRVKNLYIDKQLQSSKLPPVLWKLEQLRHLYLPQKYLTEAERSKFTVTHNENILRLGNLTNLQTLVNVSVNYHYLNDLEKLTNLTKLKINLGSCFRGTRIKFINLRSLSICYDDIIIESEEYGTIRRGVEFSHHEIILLLLSCPQLHKLHLQVSMVKLPEENQFSSNLIKLTLLRTCLKDDPMATLEKLPHLRILLLDHNAFLGQKMNCTSKGFPRLESLSICYLYDFIEWKVEEGALPSLRRLHIVNCLRLRTVPDGLRFVVTLKEIVIKHMLRQFKKKVEEGGEDFYKVHHVPSLVFTNTLDF

SEQ ID NO: 42 - protein, 975 aa, Cannabis sativa; identical to SEQ ID NO: 41

SEQ ID NO: 43 - protein, 936 aa, Cannabis sativa
MADAVVSFVIERLGDLVISEAQFLGGVEAQVGNAQIKLQCMSAFLKDADAWVTNGNERVRLLVVQIRENSYALEDVIETYVLKVSSSHGTIKRALKGCVRIIDVHKVGSEIERISSNIDTWTSQLEALGVHRSIQKADEASSSSYVQQQRKLRQAYSFVEDNVVVGFDKDIEELVALLTEEENSHKHKVVSICGMGGLGKTTLARKVYQHSQVRTHFDCYAWASISQQCNTSNVFKGIHFSLTSPTDEQKIHIKNSSDVELARELFNFQKQKKCLVVLDDIWTTTTWDLLKHAFPTQGETHSKILLTTRNKDVALHADQHGFIHEPRLLNEEESWELFHKKYFCVEADPSNSNDDEERKKELAVEMLRKCSGLPLAIIVLAGLLSKKHTIYEWELVKRNVPRCIGQAEQQHDVDSNHHGVFGVLGLSYNELPSKLKPCFLYLARYAEDVPIRVKDLCHILIAEGLISRRRGYVETWEEVAHDWLCELVERSMIQVKEMSSRGGRIKSFCIHDLMRDLCVSKAQEENFLHFTDWQNKWEEPIKTKVRRVAIYDNGKTVSSDFIHMVGNIDASLRCFAVDGGGNEYNEKRVLRHACNHFLKLRVLIIGYNSIYTLKLPKEIENMIHLRLLSIPNWPIEKIPSSLGNLRCLQSLTVRSKSDKIPSSMCKLEQLRHLYLITVDGNGIEFGKFLRSTKSRNLQTLVSIRTNDLVGSDLLQLKSLKKLEICVNGNFETFLHIPQSFTFSRLCSLKVESSNRYTAIDVVPLILSCPHIYKLRLYSAILRLPQVNLFSPNLIKLQLKFLRLKDDPMPTLEKLPKLRVLVIGYDSFMGDEMVCSRGGFPRLESLELSVLHNLKEWKVEESALATLADLRIHGCRGLRSVPDGIRNIVTLNEIKINNMPKKFKERMEEGGDDFHKVMHVPSRVFINCVRDWPDDYK

SEQ ID NO: 44 - protein, 938 aa, Cannabis sativa
MADAVVSFVIERLGDLVISEAQFLGGVEAQLGNAQIKLQCMSAFLKDADAWVTNGNERVRLLVVQIRENSYALEDVIETYVLKVSSSHGTIKRALKGCVRIIDVHKVGSEIERISSNIDTWTSQLEALGVHRSIQKADEASSSSYVQQQRKLRQAYSFVEDNVVVGFDKDIEELVALLTEEENSHKHKVVSICGMGGLGKTTLARKVYQHSQVRTHFDCYAWASISQQCNTSNVFKGIHFSLTSPTDEQKIHIKNSSDVELARELFNFQKQKKCLVVLDDIWTTTTWDLLKHAFPTQGETHSKILLTTRNKDVALHADQHGFIHEPRLLNEEESWELFHKKYFCVEADPSNSNDDEERKKELAVEMLRKCSGLPLAIIVLAGLLSTKKKHTVYDWEQLKANVIRWIGEGGQQHDDNSKYISVRAVLGLSYSELPSHLKPCFLYLARYAEDVPIRVKDLCHILIAEGLISRRRGYVETWEEVAHDWLCELVERSMIQVKEMSSRGGRIKSFCIHDLMRDLCVSKAQEENFLHFTDWQNKWEEPIKTKVRRVAIYDNGKTVSSDFIHMVGNIDASLRCFAVDGGGNEYNEKRVLRHACNHFLKLRVLIIGYNSIYTLKLPKEIENMIHLRLLSIPNWPIEKIPSSLGNLRCLQSLRVRSKSDKIPSSMCKLEQLRHLYLITVDGNGIEFGKFLRSTKSRNLQTLVSIRTNDLVGSDLLQLKSLKKLEICVNGNFETFLHIPQSFTFSRLFSLKVESSNRYTAIDVVPLILSCPHIYKLRLYSAILRLPQVNLFSPNLIKLQLKFLRLKDDPMPTLEKLPKLRVLVIGYDSFMGDEMVCSRGGFPRLESLELSVLHNLKEWKVEESALATLADLRIHGCRGLRSVPDGIRNIVTLNEIKINNMPKKFKERMEEGGDDFHKVMHVPSRVFINCVRDWPDDYK

SEQ ID NO: 45 - protein, 963 aa, Cannabis sativa; identical to SEQ ID NO: 32

SEQ ID NO: 46 - protein, 960 aa, Cannabis sativa
MAEGIVSFVIGRLGDLLINEANFLYGIRDQVEDAKSELQRIKCFLEDADARARHGDKSLRLHVAEIGEYAYDLEDVITNFVLKESSRVSISGSSSSSSSRMFSCGNFIEFHKVGSRIEKISARVSKSRLSFQSYGLVQSRGNDEAATNFSTRLEFRRTFSHNIDSDFVGFEENINELVEHLKKKGRNLHSVVSICGMGGLGKTTLARQVYYHGEIRRYFDCFAWASVSQKCQPRDVWEGILIRLTSPASERRREIKEMKDDEIAKALFKVCTEKRCLVVLDDIWSSSTWDCLKLAFPNVGSDSKIMLTTRNREVAFHADRNSILHEPRCFNKNETWELFMMKTHFGTDDIDSEDYKRKKKLSEEMLEYCGGLPLAITVLGGLLSRKPTADEWETLHKNIKTCIRKGKINEQEDSNFGVSWVLGLSYDELPYYLKPCFLHLAYFPEDFEIRARDLCLVWLAEGFVSKEERAYECLSELVERCIIQVAEWSSTGTIKTCRIHDLMRDLCLVKAEEEGFLQHADLRNHHEVTNFSSSSVGAATNLEPINKVRRLAIYLNNTSVDELVSLIRHKDGCLRSLICFNSEPDTSYEQVMKPLFNQFHLLRVLKFENYTHGHVGKLPKEIGNLIHLRLFSLKDSYVEIFPSSIGNLRCLQTLDLRVKNLYIDKQLQSSKLPPVLWKLEQLRHLYLPQKYLTEAERSKFTVTHNENILRLGNLTNLQTLVNVSVNYHYLNDLEKLTNLTKLKINLGSCFRGTRIKFINLRSLSICYDDIIIESEEYGTIRRGVEFSHHEIILLLLSCPQLHKLHLQVSMVKLPEENQFSSNLIKLTLLRTCLKDDPMATLEKLPHLRILLLDHNAFLGQKMNCTSKGFPRLESLSICYLYDFIEWKVEEGALPSLRRLHIVNCLRLRTVPDGLRFVVTLKEIVIKHMLRQFKKKVEEGGEDFYKVHHVPSLVFTNTLDF

SEQ ID NO: 47 - protein, 960 aa, Cannabis sativa
MAEGIVSFVIGRLGDLLINEANFLYGIRDQVEDAKSELQRIKCFLEDADARARHGDKSLRLHVAEIGEYAYDLEDVITNFVLKESSRVSISGSSSSSSSRMFSCGNFIEFHKVGSRIEKISARVSKSRLSFQSYGLVQSRGNDEAATNFSTRLEFRRTFSHNIDSDFVGFEENINELVEHLKKKGRNLHSVVSICGMGGLGKTTLARQVYYHGEIRRYFDCFAWASVSQKCQPRDVWEGILIRLTSPASERRREIKEMKDDEIAKALFKVCTEKRCLVVLDDIWSSSTWDCLKLAFPNVGSDSKIMLTTRNREVAFHADRNSILHEPRCFNKNETWELFMMKTHFGTDDIDSEDYKRKKKLAGEMLEYCGGLPLAITVLGGLLSRKPTADEWETLHKNIKTCIRKGKINEQEDSNFGVSWVLGLSYDELPYYLKPCFLHLAYFPEDFEIRARDLCLVWLAEGFVSKEERAYECLSELVERCIIQVAEWSSTGTIKTCRIHDLMRDLCLVKAEEEGFLQHADLRNHHEVTNFSSSSVGAATNLEPINKVRRLAIYLNNTSVDELVSLIRHKDGCLRSLICFNSEPDTSYEQVMKPLFNQFHLLRVLKFENYTHGHVGKLPKEIGNLIHLRLFSLKDSYVEIFPSSIGNLRCLQTLDLRVKNLYIDKQLQSSKLPPVLWKLEQLRHLYLPQKYLTEAERSKFTVTHNENILRLGNLTNLQTLVNVSVNYHYLNDLEKLTNLTKLKINLGSCFRGTRIKFINLRSLSICYDDIIIESEEYGTIRRGVEFSHHEIILLLLSCPQLHKLHLQVSMVKLPEENQFSSNLIKLTLLRTCLKDDPMATLEKLPHLRILLLDHNAFLGQKMNCTSKGFPRLESLSICYLYDFIEWKVEEGALPSLRRLHIVNCLRLRTVPDGLRFVVTLKEIVIKHMLRQFKKKVEEGGEDFYKVHHVPSLVFTNTLDF

SEQ ID NO: 48 - protein, 955 aa, Cannabis sativa
MADAVVSFVIERIGDLLIDEAKFLYGVEGQVGNAKIKLQCMSAFLKDADAWVTNGNERVRLLVVQIRENSLDLEDVVETYVLKVALKGNEGSKRERFISIFREGIDVHKVGSEIERISSNIDTWTSQLEAFGVHRSIHNAAEASSSSHVQQQRQLRQAYSYVEDNYIVGFGKDIEELVTLLTEKENSRKHKVISVCGMGGLGKTTLARKVYQHPQVRTHFECYAWASISQQCNTRNVFEEVYFAFTSPTKERREEIKNLSDVELARELYNFRKQKKCLVVLDDIWTPTTWDLLQHAFPTTTQGDAHSKILLTTRNKVVALHADPHGFIHEPHLLNDKESWELFQNKCYSIGTDPSDSNDDEERKNELAVEMLKKCSGLPLAIIVLAGLLSKKHTLYDWEQLKANVIRWIGEGGQQHDDNSKYISVRAVLGLSYSELPSHLKPCFLYLARYAEDVPIRVKDLCHILIAEGLISRRRGYVETWEEVAHDWLCELVERSMIQVKEMSSRGGRIKSFCIHDLMRDLCVSKAQEENFLHFTDWQNKWEEPIKTKVRRVAIYDNGKTVSSDFIHMVGNIDASLRCFAVDGGGNEYNEKRVLRHACNHFLKLRVLIIGYNSIYTLKLPKEIENMIHLRLLSIPNWPIEKIPSSLGNLRCLQSLTVRSKSDKIPSSMYKLEQLRHLYLITVDGKGIEFGKFLRSTKSRNLQTLVSIRTNDLVGSDLLQLKSLKKLEICVNGNFETFLHNPQSLTFTRLFSLQVNNLFGVKIDIIPLILSCPHIYKLTLYSPILSLPQVNQFSPNLIKLTLRFLKLEDDPMPTLEKLPKLRVLMIGYDSFKGDEMVCSRGGFPRLESLELSVLHNLKEWKVEESALATLADLRIHGCRGLRSVPDGIRNIVTLNEIKIISMPKKFKERMEEGGDDFHKVNHVPSRVFINCDWDSNSEDAKLYKNLSGCGCGVWG

SEQ ID NO: 49 - protein, 958 aa, Cannabis sativa
MADAVVSFVIERIGDLLIDEAKFLYGVEGQVGNAKIKLQCMSAFLKDADAWVRNGDERVRLLVVQIRENSLDLEDVVETYVLKVALKGNEGSKRERFISIFREGIDVHKVGTEIERISSNIDTWTSQLEAFGVHRSIHNAAEASSSSHVQQQRQLRQAYSYVEDNYIVGFGKDIEELVTLLTEKENSRKHKVISVCGMGGLGKTTLARKVYQHPQVRTHFECYAWASISQQCNTRNVFEEVYFAFTSPTKERREEIKNLSDVELARELYNFRKQKKCLVVLDDIWTPTTWDLLQHAFPTTTQGDAHSKILLTTRNKVVALHADPHGFIHEPHLLNDKESWELFQNKCYSIGTDPSDSNDDEERKNELAVEMLKKCSGLPLAIIVLAGLLSKKHTLYDWEQLKANVIRCIGQGDQQHDVDSNYRGVSGVLGLSYSELPRHLKPCFLYLARYAEDVPIRVKELCLVLIAEGFISLRRGSVETLEEVAYDWLCELVERSMIQVKEMSSTEGRIKLFCIHDLMRDLCVSKAQEENFLHFTDWQNKGEQPIEANVRRVSIYDNSYRTIIDGSDFIHMFRNKDGSLRCLALHGGSTENEKRILRHACNHFLKLRVLIIGHQPVDIFSICLKLPKEIGNLIHLRLLSIPYWPIRKIPSSFGNLRCLQTLRVSTLSHNIPSSMCKLEQLRHLHFSDRGIKFGKFLRSTKSRNLQTLVGIDTEDVLLSDLLQLESLKKLGVEVYGNFETFLHNPQSLTFTRLFSLQVNNLFGVKIDIIPLILSCPHIYKLTLYSPILSLPQVNQFSPNLIKLTLRFLKLEDDPMPTLEKLPKLRVLMIGYDSFKGDEMVCSRGGFPRLESLQLIGLENLKEWKVEESALPRLAYLTIKWSWELRRVPDGVRNIVTLNEIKIISMPKKFKERMEEGGDDFHKVNHVPSRVFINCDWDSNSEDAKLYKNLSGCGCGVWG

SEQ ID NO: 50 - protein, 961 aa, Cannabis sativa
MAESLLISQLAEAVVSQAVGRLSELIINEGASLSSVKADVERLRDELHRMHCFFEDADRRPHHQDKRLRHWISELRGVANDVEDVIEMFISNVSSSCLQFFYLRRLRSQINSIKTKMEDIFKSSQTYRIESSSRSSSSRRVEGTSSSVSELQRRLRRSCPDDNDDDDYVVSMEHSVKALKDELIMLKKEDDDDQLSIVSVVGMGGLGKTTLAKLVYKSNDVKQHFDCCAWVFICQQYVSKDILYEILVQIDRGSDREKIRNLNENEMSDRLKSELKGKRYLVVLDDIWRIEAWDCIKRAFPLGNSSSKVLFTTRNKELALSVNPCSSPIEPSFLTFEESWQVLVQRQAFRRSGFGLEFEMLGKDMVKKCGGLPLAIVVLGGLLRTRSSLEEWKEVQKDVNSHLNKLQSHQEYEGVNGILALSYYDLPYYLKPCFLYLGSFPEDRDIEKKTLIRLWIAEGFVQKPKREEANEETLENVCEQYLRELTNRCMIQVSKRNNSGVGVKTYRVHDLMRDLCVSKAKEENFFQVIQHQNESNMSEQGGSSIHHGAKTIFSRKIVLHVGCDLDLCKVQLRLHSLLCFDNGHFLVSSLRKKTFMFLRTLILGNTCLGDNNFKILKQICTLIHLRYLGLQKSNNLHKLPKCIANLRSLHTLDLRYSNVKMLPTSISRLTRLTHLMSDKPLMLMDKESGNKFLNFAVLSVNPAKPTPTNPAQPTTPMLIRDVLIFKYDVLLQLTNLQELKMLLRSKEEATKVLESAIIKGGRIRSLDLDMVFHGHVLQNQLAPPPPMPFPSLKSLSKCYLLNKLRLRGPIEKDSLQFLSTSLVKLTLEHTCIQQDPMAVLEQLPNLKLLQLHSFSYIGSKLVCSANGFPKLETLRLCHLQRLKEWEIEKDSMQCLKRLELEQLLKLKMIPDGLKFVTTLRQLDVIEMTQIFEERIKVSEGLEGEDYNKVRHIPSISTRRLY

SEQ ID NO: 51 - protein, 939 aa, Cannabis sativa
MADAVVSFVIERLGDLVISEAQFLGGVEAQVGNAQIKLQCMSAFLKDADAWVRNGDERVRLLVVQVRENAYALEDVIETYVLKVTLKKNEGVLKRSINIFREGIDVHKVGSEIERISSNIDTWTSQLEALGVHRSIQKADEASSSSYVQQQRKLRQAYSFVEDNVVVGFDKDIKDLVGLLTEKENPHKHKVISVCGMGGLGKTTLAKKVYQHPQVRTHFDCYAWASISQQCNRRDVFEEVYFAFTSPTDAQRKRIKNLSDVELARELYNFQKQKKCLVVLDDIWTATTWDLLQHAFPTTTQGDTLHSKILLTTRNKDVALHADQHGFIHEPHLLNEEESWELFHKKYFSVGADPSNSNYDEERKKELAVEMLRKCSGLPLAIIVLAGLLSKKHTLYDWEQLKANVIRWIGQGAQQHYDDSKYRSVRGVLGLSYSELPRHLKPCFLYLARYAEDVPIRAKELYLVLIAEGFISRRGGSVETLEEVAYDWLCELVERSMIQVKEMSLTEGRIKSFCIHDLMRDLCVSKAQEENFLHFTDWQNKGEQPIEANVRRVSIYDNSYSIIDGSDFIHMFRNKDGSLRCLALYCERIEKRKRILRHACNHFLKLRVLIIGDRYLFKTLKLPKEIGNLIHLRLLSIPNWEIKNIPSSFGNLRCLQTLRVWTRSYKIPSSMWKLEQLRHLYFTPVGIEFGNFLRSTKSRNLQTLVGIRTKDLLLSDLLQLESLKKLGIRVDRNFDRFLHKPQTLTFTRLQSLQMNNRFGTKIDIVPLILSCPHIYKLRVELPIIRLPEVNQFSPNLIKLRLFDLRLEDDPMPTLEKLPKLRVLVIGYDSFMGDEMVCSRGGFPRLESLELSVLHNLKEWKVEESALPTLAYLRIYCCRKLRRVPDGVRNIVTLNEMVIDYMPKKFKERMEEGGDDFHKVNHVPSRVFMECDTDWLPEDD

SEQ ID NO: 52 - protein, 939 aa, Cannabis sativa
MADAVVSFVIERLGDLVISEAQFLGGVEAQVGNAQIKLQCMSAFLKDADAWVRNGDERVRLLVVQVRENAYALEDVIETYVLKVTLKKNEGVLKRSINIFREGIDVHKVGSEIERISSNIDTWTSQLEALGVHRSIQKADEASSSSYVQQQRKLRQAYSFVEDNVVVGFDKDIKDLVGLLTEKENPHKHKVISVCGMGGLGKTTLAKKVYQHPQVRTHFDCYAWASISQQCNRRDVFEEVYFAFTSPTDAQRKRIKNLSDVELARELYNFQKQKKCLVVLDDIWTATTWDLLQHAFPTTTQGDTLHSKILLTTRNKDVALHADQHGFIHEPHLLNEEESWELFHKKYFSVGADPSNSNYDEERKKELAVEMLRKCSGLPLAIIVLAGLLSKKHTLYDWEQLKANVIRWIGQGAQQHYDDSKYRSVRGVLGLSYSELPRHLKPCFLYLARYAEDVPIRAKELYLVLIAEGFISRRGGSVETLEEVAYDWLCELVERSMIQVKEMSLTEGRIKSFCIHDLMRDLCVSKAQEENFLHFTDWQNKGEQPIEANVRRVSIYDNSYSIIDGSDFIHMFRNKDGSLRCLALYCERIEKRKRILRHACNHFLKLRVLIIGDRYLFKTLKLPKEIGNLIHLRLLSIPNWEIKNIPSSFGNLRCLQTLRVWTRSYKIPSSMWKLEQLRHLYFTPVGIEFGNFLRSTKSRNLQTLVGIRTKDLLLSDLLQLESLKKLGIRVDRNFDRFLHKPQTLTFTRLQSLQMNNRFGTKIDIVPLILSCPHIYKLRVELPIIRLPEVNQFSPNLIKLRLFDLRLEDDPMPTLEKLPKLRVLVIGYDSFMGDEMVCSRGGFPRLESLRLISLENLKEWKVEESALPTLAYLRIYCCRKLRRVPDGVRNIVTLNEMVIDYMPKKFKERMEEGGDDFHKVNHVPSRVFMECDTDWLPEDD

SEQ ID NO: 53 - protein, 939 aa, Cannabis sativa
MADAVVSFVIERLGDLVISEAQFLGGVEAQVGNAQIKLQCMSAFLKDADAWVRNGDERVRLLVVQIREKSLDLEDVIETYVLKVTLRRNEGLLKRCICIFRKGIDVHKVGSEIERISSDIHTWTSQLEAFGVRRSIHNAAEASSSSYVQQQRKLRQAYSFVEDNVVVGFDKDIKDLVGLLTEKENPHKHKVISVCGMGGLGKTTLAKKVYQHPQVRTHFDCYAWASISQQCNRRDVFEEVYFAFTSPTDAQRKRIKNLSDVELARELYNFQKQKKCLVVLDDIWTATTWDLLQHAFPTTTQGDTLHSKILLTTRNKDVALHADQHGFIHEPHLLNEEESWELFHKKYFSVGADPSNSNYDEERKKELAVEMLRKCSGLPLAIIVLAGLLSKKHTLYDWEQLKANVIRWIGQGAQQHYDDSKYRSVRGVLGLSYSELPRHLKPCFLYLARYAEDVPIRAKELYLVLIAEGFISRRGGSVETLEEVAYDWLCELVERSMIQVKEMSLTEGRIKSFCIHDLMRDLCVSKAQEENFLHFTDWQNKGEQPIEANVRRVSIYDNSYSIIDGSDFIHMFRNKDGSLRCLALYCERIEKRKRILRHACNHFLKLRVLIIGDRYLFKTLKLPKEIGNLIHLRLLSIPNWEIKNIPSSFGNLRCLQTLRVWTRSYKIPSSMWKLEQLRHLYFTPVGIEFGNFLRSTKSRNLQTLVGIRTKDLLLSDLLQLESLKKLGIRVDRNFDRFLHKPQTLTFTRLQSLQMNNRFGTKIDIVPLILSCPHIYKLRVELPIIRLPEVNQFSPNLIKLRLFDLRLEDDPMPTLEKLPKLRVLVIGYDSFMGDEMVCSRGGFPRLESLRLISLENLKEWKVEESALPTLAYLRIYCCRKLRRVPDGVRNIVTLNEMVIDYMPKKFKERMEEGGDDFHKVNHVPSRVFMECDTDWLPEDD

SEQ ID NO: 54 - protein, 947 aa, Cannabis sativa
MADAVVSFVIERLGDLVISEAQFLGGVEAQVGNAQIKLQCMSAFLKDADAWVRNGDERVRLLVVQIREKSLDLEDVIETYVLKVTLRRNEGLLKRCICIFRKGIDVHKVGSEIERISSDIHTWTSQLEAFGVRRSIHNAAEASSSSYVQQQRKLRQAYSFVEDNVVVGFDKDIKDLVGLLTEKENPHKHKVISVCGMGGLGKTTLAKKVYQHPQVRTHFDCYAWASISQQCNRRDVFEEVYFAFTSPTDAQRKRIKNLSDVELARELYNFQKQKKCLVVLDDIWTATTWDLLQHAFPTTTQGDTLHSKILLTTRNKDVALHADQHGFIHEPHLLNEEESWELFHKKYFSVGADPSNSNYDEERKKELAVEMLRKCSGLPLAIIVLAGLLSKKHTLYDWEQLKANVIRWIGQGAQQHYDDSKYRSVRGVLGLSYSELPRHLKPCFLYLARYAEDVPIRAKELYLVLIAEGFISRRGGSVETLEEVAYDWLCELVERSMIQVKEMSLTEGRIKSFCIHDLMRDLCVSKAQEENFLHFTDWQNKGEQPIEANVRRVSIYDNSYSIIDGSDFIHMFRNKDGSLRCLALYCERIEKRKRILRHACNHFLKLRVLIIGDRYLFKTLKLPKEIGNLIHLRLLSIPNWEIKNIPSSFGNLRCLQTLRVWTRSYKIPSSMWKLEQLRHLYFTPVGIEFGNFLRSTKSRNLQTLVGIRTKDLLLSDLLQLESLKKLGIRVDRNFDRFLHKPQTLTFTRLQSLQMNNRFGTKIDIVPLILSCPHIYKLRVELPIIRLPEVNQFSPNLIKLRLFDLRLEDDPMPTLEKLPKLRVLVIGYDSFMGDEMVCSRGGFPRLESLRLISLENLKEWKVEESALPTLAYLRIYCCRKLRRVPDGVRNIVTLNEMVIDYMPKKFKERMEEGGDDFDQVKRVPSRVFINCDQESEDSVNAIYNQLNL

SEQ ID NO: 55 - protein, 937 aa, Cannabis sativa
MADAVVSFVIERLGDLVISEAQFLGGVEAQVGNAQIKLQCMSAFLKDADAWVTNGDERVRLLVVQIREKSLDLEDVIETYVLKVTLRRNEGLLKRCICIFRKGIDVHKVGSEIERISSDIHTWTSQLEAFGVRRSIHNAAEASSSSYVQQQRKLRQAYSFVEDNVVVGFDKDIKDLVGLLTEKENPHKHKVISVCGMGGLGKTTLAKKVYQHPQVRTHFDCYAWASISQQCNRRDVFEEVYFAFTSPTDAQRKRIKNLSDVELARELYNFQKQKKCLVVLDDIWTATTWDLLQHAFPTTTQGDTLHSKILLTTRNKDVALHADQHGFIHEPHLLNEEESWELFHKKYFSVGADPSNSNYDEERKKELAVEMLRKCSGLPLAIIVLAGLLSKKHTLYDWEQLKANVIRWIGQGAQQHYDDSKYRSVRGVLGLSYSELPRHLKPCFLYLARYAEDVPIRAKELYLVLIAEGFISRRGGSVETLEEVAYDWLCELVERSMIQVKEMSLTEGRIKSFCIHDLMRDLCVSKAQEENFLHFTDWQNKGEQPIEANVRRVSIYDNGNIDGSDFIHMFRNKDGSLRCLALYCERIEKRKRILRHACNHFLKLRVLIIGDRYLFKTLKLPKEIGNLIHLRLLSIPYWPIRKIPSSFGNLRCLQTLRVWTRSYKIPSSMWKLEQLRHLYFTPVGIEFGNFLRSTKSRNLQTLVGIRTKDLLLSDLLQLESLKKLGIRVDRNFDRFLHKPQTLTFTRLQSLQMNNRFGTKIDIVPLILSCPHIYKLRVNLSIVRLPQVNQFSPNLIKLRLFDLRLEDDPMPTLEKLPKLRVLEIGVDSFRGDEMVCSRGGFPRLESLRLISLENLKEWKVEESALPTLAYLRIYCCRKLRRVPDGVRNIVTLNEIKIICMPKKFKERMEEGGDDFHKVNHVPSRVFMECDTDWLPEDD

SEQ ID NO: 56 - protein, 901 aa, Cannabis sativa
MAEYVVPLVVEKLASQLMEEAISLSKGKEHVEWIEAELRRMQCFLKDADAKQDKDARISNWVVDIREVAFDADDFIDTYIYKTNGQREKGLVMCFKRYSHLFFLCELVSNFKINKNISRIKSKIHEIADSKLTYGIENIRQKSEGATLTVSELQERRISSPHHFEDDIIGLDEDIKILESQLIYGQSCRSVLSIIGMAGLGKTTLAKMIYHSNGIKNNFDCCAWIYVSQEYSAEEVLRDLAKKVMGFSKIELEKMHREDLVEEISKFLQRRRYVIVLDDIWKKQVWDNLKDAFPDSKNGSRIMLTTRFKDIAIHADPRSPPHEPSLLNDEESWQLLSRKISLQWNAVTSLPPWTDELGKKIVKKCGGLPLAIVVLGGLLSRREASYSEWLKVLQSVHWQLRQDTIQCADILALSYFDLPFYLKPCFLYFGLFPEDFEISASRLILLWVAEGFVQPRGQEPIEDVSEDYLEELVGRSMVQVATRNSNGRIKTCRMHDLLRELAIAKATEDRFLHVIHGESRAGSIRTLSRSRRLAIHFNFPPSKKTTMNLRSLLCFDLNEPVKVQVKRFKLLRVLDLEGSYISKLDSAIGKLIHLRYLGLRGTWLKNLPRSLRYLSNLETLDLRSTLINPFPDEILRLQQLRHLYFSDVQPMVSIPPTIPSTYLPDLQTLKGLFISEISRSDIEDFLSKLTTMRSLSLHGRLCIKKEHLGNWVAKLKSLECLKLNATRLMTEDTIHAAVPEWDLSCLTHLYKIHLAGSMQKMFDIQYFPTNLSELTLTQSWLMEDPMKTLGKLRSLRVLKLKHAAYVGREITCSSGGFVQLQLLKLSFMNSVESLRIEEGAMPNLRELEIVQCKLKIVPRGLWPVQSLKSLKLGFMPGHDIGLKIQEREGENWYRIEHVLPI

SEQ ID NO: 57 - DNA, 22 bp, synthetic construct
  source 1..22: mol_type = other DNA
tgaggatgatccaatgccaaca

SEQ ID NO: 58 - DNA, 23 bp, synthetic construct
  source 1..23: mol_type = other DNA
acgacaatgttcatgagacaaca

SEQ ID NO: 59 - DNA, 31 bp, synthetic construct
  source 1..31: mol_type = other DNA
tcatccatctatcttgtgttatttcattgct

SEQ ID NO: 60 - DNA, 25 bp, synthetic construct
  source 1..25: mol_type = other DNA
acactatcctctgattctgcattca

SEQ ID NO: 61 - DNA, 20 bp, synthetic construct
  source 1..20: mol_type = other DNA
tgcattttctgtgttggcat

SEQ ID NO: 62 - DNA, 20 bp, synthetic construct
  source 1..20: mol_type = other DNA
tgcattttctgtattggcat

(19) United States
(12) Patent Application Publication
(10) Pub. No.: US 2026/0344605 A1
(43) Pub. Date: Nov. 5, 2026

(54) POWDERY MILDEW RESISTANT CANNABIS

(71) Applicant: Dewey Scientific, Pullman, WA (US)

(72) Inventors: Jordan ZAGER, Pullman, WA (US); Paul MIHALYOV, Pullman, WA (US)

(73) Assignee: Dewey Scientific, Pullman, WA (US)

(21) Appl. No.: 19/106,146

(22) PCT Filed: Aug. 24, 2023
(86) PCT No.: PCT/US2023/072827

§ 371 Date: Feb. 24, 2025

Related U.S. Application Data
(60) Provisional application No. 63/373,540, filed on Aug. 25, 2022.

(51) Int. Cl. A01H 6/28; A01H 1/00; A01H 1/02; A01H 1/04; A01H 5/02

(52) U.S. Cl. CPC A01H 6/28; A01H 1/021; A01H 1/045; A01H 1/101; A01H 1/1215; A01H 1/1255; A01H 5/02

(57) ABSTRACT

The present disclosure provides an isolated Cannabis sativa L. plant that is resistant to powdery mildew and has elevated levels of tetrahydrocannabinolic acid. Resistance is conferred by powdery mildew resistance genes; transcripts and proteins produced from powdery mildew resistance genes are provided. Also provided are isolated powdery mildew resistant plants with increased yields, increased lateral branching, increased flower site stacking, and/or increased number of top of the canopy flowers. Methods for providing and selecting a Cannabis sativa L. plant that is resistant to powdery mildew are further provided.

### CROSS-REFERENCE TO RELATED APPLICATIONS

This application claims priority to U.S. Provisional Patent Application No. 63/373,540, filed on Aug. 25, 2022, the entirety of which is incorporated herein by reference.

### REFERENCE TO A SEQUENCE LISTING

The contents of the electronic sequence listing (227352000140SEQLIST.xml; Size: 161,436 bytes; and Date of Creation: Aug. 23, 2023) are herein incorporated by reference in its entirety.

### FIELD OF THE INVENTION

The present invention relates to powdery mildew resistance genes in Cannabis sativa L. and especially to powdery mildew resistant Cannabis sativa L. plants. The present invention further relates to methods for obtaining the present powdery mildew resistant Cannabis sativa L. plants and the use of the present genes for providing powdery mildew resistance in Cannabis sativa L.

### BACKGROUND

Emerging nutraceutical markets have driven increased agricultural interest in Cannabis sativa, commonly known as hemp or marijuana, due to its suite of secondary metabolites including the cannabinoids tetrahydrocannabinolic acid (THCA) and cannabidiolic acid (CBDA). Among the many research focuses deserving attention, one priority should be identifying genetic markers and genes linked to naturally occurring disease resistance, thereby accelerating the development of varieties which are less dependent on the use of pesticides in disease management. The present invention addresses the widespread need for collaborative germplasm improvement strategies, including fast routes (single-cross) for mapping genetic resistance to powdery mildew, one of the most prevalent fungal diseases in cannabis.

Powdery mildew is a common term for several taxa of plant pathogenic fungi, including fungi from the genus Golovinomyces, and has been reported on both marijuana and industrial hemp-type C. sativa. Powdery mildew represents a significant limitation to cannabis production, especially in greenhouse systems. Symptoms of powdery mildew include patchy white mycelial colonies that develop on foliage and flowers, contributing to reduced plant vigor. Infection can result in a reduction of end-use quality. The economic impact of powdery mildew on cannabis production is significant.

Fungicides may be used to manage powdery mildew, but are accompanied by uncertainties regarding compliance, efficacy, and consumer response. Regulatory agencies throughout the world have been slow to adopt new guidelines for the use of agrochemicals on cannabis, creating a backlog of research needed to safely apply pesticides. Furthermore, efficacy data for the use of pesticides against powdery mildew in cannabis is lacking. Alternative strategies to mitigate powdery mildew include strategic environmental control, the use of high-powered UV-C lamps, and application of plant-growth promoting rhizobacteria, yet powdery mildew continues to represent one of the most prominent biological diseases in both field and greenhouse settings. Thus, the identification and characterization of powdery mildew resistance genes are of vital importance to the growth and sustainability of the cannabis industry.

### BRIEF SUMMARY

The present application provides an isolated Cannabis sativa L. plant that is resistant to powdery mildew, wherein the plant comprises one or more powdery mildew resistance genes, and wherein the plant produces flowers with greater than 22% tetrahydrocannabinolic acid (THCA) (wt/wt). In some embodiments, the isolated plant produces flowers with greater than 23%, 24%, or 25% THCA (wt/wt).

Several embodiments relate to the isolated plant comprising one or more powdery mildew resistance genes. In some embodiments, the resistance gene is located on Chromosome 2 of GenBank assembly accession no. GCA_900626175.2. In some embodiments, the resistance gene encodes a protein in the NB-ARC-LRR family. In some embodiments, the resistance gene encodes a transcript comprising a polynucleotide at least 75% identical, 80% identical, 85% identical, 90% identical, 95% identical, 96% identical, 97% identical, 98% identical, or 99% identical to a polynucleotide selected from the group consisting of SEQ ID NOs: 1-28. In some embodiments, the one or more resistance gene encodes one or more transcripts comprising a polynucleotide selected from the group consisting of SEQ ID NOs: 1-28. In some embodiments, the resistance gene encodes a transcript comprising a polynucleotide at least 75% identical, 80% identical, 85% identical, 90% identical, 95% identical, 96% identical, 97% identical, 98% identical, or 99% identical to a polynucleotide selected from the group consisting of SEQ ID NO: 6 and SEQ ID NO:27. In some embodiments, the resistance gene encodes a transcript comprising SEQ ID NO: 6. In some embodiments, the resistance gene encodes a transcript comprising SEQ ID NO: 27. In some embodiments, the one or more resistance genes encodes one or more proteins comprising at least one polypeptide that is at least 70%, at least 75%, at least 80%, at least 85%, at least 90%, at least 95%, at least 96%, at least 97%, at least 98%, at least 99%, or 100% identical to a polypeptide selected from the group consisting of SEQ ID NOs: 29-56. In some embodiments, the resistance gene encodes a protein comprising a polypeptide selected from the group consisting of SEQ ID NOs: 29-56. In some embodiments, the resistance gene encodes a protein comprising a polypeptide at least 75% identical, 80% identical, 85% identical, 90% identical, 95% identical, 96% identical, 97% identical, 98% identical, or 99% identical to a polypeptide selected from the group consisting of SEQ ID NO: 34 and SEQ ID NO: 55. In some embodiments, the resistance gene encodes a protein comprising a polypeptide selected from the group consisting of SEQ ID NOs: 34 and 55. In some embodiments, the resistance gene encodes a protein comprising a polypeptide of SEQ ID NO: 34. In some embodiments, the resistance gene encodes a protein comprising a polypeptide of SEQ ID NO: 55. In some embodiments, the plant comprises more than one resistance gene. In some embodiments, the plant comprises two, three, or four resistance genes. In some embodiments, the resistance gene allele is a dominant allele. In some embodiments, the plant is homozygous for the resistance gene allele. In some embodiments, the plant is heterozygous for the resistance gene allele. In some embodiments, the plant is resistant to infection by fungi of the genus Golovinomyces. In some embodiments, the plant is resistant to infection by fungi of the species Golovinomyces ambrosiae.

In some embodiments, the plant produces at least 41 g per plant of harvestable floral material. In some embodiments, the plant produces at least 42 g, 43 g, 44 g, 45 g, 46 g, or 47 g per plant of harvestable floral material. In some embodiments, the harvestable floral material comprises flowers and/or flower buds. In some embodiments, the plant produces harvestable material comprising flowers and/or flower buds, leaves, stalks, and seeds. In some embodiments, a plant part, tissue, or cell of the isolated Cannabis sativa L. plant is claimed. In some embodiments, the plant cell is a non-regenerable cell. In some embodiments, the isolated plant part further comprises harvestable material comprising flowers and/or flower buds, leaves, stalks, and seeds. In some embodiments, the plant has increased lateral branching as compared to a control Cannabis sativa L. plant. In some embodiments, the plant has at least 5%, at least 10%, at least 15%, at least 20%, at least 25%, at least 30%, at least 35%, at least 40%, at least 45%, or at least 50% more lateral branches as compared to a control Cannabis sativa L. plant. In some embodiments, the isolated powdery mildew resistant Cannabis sativa L. plant has an increased number of top of the canopy flowers as compared to a control Cannabis sativa L. plant. In some embodiments, the isolated powdery mildew resistant Cannabis sativa L. plant has at least 5%, at least 10%, at least 15%, at least 20%, at least 25%, at least 30%, at least 35%, at least 40%, at least 45%, or at least 50% more top of the canopy flowers as compared to a control Cannabis sativa L. plant. In some embodiments, the isolated powdery mildew resistant Cannabis sativa L. plant has an increased number of nodes when compared to a control Cannabis sativa L. plant. In some embodiments, the plant has increased flower site stacking as compared to a control Cannabis sativa L. plant. In some embodiments, the control Cannabis sativa L. plant is a naturally-occurring powdery mildew resistant plant. In some embodiments, the control Cannabis sativa L. plant is from the ‘PNW39’ population of plants.

Several embodiments relate to a method of selecting a Cannabis sativa L. plant that is resistant to powdery mildew, comprising crossing a first Cannabis sativa L. plant resistant to powdery mildew to a second Cannabis sativa L. plant to produce a population of offspring Cannabis sativa L. plants; genotyping the offspring population of Cannabis sativa L. plants for the presence of one or more powdery mildew resistance genes; and selecting an offspring Cannabis sativa L. plant on the basis of the genotyping, wherein the selected offspring Cannabis sativa L. plant is resistant to powdery mildew. In some embodiments, the selected offspring Cannabis sativa L. plant produces flowers with greater than 22% tetrahydrocannabinolic acid (THCA) (wt/wt). In some embodiments, the selected offspring Cannabis sativa L. plant produces flowers with greater than 23%, 24%, or 25% THCA (wt/wt). In some embodiments, the resistance gene is located on Chromosome 2 of GenBank assembly accession no. GCA_900626175.2. In some embodiments, the resistance gene encodes a protein in the NB-ARC-LRR family. In some embodiments, the resistance gene encodes a transcript comprising a polynucleotide at least 75% identical, 80% identical, 85% identical, 90% identical, 95% identical, 96% identical, 97% identical, 98% identical, or 99% identical to a polynucleotide selected from the group consisting of SEQ ID NOs: 1-28. In some embodiments, the resistance gene encodes a transcript comprising a polynucleotide selected from the group consisting of SEQ ID NOs: 1-28. In some embodiments, the resistance gene encodes a transcript comprising a polynucleotide at least 75% identical, 80% identical, 85% identical, 90% identical, 95% identical, 96% identical, 97% identical, 98% identical, or 99% identical to a polynucleotide selected from the group consisting of SEQ ID NO: 6 and SEQ ID NO: 27. In some embodiments, the resistance gene encodes a transcript comprising a polynucleotide selected from the group consisting of SEQ ID NO: 6 and SEQ ID NO: 27. In some embodiments, the resistance gene encodes a transcript comprising SEQ ID NO: 6. In some embodiments, the resistance gene encodes a transcript comprising SEQ ID NO: 27. In some embodiments, the one or more resistance genes encodes one or more proteins comprising at least one polypeptide that is at least 70%, at least 75%, at least 80%, at least 85%, at least 90%, at least 95%, at least 96%, at least 97%, at least 98%, at least 99%, or 100% identical to one or more of SEQ ID NOs: 29-56. In some embodiments, the resistance gene encodes a protein comprising a polypeptide selected from the group consisting of SEQ ID NOs: 29-56. In some embodiments, the resistance gene encodes a protein comprising a polypeptide at least 75% identical, 80% identical, 85% identical, 90% identical, 95% identical, 96% identical, 97% identical, 98% identical, or 99% identical to a polypeptide selected from the group consisting of SEQ ID NO: 34 and SEQ ID NO:55. In some embodiments, the resistance gene encodes a protein comprising a polypeptide selected from the group consisting of SEQ ID NO: 34 and SEQ ID NO: 55. In some embodiments, the resistance gene encodes a protein comprising a polypeptide of SEQ ID NO: 34. In some embodiments, the resistance gene encodes a protein comprising a polypeptide of SEQ ID NO: 55. In some embodiments, the second Cannabis sativa L. plant is not resistant to powdery mildew. In some embodiments, the selected offspring Cannabis sativa L. plant produces at least 41 g per plant of harvestable floral material. In some embodiments, the selected offspring Cannabis sativa L. plant produces at least 42 g, 43 g, 44 g, 45 g, 46 g, or 47 g per plant of harvestable floral material. In some embodiments, the selected offspring Cannabis sativa L. plant has increased lateral branching as compared to the first Cannabis sativa L. plant. In some embodiments, the selected offspring Cannabis sativa L. plant has at least 5%, at least 10%, at least 15%, at least 20%, at least 25%, at least 30%, at least 35%, at least 40%, at least 45%, or at least 50% more top of the canopy flowers as compared to the first Cannabis sativa L. plant. In some embodiments, the selected offspring Cannabis sativa L. plant has increased flower site stacking as compared to the first Cannabis sativa L. plant.

Also provided herein is a plant selected by the method of any of the previous embodiments or combination of previous embodiments.

Other aspects and specific embodiments are disclosed in the following detailed description and working Examples.

### DESCRIPTION OF THE FIGURES

The present application can be understood by reference to the following description taken in conjunction with the accompanying figures.

FIG. 1 shows susceptible and resistant phenotypes revealed by experiments with heavy disease pressure imposed through plant maturity. The left image shows a susceptible phenotype with dense mycelial growth (pictured: individual #45 from the population “PNW39”×“Jumping Jack”). The right image shows a resistant phenotype absent of any powdery mildew colonies (pictured: individual #49 from the population “PNW39”×“Jumping Jack”).

FIG. 2 shows a linkage map describing locations of selected markers on chromosome 2 (based on “CBDRx” chromosome naming). Resistance gene PM1 co-localizes with SNP markers LH3804, LH31156, and LH17304 on chromosome 2.

FIGS. 3A-3B show allelic discrimination plots from SNP assay PM_LH3804. Black dots represent individuals with the susceptible phenotype and gray dots represent individuals with the resistant phenotype. FIG. 3A shows an allelic discrimination plot using the parents and 89 progeny from “PNW39”×“Jumping Jack”. FIG. 3B shows an allelic discrimination plot using 96 F1 progeny from “PNW39”×“Dwy1337”.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

The present disclosure relates to isolated Cannabis sativa L. plants that are resistant to infection with powdery mildew.

As used in this specification and the appended claims, the singular forms ‘a’, ‘an’ and ‘the’ include plural referents unless the content clearly dictates otherwise. Thus, for example, reference to “a molecule” optionally includes a combination of two or more such molecules, and the like.

The term ‘and/or’ where used herein is to be taken as specific disclosure of each of the two specified features or components with or without the other. Thus, the term ‘and/or’ as used in a phrase such as ‘A and/or B’ herein is intended to include ‘A and B,’ ‘A or B,’ ‘A’ (alone), and ‘B’ (alone). Likewise, the term ‘and/or’ as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

An ‘isolated plant’, as used herein, describes a plant that is not a naturally-occurring strain or ecotype, but was deliberately bred with human intervention.

‘Powdery mildew’, as used herein, is a fungal disease that is capable of infecting numerous species of plants, including Cannabis sativa L. Infection results in a layer of white mildew growing on the surface of the leaves of the plant, and may inhibit growth of the plant as well as reduce yield and quality of harvestable material. Powdery mildew is caused by infection with fungi of the family Erysiphaceae, including the following genera: Arthrocladiella, Blumeria, Brasiliomyces Bulbomicrosphaera, Bulbouncinula, Caespitotheca, Cystotheca, Erysiphe (Oidium), Golovinomyces, Leveillula (Oidiopsis), Medusosphaera, Microsphaera, Neoerysiphe, Oidium, Phyllactinia (Ovulariopsis), Pleochaeta, Podosphaera, Sawadaea, Setoerysiphe, Sphaerotheca, Typhulochaeta, Uncinula, and Uncinuliella. Powdery mildew on Cannabis has been reportedly caused by infection with fungi of the genera Podosphaera and Golovinomyces (see Ocamb, “Hemp (Cannabis sativa)-Powdery Mildew”, Pacific Northwest Plant Disease Management Handbook, revised March 2023).

Within the genus Golovinomyces, infection could be caused by the following species: G. adenophorae, G. ambrosiae, G. americanus, G. andinus, G. arabidis, G. artemisiae, G. asperifolii, G. asperifoliorum, G. asterum, G. biocellatus, G. bolayi, G. brunneopunctatus, G. calceolariae, G. californicus, G. caulicola, G. chrysanthemi, G. cichoracearum, G. clematidis, G. cucurbitacearum, G. cynoglossi, G. depressus, G. echinopis, G. euphorbiicola, G. fischeri, G. franseriae, G. glandulariae, G. greeneanus, G. hydrophyllacearum, G. hyoscyami, G. immersus, G. inulae, G. laporteae, G. latisporus, G. longipes, G. lycopersici, G. macrocarpus, G. magnicellulatus, G. monardae, G. montagnei, G. neosalviae, G. ocimi, G. orontii, G. poonaensis, G. prenanthis, G. pseudosepultus, G. riedlianus, G. robustus, G. rogersonii, G. rubiae, G. salvia, G. senecionis, G. simplex, G. sonchicola, G. sordidus, G. spadiceus, G. sparsus, G. tabaci, G. valerianae, G. verbasci, G. verbenae, and G. vincae.

A plant that is ‘resistant to powdery mildew’, as used herein, identifies a plant that will not be infected with powdery mildew, even when exposed to high disease pressure, i.e., when grown surrounded by plants that are infected with powdery mildew.

A ‘powdery mildew resistance gene’, as used herein, identifies a gene or an allele of a gene that confers resistance to infection with powdery mildew to a plant.

A ‘dominant’ allele, as used herein, identifies an allele of a gene that confers a given phenotype when one or more copies of the allele are present in the organism. For example, a dominant powdery mildew resistance allele confers resistance to powdery mildew to an organism when the genome of the organism comprises one or more copies of the dominant powdery mildew resistance allele.

The term ‘heterozygous’, as used herein, identifies a locus in the genome at which more than one distinct allele is present.

The term ‘homozygous’, as used herein, identifies a locus in the genome at which no more than one distinct allele is present.

‘THCA’, as used herein, is defined as an abbreviation for tetrahydrocannabinolic acid. The present disclosure provides plants that are resistant to powdery mildew while also having high levels of THCA. THCA is the most abundant non-psychoactive cannabinoid found in cannabis, and is a precursor to tetrahydrocannabinol (THC), an active component of cannabis. Cannabinoids are compounds that can interact with the vertebrate endocannabinoid system by binding to cannabinoid receptors. Other cannabinoids include but are not limited to tetrahydrocannabinol (THC), cannabidiol (CBD), and cannabinol (CBN). THCA, as well as other cannabinoids and terpenes, is produced and stored in stalked glandular trichomes, specialized cell types that protrude from all leaf surfaces of the flower structure. The flowers of the Cannabis sativa L. plant are covered in these glandular trichomes. Certain leaves, colloquially referred to as ‘sugar leaves’, also display glandular trichomes, though at much lower density than is found on the flowers. In some embodiments, the isolated Cannabis sativa L. plant produces flowers with greater than 22% tetrahydrocannabinolic acid (THCA) (wt/wt). In some embodiments, the isolated plant produces flowers with greater than 23%, 24%, or 25% THCA (wt/wt).

Cannabinoid levels can be quantified with High Performance Liquid Chromatography—Diode-Array Detection (HPLC-DAD). A flower is ground and suspended in Methanol (HPLC-grade). The sample is vortexed for 15 minutes at maximum speed, then incubated in methanol at room temperature for 1 hour. The supernatant is filtered through a 0.22 μm PTFE filter, then diluted 20-fold in methanol before injection on the HPLC. The liquid sample is pressurized and passed through a column filled with solid adsorbent material. Different chemicals within the sample will adhere to the column at different strengths, resulting in these different chemicals reaching the end of the column after different amounts of time. By analyzing the wavelength of the separated chemicals as they are eluted off of the column, using an array of diodes, the chemical composition of a solution can be determined and quantification of a particular chemical performed against known reference standards and a calibration curve.

‘Harvestable material’, as used herein, includes flowers and/or flower buds, leaves, stalks, seeds, oils, and roots of a plant. ‘Harvestable floral material’, as used herein, refers to flowers and/or flower buds. In some embodiments, the plant produces at least 41 g per plant of harvestable floral material. In some embodiments, the plant produces at least 42 g, 43 g, 44 g, 45 g, 46 g, or 47 g per plant of harvestable floral material. In some embodiments, the harvestable floral material comprises flowers and/or flower buds. In some embodiments, the plant produces harvestable material comprising flowers and/or flower buds, leaves, stalks, and seeds.

A plant ‘part’, ‘tissue’, or ‘cell’, as used herein, refers to any portion of the vegetative or reproductive parts of the plant, including both roots and aerial portions of the plant, including but not limited to, seed (including mature seed and immature seed); a plant cutting; a plant cell; a plant cell culture; or a plant organ (e.g., pollen, embryos, flowers, fruits, shoots, trichomes, buds, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. Regenerable cells in a plant cell or tissue culture may be embryos, protoplasts, meristematic cells, callus, pollen, leaves, anthers, roots, root tips, silk, flowers, kernels, ears, cobs, husks, or stalks. In contrast, some plant cells are not capable of being regenerated to produce plants and are referred to herein as “non-regenerable” plant cells. In some embodiments, a plant part, tissue, or cell of the isolated Cannabis sativa L. plant is claimed. In some embodiments, the plant cell is a non-regenerable cell. In some embodiments, the isolated plant part further comprises harvestable material comprising flowers and/or flower buds, leaves, stalks, and seeds.

‘Lateral branching’, as used herein, refers to the phenomenon in which lateral buds off of the main stem of a plant produce outgrowths from the main stem. These lateral buds each have their own apical meristem and can produce their own flowers. ‘Increased lateral branching’ as used herein indicates that a plant or population of plants has at least 5%, at least 10%, at least 15%, at least 20%, at least 25%, at least 30%, at least 35%, at least 40%, at least 45%, at least 50% more lateral branches when compared to another plant or plant population.

The number and quality of flowers produced by a Cannabis sativa L. plant will vary in response to horticultural cultivation practices, such as the pruning strategy, defoliation schedule, and time spent in the vegetative growth phase. ‘Top of the canopy flowers’, as used herein, refer to flowers deriving from the terminal bud, the flowering site on a female cannabis plant where flowers grow together tightly. This is also known as a ‘cola’. Healthy plants typically form one main cola from the center of their structure and smaller colas on the outside of the plant. Top of the canopy flowers generally display increased trichome density, are richer in cannabinoids and terpenes, are denser, and have increased size when compared to flowers that develop from lateral buds. ‘Increased top of the canopy flowers’, as used herein, indicates that a plant or population of plants has at least 5%, at least 10%, at least 15%, at least 20%, at least 25%, at least 30%, at least 35%, at least 40%, at least 45%, at least 50% more ‘top of the canopy flowers’ when compared to another plant or plant population.

A ‘node’, as used herein, refers to a site on a plant stem from which lateral buds and leaves originate. An ‘internode’, as used herein, refers to the portion of a plant stem between one node and the adjacent node on the stem.

‘Flower site stacking’, as used herein, refers to the shortening of internode length on a plant, resulting in increased number of nodes, increased number of flowers, and increased yield. ‘Increased flower site stacking’, as used herein, indicates a plant or population of plants that has at least 5%, at least 10%, at least 15%, at least 20%, at least 25%, at least 30%, at least 35%, at least 40%, at least 45%, at least 50% more nodes when compared to another plant or plant population.

A ‘control plant’, as used herein, refers to reference plant to which an isolated plant is compared. In some embodiments, the control plant is a naturally-occurring Cannabis sativa L. plant. In some embodiments, the control plant is a naturally-occurring powdery mildew-resistant Cannabis sativa L. plant. In some embodiments, the control plant is from the ‘PNW39’ population of plants.

‘Crossing’, as used herein, refers to obtaining pollen or feminized pollen from a male or female plant, respectively, then manually providing that pollen to the flowers of a female plant in order for sexual reproduction to occur. In some embodiments, the method includes genotyping the offspring population of Cannabis sativa L. plants for the presence of one or more resistance genes. Several embodiments relate to a method of selecting a Cannabis sativa L. plant that is resistant to powdery mildew, comprising crossing a first Cannabis sativa L. plant resistant to powdery mildew to a second Cannabis sativa L. plant to produce a population of offspring Cannabis sativa L. plants; genotyping the offspring population of Cannabis sativa L. plants for the presence of one or more powdery mildew resistance genes; and selecting an offspring Cannabis sativa L. plant on the basis of the genotyping, wherein the selected offspring Cannabis sativa L. plant is resistant to powdery mildew. In some embodiments, the selected offspring Cannabis sativa L. plant produces flowers with greater than 22% tetrahydrocannabinolic acid (THCA) (wt/wt). In some embodiments, the selected offspring Cannabis sativa L. plant produces flowers with greater than 23%, 24%, or 25% THCA (wt/wt). In some embodiments, the resistance gene is located on Chromosome 2 of GenBank assembly accession no. GCA_900626175.2. In some embodiments, the resistance gene encodes a protein in the NB-ARC-LRR family. In some embodiments, the resistance gene encodes a transcript comprising a polynucleotide at least 75% identical, 80% identical, 85% identical, 90% identical, 95% identical, 96% identical, 97% identical, 98% identical, or 99% identical to a polynucleotide selected from the group consisting of SEQ ID NOs: 1-28. In some embodiments, the resistance gene encodes a transcript comprising a polynucleotide selected from the group consisting of SEQ ID NOs: 1-28. In some embodiments, the resistance gene encodes a transcript comprising a polynucleotide at least 75% identical, 80% identical, 85% identical, 90% identical, 95% identical, 96% identical, 97% identical, 98% identical, or 99% identical to a polynucleotide selected from the group consisting of SEQ ID NO: 6 and SEQ ID NO: 27. In some embodiments, the resistance gene encodes a transcript comprising a polynucleotide selected from the group consisting of SEQ ID NO: 6 and SEQ ID NO: 27. In some embodiments, the resistance gene encodes a transcript comprising SEQ ID NO: 6. In some embodiments, the resistance gene encodes a transcript comprising SEQ ID NO: 27. In some embodiments, the resistance gene encodes a protein comprising a polypeptide that is at least 70%, at least 75%, at least 80%, at least 85%, at least 90%, at least 95%, at least 96%, at least 97%, at least 98%, at least 99%, or 100% identical to a polypeptide selected from SEQ ID NOs: 29-56. In some embodiments, the resistance gene encodes a protein comprising a polypeptide selected from the group consisting of SEQ ID NOs: 29-56. In some embodiments, the resistance gene encodes a protein comprising a polypeptide at least 75% identical, 80% identical, 85% identical, 90% identical, 95% identical, 96% identical, 97% identical, 98% identical, or 99% identical to a polypeptide selected from the group consisting of SEQ ID NO: 34 and SEQ ID NO: 55. In some embodiments, the resistance gene encodes a protein comprising a polypeptide selected from the group consisting of SEQ ID NO: 34 and SEQ ID NO: 55. In some embodiments, the resistance gene encodes a protein comprising a polypeptide of SEQ ID NO: 34. In some embodiments, the resistance gene encodes a protein comprising a polypeptide of SEQ ID NO: 55. In some embodiments, the second Cannabis sativa L. plant is not resistant to powdery mildew. In some embodiments, the selected offspring Cannabis sativa L. plant produces at least 41 g per plant of harvestable floral material. In some embodiments, the selected offspring Cannabis sativa L. plant produces at least 42 g, 43 g, 44 g, 45 g, 46 g, or 47 g per plant of harvestable floral material. In some embodiments, the selected offspring Cannabis sativa L. plant has increased lateral branching as compared to the first Cannabis sativa L. plant. In some embodiments, the selected offspring Cannabis sativa L. plant has at least 5%, at least 10%, at least 15%, at least 20%, at least 25%, at least 30%, at least 35%, at least 40%, at least 45%, or at least 50% more top of the canopy flowers as compared to the first Cannabis sativa L. plant. In some embodiments, the selected offspring Cannabis sativa L. plant has increased flower site stacking as compared to the first Cannabis sativa L. plant.

‘Genotyping’, as used herein, refers to process of determining which allele or alleles is/are present in the genome of the plant at one or more given loci. Genotyping involves extracting a nucleic acid such as DNA or RNA, then performing common laboratory procedures to identify the present alleles, including but not limited to such techniques as polymerase chain reaction (PCR), restriction enzyme digest assays, restriction fragment length polymorphism identification (RFLPI), random amplified polymorphic detection (RAPD), amplified fragment length polymorphism detected (AFLPD), allele specific oligonucleotide (ASO) probes, microarrays, hybridization techniques, single nucleotide polymorphism (SNP) genotyping chips, Sanger sequencing, and next-generation sequencing. In some embodiments, provided herein are methods of identifying a Cannabis sativa L. plant that is resistant to powdery mildew, the method comprising genotype the plant for the presence of a powdery mildew resistance gene, wherein the powdery mildew resistance gene encodes a transcript comprising a polynucleotide selected from the group consisting of SEQ ID NOs: 1-28, and/or encodes a polypeptide that is at least 70%, at least 75%, at least 80%, at least 85%, at least 90%, at least 95%, at least 96%, at least 97%, at least 98%, at least 99%, or 100% identical to a polypeptide selected from SEQ ID NOs: 29-56. In some embodiments, the resistance gene encodes a transcript comprising a polynucleotide at least 75% identical, 80% identical, 85% identical, 90% identical, 95% identical, 96% identical, 97% identical, 98% identical, or 99% identical to a polynucleotide selected from the group consisting of SEQ ID NOs: 1-28. In some embodiments, the resistance gene encodes a transcript comprising a polynucleotide selected from the group consisting of SEQ ID NOs: 1-28. In some embodiments, the resistance gene encodes a transcript comprising a polynucleotide at least 75% identical, 80% identical, 85% identical, 90% identical, 95% identical, 96% identical, 97% identical, 98% identical, or 99% identical to a polynucleotide selected from the group consisting of SEQ ID NO: 6 and SEQ ID NO: 27. In some embodiments, the resistance gene encodes a transcript comprising a polynucleotide selected from the group consisting of SEQ ID NO: 6 and SEQ ID NO: 27. In some embodiments, the resistance gene encodes a transcript comprising SEQ ID NO: 6. In some embodiments, the resistance gene encodes a transcript comprising SEQ ID NO: 27. In some embodiments, the resistance gene encodes a protein comprising a polypeptide that is at least 70%, at least 75%, at least 80%, at least 85%, at least 90%, at least 95%, at least 96%, at least 97%, at least 98%, at least 99%, or 100% identical to a polypeptide selected from SEQ ID NOs: 29-56. In some embodiments, the resistance gene encodes a protein comprising a polypeptide selected from the group consisting of SEQ ID NOs: 29-56. In some embodiments, the resistance gene encodes a protein comprising a polypeptide at least 75% identical, 80% identical, 85% identical, 90% identical, 95% identical, 96% identical, 97% identical, 98% identical, or 99% identical to a polypeptide selected from the group consisting of SEQ ID NO: 34 and SEQ ID NO:55. In some embodiments, the resistance gene encodes a protein comprising a polypeptide selected from the group consisting of SEQ ID NO: 34 and SEQ ID NO: 55. In some embodiments, the resistance gene encodes a protein comprising a polypeptide of SEQ ID NO: 34. In some embodiments, the resistance gene encodes a protein comprising a polypeptide of SEQ ID NO: 55.

Unless otherwise stated, nucleic acid sequences in the text of this specification are provided in the 5′ to 3′ direction when read from left to right. One of skill in the art would be aware that a given DNA sequence is understood to define a corresponding RNA sequence which is identical to the DNA sequence except for replacement of the thymine (T) nucleotides of the DNA with uracil (U) nucleotides. Thus, providing a specific DNA sequence is understood to define the RNA equivalent and vice versa. A given first polynucleotide sequence, whether DNA or RNA, further defines the sequence of its exact complement (which can be DNA or RNA), i.e., a second polynucleotide that hybridizes perfectly to the first polynucleotide by forming Watson-Crick base-pairs.

The term ‘polynucleotide’, as used herein, commonly refers to a DNA or RNA molecule containing multiple nucleotides and generally refers both to ‘oligonucleotides’ (a polynucleotide molecule of 18-25 nucleotides in length) and longer polynucleotides of 26 or more nucleotides.

The term ‘polypeptide’, as used herein, commonly refers to a protein molecule containing multiple amino acids.

‘Percent (%) identity’ of a sequence, as used herein with respect to nucleotide or protein sequences, is defined as the percentage of nucleotides or amino acid residues in a candidate sequence that are identical or homologous with the nucleotides or amino acid residues in the polynucleotide or polypeptide being compared, after aligning the sequences considering any conservative substitutions as part of the sequence identity. Homology between polynucleotides is determined based on a substitution matrix, such as is described in: Smith and Waterman (1981); Needleman and Wunsch (1970); Pearson and Lipman (1988); Higgins and Sharp (1988); Higgins and Sharp (1989); Corpet et al., (1988); Huang et al., (1992); and Pearson et al., (1994). Altschul et al., (1994) presents a detailed consideration of sequence alignment methods and homology calculations. Homology between different amino acid residues is determined based on a substitution matrix, such as the BLOSUM (BLOcks SUbstitution Matrix alignment). Determining percent sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

The NCB1 Basic Local Alignment Search Tool [(BLAST) (Altschul et al., 1990) is available from several sources, including the National Center for Biotechnology Information (NCB1, Bethesda, MD) and on the Internet, for use in connection with the sequence analysis programs blastp, blastn, blastx, tblastn and tblastx.

In one embodiment, the plant is resistant to infection by fungi of the family Erysiphaceae, including genera Arthrocladiella, Blumeria, Brasiliomyces Bulbomicrosphaera, Bulbouncinula, Caespitotheca, Cystotheca, Erysiphe (Oidium), Golovinomyces, Leveillula (Oidiopsis), Medusosphaera, Microsphaera, Neoerysiphe, Oidium, Phyllactinia (Ovulariopsis), Pleochaeta, Podosphaera, Sawadaea, Setoerysiphe, Sphaerotheca, Typhulochaeta, Uncinula, and Uncinuliella.

In one embodiment, the Cannabis sativa L. plant is resistant to infection by fungi of the genus Golovinomyces. In some embodiments, the isolated plant is resistant to infection by fungi of the species G. adenophorae, G. ambrosiae, G. americanus, G. andinus, G. arabidis, G. artemisiae, G. asperifolii, G. asperifoliorum, G. asterum, G. biocellatus, G. bolayi, G. brunneopunctatus, G. calceolariae, G. californicus, G. caulicola, G. chrysanthemi, G. cichoracearum, G. clematidis, G. cucurbitacearum, G. cynoglossi, G. depressus, G. echinopis, G. euphorbiicola, G. fischeri, G. franseriae, G. glandulariae, G. greeneanus, G. hydrophyllacearum, G. hyoscyami, G. immersus, G. inulae, G. laporteae, G. latisporus, G. longipes, G. lycopersici, G. macrocarpus, G. magnicellulatus, G. monardae, G. montagnei, G. neosalviae, G. ocimi, G. orontii, G. poonaensis, G. prenanthis, G. pseudosepultus, G. riedlianus, G. robustus, G. rogersonii, G. rubiae, G. salvia, G. senecionis, G. simplex, G. sonchicola, G. sordidus, G. spadiceus, G. sparsus, G. tabaci, G. valerianae, G. verbasci, G. verbenae, and G. vincae. In one embodiment, the plant is resistant to infection by fungi of the species Golovinomyces ambrosiae.

In some embodiments, the plant has one or more powdery mildew resistance genes. In some embodiments, the plant has one powdery mildew resistance gene. In some embodiments, the plant has more than one powdery mildew resistance gene. In some embodiments, the plant has two, three, or four powdery mildew resistance genes. In some embodiments, the one or more powdery mildew resistance gene is located on Chromosome 2 of GenBank assembly accession no. GCA_900626175.2. In some embodiments, the resistance gene encodes a protein in the NB-ARC-LRR family. In some embodiments, the resistance gene encodes a transcript comprising a polynucleotide at least 75% identical, 80% identical, 85% identical, 90% identical, 95% identical, 96% identical, 97% identical, 98% identical, or 99% identical to a polynucleotide selected from the group consisting of SEQ ID NOs: 1-28. In some embodiments, the one or more resistance gene encodes one or more transcripts comprising a polynucleotide selected from the group consisting of SEQ ID NOS: 1-28. In some embodiments, the resistance gene encodes a transcript comprising a polynucleotide at least 75% identical, 80% identical, 85% identical, 90% identical, 95% identical, 96% identical, 97% identical, 98% identical, or 99% identical to a polynucleotide selected from the group consisting of SEQ ID NO: 6 and SEQ ID NO:27. In some embodiments, the resistance gene encodes a transcript comprising SEQ ID NO: 6. In some embodiments, the resistance gene encodes a transcript comprising SEQ ID NO: 27. In some embodiments, the one or more resistance genes encodes one or more proteins comprising at least one polypeptide that is at least 70%, at least 75%, at least 80%, at least 85%, at least 90%, at least 95%, at least 96%, at least 97%, at least 98%, at least 99%, or 100% identical to a polypeptide selected from the group consisting of SEQ ID NOs: 29-56. In some embodiments, the resistance gene encodes a protein comprising a polypeptide selected from the group consisting of SEQ ID NOs: 29-56. In some embodiments, the resistance gene encodes a protein comprising a polypeptide at least 75% identical, 80% identical, 85% identical, 90% identical, 95% identical, 96% identical, 97% identical, 98% identical, or 99% identical to a polypeptide selected from the group consisting of SEQ ID NO: 34 and SEQ ID NO: 55. In some embodiments, the resistance gene encodes a protein comprising a polypeptide selected from the group consisting of SEQ ID NOs: 34 and 55. In some embodiments, the resistance gene encodes a protein comprising a polypeptide of SEQ ID NO: 34. In some embodiments, the resistance gene encodes a protein comprising a polypeptide of SEQ ID NO: 55. In some embodiments, the plant comprises more than one resistance gene. In some embodiments, the plant comprises two, three, or four resistance genes. In some embodiments, the resistance gene allele is a dominant allele. In some embodiments, the plant is homozygous for the resistance gene allele. In some embodiments, the plant is heterozygous for the resistance gene allele.

In some embodiments, the powdery mildew resistance gene encodes an NB-LRR family disease resistance protein. In some embodiments, the powdery mildew resistance gene encodes a disease resistance protein in InterPro family IPR044974. In some embodiments, the one or more powdery mildew resistance genes encodes a protein comprising a leucine-rich repeat domain (accession no. c134836 as defined by NCBI Conserved Domain Database; and/or IPR032675 as defined by InterProScan). In some embodiments, the one or more powdery mildew resistance genes encodes a protein comprising an NB-ARC domain (accession no. pfam00931 as defined by NCBI Conserved Domain Database; and/or IPR002182 as defined by InterProScan). In some embodiments, the one or more powdery mildew resistance genes encodes a protein comprising an RX-CC-like coiled-coil domain (accession no. cd14798 as defined by NCBI Conserved Domain Database; and/or IPR038005 as defined by InterProScan). In some embodiments, the one or more powdery mildew resistance genes encodes a protein comprising both an RX-CC-like coiled-coil domain and an NB-ARC domain. In some embodiments, the one or more powdery mildew resistance genes encodes a protein comprising both an RX-CC-like coiled-coil domain and a leucine-rich repeat domain. In some embodiments, the one or more powdery mildew resistance genes encodes a protein comprising both a leucine-rich repeat and an NB-ARC domain. In some embodiments, the one or more powdery mildew resistance genes encodes a protein comprising a leucine-rich repeat, an RX-CC-like coiled-coil domain, and an NB-ARC domain. In some embodiments, the one or more powdery mildew resistance genes encodes a protein comprising a PLN03150 domain (accession no. PLN03150 as defined by NCBI Conserved Domain Database). In some embodiments, the one or more powdery mildew resistance genes encodes a protein comprising a PLN00113 domain (accession no. PLN00113 as defined by NCBI Conserved Domain Database).

In some embodiments, the isolated powdery mildew resistant Cannabis sativa L. plant has flowers that contain more than 22%, 23%, 24%, 25%, or 26% THCA (wt/wt). In some embodiments, the isolated powdery mildew resistant Cannabis sativa L. plant has flowers that contain on average more than 22%, 23%, 24%, 25%, or 26% THCA (wt/wt).

In some embodiments, the invention provides an isolated plant resistant to powdery mildew which also has increased yield of harvestable floral material over a control Cannabis sativa L. plant. In some embodiments, the plant yields at least 41 g, 42 g, 43 g, 44 g, 45 g, 46 g, or 47 g of harvestable floral material per plant. In some embodiments, the plant provides harvestable floral material comprising flowers and/or flower buds. In one embodiment, the plant provides harvestable material comprising flowers and/or flower buds, leaves, stalks, seeds, oils, and roots.

In one embodiment, this invention provides a plant part, tissue, or cell from a powdery mildew resistant Cannabis sativa L. plant, including any portion of the vegetative or reproductive parts of the plant, including both roots and aerial portions of the plant, including but not limited to, a cell, protoplast, embryo, pollen grain, ovule, flower, leaf, stem, cotyledon, hypocotyl, meristematic cell, rootstock, root, root tip, pistil, anther, shoot tip, shoot, fruit, seed, and petiole.

In some embodiments, the isolated powdery mildew resistant Cannabis sativa L. plant has increased lateral branching over a naturally-occurring powdery mildew resistant Cannabis sativa L. plant. In some embodiments, the isolated powdery mildew resistant Cannabis sativa L. plant has at least 5%, at least 10%, at least 15%, at least 20%, at least 25%, at least 30%, at least 35%, at least 40%, at least 45%, at least 50% more lateral branches when compared to a control Cannabis sativa L. plant.

In some embodiments, the isolated powdery mildew resistant Cannabis sativa L. plant has an increased number of ‘top of the canopy flowers’ when compared to a control Cannabis sativa L. plant. In some embodiments, the isolated powdery mildew resistant Cannabis sativa L. plant has at least 5%, at least 10%, at least 15%, at least 20%, at least 25%, at least 30%, at least 35%, at least 40%, at least 45%, at least 50% more ‘top of the canopy flowers’ when compared to a control Cannabis sativa L. plant.

In some embodiments, the isolated powdery mildew resistant Cannabis sativa L. plant has increased flower site stacking when compared to a control Cannabis sativa L. plant. In some embodiments, the isolated powdery mildew resistant Cannabis sativa L. plant has at least 5%, at least 10%, at least 15%, at least 20%, at least 25%, at least 30%, at least 35%, at least 40%, at least 45%, at least 50% more nodes when compared to a control Cannabis sativa L. plant.

Certain embodiments of the present invention relate to methods for selecting a Cannabis sativa L. plant resistant to powdery mildew, comprising crossing a first Cannabis sativa L. plant resistant to powdery mildew to a second Cannabis sativa L. plant to produce a population of offspring Cannabis sativa L. plants; genotyping the offspring population of Cannabis sativa L. plants for the presence of one or more powdery mildew resistance genes; and selecting an offspring Cannabis sativa L. plant that is resistant to powdery mildew. In some embodiments, the second Cannabis sativa L. plant is not resistant to powdery mildew.

In some embodiments, the method includes selecting an offspring Cannabis sativa L. plant based on the results of genotyping, such that the selected plant will be resistant to powdery mildew. In some embodiments, the selected offspring Cannabis sativa L. plant produces flowers with greater than 22% tetrahydrocannabinolic acid (THCA) (wt/wt). In some embodiments, the selected offspring Cannabis sativa L. plant produces flowers with greater than 23%, 24%, or 25% THCA (wt/wt). In some embodiments, the resistance gene is located on Chromosome 2 of GenBank assembly accession no. GCA_900626175.2. In some embodiments, the resistance gene encodes a protein in the NB-ARC-LRR family. In some embodiments, the resistance gene encodes a transcript comprising a polynucleotide at least 75% identical, 80% identical, 85% identical, 90% identical, 95% identical, 96% identical, 97% identical, 98% identical, or 99% identical to a polynucleotide selected from the group consisting of SEQ ID NOs: 1-28. In some embodiments, the sequence of the resistance gene encodes one or more transcripts comprising a polynucleotide selected from the group consisting of SEQ ID NOs: 1-28. In some embodiments, the resistance gene encodes a transcript comprising a polynucleotide at least 75% identical, 80% identical, 85% identical, 90% identical, 95% identical, 96% identical, 97% identical, 98% identical, or 99% identical to a polynucleotide selected from the group consisting of SEQ ID NO: 6 and SEQ ID NO: 27. In some embodiments, the resistance gene encodes a transcript comprising a polynucleotide selected from the group consisting of SEQ ID NO: 6 and SEQ ID NO: 27. In some embodiments, the resistance gene encodes a transcript comprising SEQ ID NO: 6. In some embodiments, the resistance gene encodes a transcript comprising SEQ ID NO: 27. In some embodiments, the powdery mildew resistance gene encodes a protein comprising one or more polypeptides selected from the group consisting of SEQ ID NOs: 29-56. In some embodiments, the one or more powdery mildew resistance genes encodes a protein comprising a sequence that is at least 70%, at least 75%, at least 80%, at least 85%, at least 90%, at least 95%, at least 96%, at least 97%, at least 98%, at least 99%, or 100% identical to one or more of SEQ ID NOs: 29-56. In some embodiments, the resistance gene encodes a protein comprising a polypeptide selected from the group consisting of SEQ ID NOs: 29-56. In some embodiments, the resistance gene encodes a protein comprising a polypeptide at least 75% identical, 80% identical, 85% identical, 90% identical, 95% identical, 96% identical, 97% identical, 98% identical, or 99% identical to a polypeptide selected from the group consisting of SEQ ID NO: 34 and SEQ ID NO:55. In some embodiments, the resistance gene encodes a protein comprising a polypeptide selected from the group consisting of SEQ ID NO: 34 and SEQ ID NO: 55. In some embodiments, the resistance gene encodes a protein comprising a polypeptide of SEQ ID NO: 34. In some embodiments, the resistance gene encodes a protein comprising a polypeptide of SEQ ID NO: 55. In some embodiments, the one or more powdery mildew resistance genes encodes an NB-LRR family disease resistance protein. In some embodiments, the one or more powdery mildew resistance genes encodes a protein comprising a leucine-rich repeat (accession no. c134836 as defined by NCBI Conserved Domain Database; and/or IPR032675 as defined by InterProScan), an NB-ARC domain (accession no. pfam00931 as defined by NCBI Conserved Domain Database; and/or IPR002182 as defined by InterProScan), and/or an RX-CC-like coiled-coil domain (accession no. cd14798 as defined by NCBI Conserved Domain Database; and/or IPR038005 as defined by InterProScan). In some embodiments, the one or more powdery mildew resistance genes encodes a protein comprising a PLN03150 domain (accession no. PLN03150 as defined by NCBI Conserved Domain Database). In some embodiments, the selected offspring Cannabis sativa L. plant produces at least 41 g per plant of harvestable floral material. In some embodiments, the selected offspring Cannabis sativa L. plant produces at least 42 g, 43 g, 44 g, 45 g, 46 g, or 47 g per plant of harvestable floral material. In some embodiments, the selected offspring Cannabis sativa L. plant has increased lateral branching as compared to the first Cannabis sativa L. plant. In some embodiments, the selected offspring Cannabis sativa L. plant has at least 5%, at least 10%, at least 15%, at least 20%, at least 25%, at least 30%, at least 35%, at least 40%, at least 45%, or at least 50% more top of the canopy flowers as compared to the first Cannabis sativa L. plant. In some embodiments, the selected offspring Cannabis sativa L. plant has increased flower site stacking as compared to the first Cannabis sativa L. plant.

### EXAMPLES

The presently disclosed subject matter will be better understood by reference to the following Examples, which are provided as exemplary of the invention, and not by way of limitation.

### Example 1: Identification of a Powdery Mildew Resistant Plant

This Example demonstrates that resistance to powdery mildew is possible in Cannabis sativa L.

Resistance to powdery mildew was first observed in photoperiod-sensitive experimental line “PNW39” after being grown in a greenhouse setting with high disease pressure. Two independent mapping populations were subsequently developed using parental material consisting of “PNW39,” susceptible and photoperiod-sensitive cultivar “Jumping Jack,” and a susceptible and photoperiod-insensitive recombinant inbred family “Dwy1337.” For the first population, feminized pollen from susceptible cultivar “Jumping Jack” was crossed to the female parent “PNW39.” The second population was developed by exposing female parent “PNW39” to an open pollination of multiple individuals from the “Dwy1337” family. At least 100 viable seeds were collected from each independent cross. Currently, the most prevalent accessions of C. sativa are highly heterozygous due to natural preference of the plant for outcrossing and acute inbreeding depression (Onofri and Mandolino (2017) Cannabis sativa L. Botany and Biotechnology, eds S. Chandra, H. Lata, and M. A. ElSohly (Cham: Springer): 319-342). Thus, the F1 families resulting from these hybridizations were genetically heterogeneous and allowed for linkage mapping of simple to moderately complex traits.

Identification of the disease as powdery mildew as caused by an infection of Golovinomyces ambrosiae was carried out according to known methods in the field.

### Example 2: a Single Dominant Gene is Likely Associated with Disease Resistance

This Example demonstrates that powdery mildew resistance is controlled by a single, dominant locus.

Eighty-nine F1 progeny from “PNW39”×“Jumping Jack” (Trial 1) and 96 F1 progeny from “PNW39”×“Dwy1337” (Trial 2) were grown within high-humidity (60-80%) enclosures under high pressure sodium light bulbs with daytime high temperatures of 25-28 °C and nighttime low temperatures of 21-24 °C. Infected plants serving as powdery mildew inoculants surrounded experimental plants for the duration of each trial. White colonies developed on leaves and stems of both mapping populations within 45 days of seedling germination. Plants were maintained through maturity (flowering) to confirm the integrity of resistance in all phenological stages. Plants were scored as “resistant” or “susceptible” since intermediate phenotypes were not apparent. Fifty-five clonally propagated individuals from the “PNW39”×“Jumping Jack” population were re-infected to provide replication and validation of initial scores. Thus, segregating phenotypic resistance was observed in three separate trials spanning two genetic backgrounds.

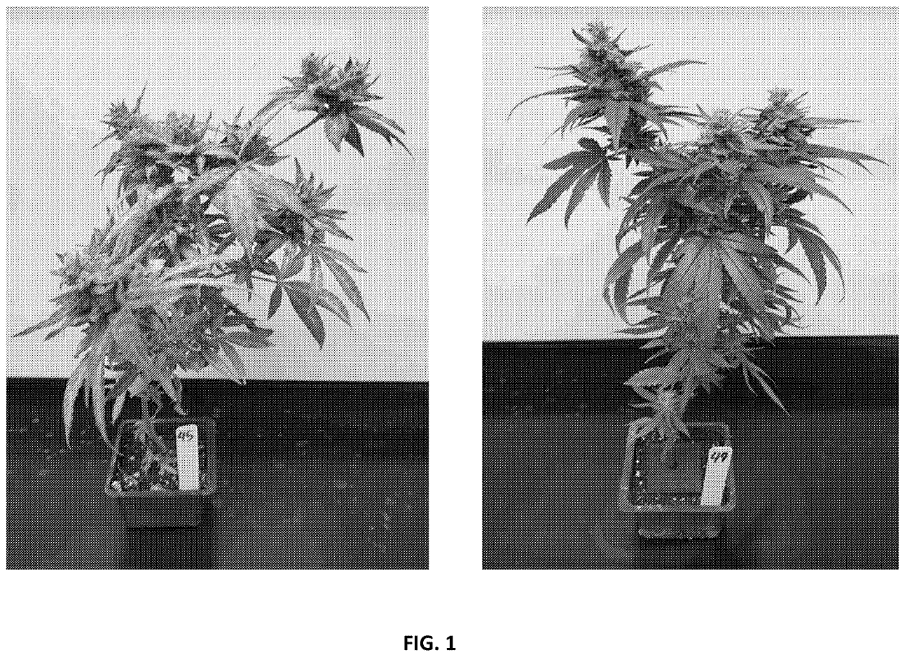

Dense, powdery, white mycelial growth developed on susceptible individuals (FIG. 1, left) whereas resistant plants remained free of any visible signs of infection (FIG. 1, right). Fungal colonies were not detected on “PNW39,” acting as a resistant control during each trial. Phenotyping the replicated subset of “PNW39”×“Jumping Jack” validated the results from the first trial.

A chi-squared test was conducted to evaluate progeny segregation against the null hypothesis of a single dominant Mendelian gene inherited from a heterozygous resistant parent. Both populations fit this assumption (Table 1), providing evidence that not only is there a single candidate locus, but its efficacy and heritability holds up in multiple genetic backgrounds.

TABLE 1
Chi-squared tests for goodness of fit among each mapping population.
To satisfy the null hypothesis, a 1:1 segregation is expected
of resistant (R) to susceptible (S) individuals.

| Female | | | | | | | | |
|---|---|---|---|---|---|---|---|---|
| (R) | Male (S) | R Progeny | | S Progeny | | Test for 1:1 Segregation | | |
| Parent | Parent | Obs. | Exp. | Obs. | Exp. | χ² | df | P |
| “PNW39” | “Jumping | 39 | 44.5 | 50 | 44.5 | 1.3596 | 1 | 0.2436 |
| | Jack” | | | | | | | |
| “PNW39” | “Dwy1337” | 49 | 48 | 47 | 48 | 0.0417 | 1 | 0.8383 |

### Example 3: The Powdery Mildew Resistance Gene Co-Localizes with SNP Marker LH3804 on Chromosome 2

This Example demonstrates that the powdery mildew resistance locus is located on the tail end of Chromosome 2.

DNA was extracted from parents and progeny of the “PNW39”×“Jumping Jack” population using a Quick-DNA Plant/Seed Miniprep Kit (Zymo Research) following the manufacturer's instructions. High-density genome-wide markers were generated for all 91 samples (2 parents and 89 progeny) using an Illumina iSelect 40K SNP Array by Lighthouse Genomics (Vancouver, British Columbia, Canada). Sequencing was performed by Génome Québec (Montréal, Quebec, Canada). DNA from the “PNW39”×“Dwy1337” population was extracted by the same method and used for marker validation experiments.

A total of 39,801 SNP markers were curated for the “PNW39”×“Jumping Jack” population, expressed as genotypes “AA,” “AB,” and “BB.” For quality filtering, the following types of markers were removed: (i) those with >10% missing data, (ii) those with monomorphic allele scores, and (iii) markers with no “AB” genotypes, as all polymorphic loci in this population structure should have heterozygous clusters.

The remaining markers were divided into three categories (B3.7, D1.10, D2.15) based on the notation described by Wu et al. ((2002) Theor. Popul. Biol. 61:349-363). Markers were characterized as type B3.7 (ab×ab) if both parental genotypes were heterozygous, then saved for linkage map construction if the progeny segregated in the expected ratio of 1:2:1 (aa:ab:bb) according to a chi-squared test. This resulted in a final total of 1,889 high-quality type B3.7 SNP markers. The first stage of genetic mapping in F1 populations includes generating separate linkage maps for each heterozygous parent; thus, the remaining markers were characterized as type D1.10 (ab×aa) or D2.15 (aa×ab). Markers were discarded if the progeny failed to reflect 1:1 segregation according to a chi-squared test. This resulted in a total of 3,779 type D1.10 markers and 4,220 type D2.15 markers.

Linkage maps were created in R (R Core Team, 2018) using the BatchMap (Schiffthaler et al. (2017) PLoS ONE 12:e0189256) package and its dependency OneMap (Margarido et al. (2007) Hereditas 144:78-79), which are particularly useful for linkage map construction in outcrossing F1 populations, with the former using multi-threaded computing for memory intensive phasing and marker ordering algorithms. The last filtering step utilized the function create.data.bins to minimize computing requirements by assigning only one representative bin to groups of redundant markers (those which exhibited zero recombination events between them). This resulted in a total of 2,051 unique high-quality SNP markers suitable for linkage map construction: 621 and 670 characterized as types D1.10 and D2.15, respectively, with 760 type B3.7 markers used to bridge the two linkage maps. Finally, a “dummy marker,” representing the binary powdery mildew responses, was included to elucidate its genetic linkage to the known SNPs. Linkage groups were established with a minimum Logarithm of the Odds (LOD) threshold of 10 and maximum recombination frequency of 0.35.

A total of 2,051 SNP markers were anchored to 28 linkage groups (14 for each parent), some with as few as 8 markers. Since the C. sativa genome only has 10 pairs of chromosomes, the smaller linkage groups are likely artifacts of chromosomal regions with unconfirmed linkage to larger fragments. The powdery mildew resistant trait associated with a 141-marker linkage group, which was identified as part of chromosome 2 according to the “CBDRx” genome assembly (accession no. LR213632.1).

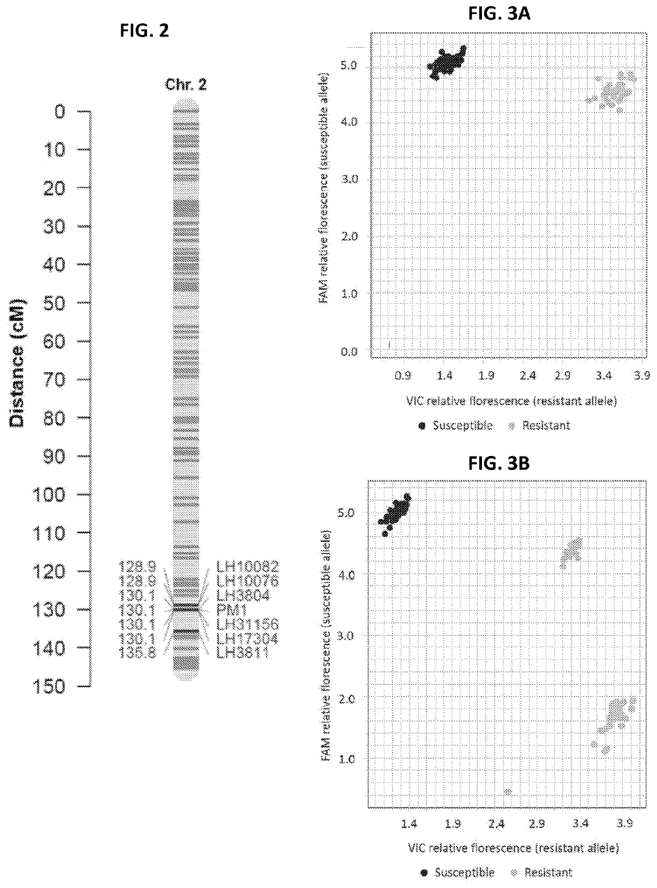

Binary disease scores co-segregated with one type D1.10 SNP marker, locus LH3804, in 100% of progeny. The probe sequence for this locus was provided by Lighthouse Genomics and used to search publicly available whole genome sequences from C. sativa to identify the physical location of the SNP. Publicly available genomes were queried using the MegaBLAST tool in the NCBI whole-genome shotgun contigs database; the genome for publicly available C. sativa cultivar “CBDRx” (GenBank assembly accession no. GCA_900626175.2) was downloaded and searched using the same tool implemented in Geneious Prime. The location of this marker bin (130.1/145.3 cM, or 95.39/96.15 Mb) placed locus LH3804 in a distal position on the long arm of chromosome 2 of “CBDRx” (accession no. LR213632.1, formerly chromosome 6, accession no. UZAU01000623.1) (FIG. 2). The physical locations of the probe sequences for the additional markers on the linkage map largely corroborate their placement on “CBDRx” chromosome 2, except for proximal regions with low marker density.

Primers Mildewey_1F (5′ TGAGGATGATCCAATGCCAACA 3′, SEQ ID NO: 57) and Mildewey_1R (5′ ACGACAATGTTCATGAGACAACA 3′, SEQ ID NO: 58) were then designed to amplify a 641 bp region surrounding the probe sequence. PCRs were carried out for the parents “PNW39” and “Jumping Jack” and selected progeny from both segregating populations in 20 μL reactions containing: 10 μL 2× Platinum™ II Hot-Start PCR Master Mix (ThermoFisher), 0.8 μL of 5 μM each primer (Integrated DNA Technologies), molecular grade water, and 2 μL template DNA (variable concentrations). Cycling conditions used were initial denaturing at 94 °C for 2 minutes followed by 35 cycles of 98 °C for 5 seconds and 60 °C for 15 seconds. PCR reactions were visualized as described in Example 1; all products were of the expected size. PCR cleanup, sequencing, and editing of sequence data were also as in Example 1.

Sanger sequencing confirmed the presence of an A/G mutation in accordance with the SNP array at locus LH3804. Flanking region sequences were used to design a TaqMan® SNP Genotyping Assay (ThermoFisher), which employed two primers (5′ TCATCCATCTATCTTGTGTTATTTCATTGCT 3′, SEQ ID NO: 59; and 5′ ACACTATCCTCTGATTCTGCATTCA 3′, SEQ ID NO: 60) and two probes (5′/VIC/TGCATTTTCTGTGTTGGCAT/MGB-NFQ/3′, SEQ ID NO: 61; and 5′/FAM/TGCATTTTCTGTATTGGCAT/MGB-NFQ 3′, SEQ ID NO: 62). This SNP assay was used to test progeny from both segregating populations in 10 μL qPCR reactions containing: 5 μL TaqPath™ ProAmp™ Master Mix (Applied Biosystems), 0.5 μL of the SNP Genotyping Assay (20×), 1 μL of template DNA, and water. Samples were run in a QuantStudio™ 5 (Applied Biosystems) using the “Fast” and “Genotyping” protocol with the following cycling conditions: a pre-read at 60 °C for 30 seconds; 95 °C for 5 minutes; 40 cycles of 95 °C for 5 seconds followed by 60 °C for 30 seconds; and a post-read at 60 °C for 30 seconds. Genotyping calls were made automatically by the QuantStudio™ 5 software and manually checked for accuracy.

The qPCR SNP genotyping assay PM_LH3804 classified F1 progeny from the “PNW39”×“Jumping Jack” population in two distinct clusters representing the susceptible (AA) and resistant (AG) individuals (FIG. 3A). The SNP genotyping assay also confirmed that the resistant parent “PNW39” was heterozygous for the resistance allele. None of the individuals were classified as “GG,” in accordance with hypothesized inheritance patterns.

PM_LH3804 assay results from the “PNW39”×“Dwy1337” cross were unexpected insofar as a third genotype (representing “GG”) was indicated in the SNP analysis (FIG. 3B) despite homozygous resistance being unlikely in this population. All susceptible progeny were scored as “AA,” but two genotype clusters represented the resistant progeny (19 individuals with “AG” and 30 individuals with “GG”) (FIG. 3B). Despite the anomaly no recombinants were observable in this second population.

All susceptible progeny were scored in the same homozygous cluster, but the resistant progeny were split into two groups consisting of both the expected heterozygous cluster and an unexpected cluster for homozygous resistance. One plausible explanation is the presence of a third allele or a deletion event in the “Dwy1337” genome which would not be amplified in the qPCR assay designed for one of the two allele possibilities discovered by the SNP array. In this scenario, heterozygous individuals could erroneously be scored as homozygous for the resistance allele. While DNA from the original “Dwy1337” pollen donors are not available (and alone would not provide a sufficient answer), this hypothesis was explored further by screening several “Dwy1337” families derived from single-seed descent of original susceptible parental material. All individuals tested (n=32) were either homozygous susceptible or failed to amplify, further suggesting the presence of a third allele not being detected by the SNP genotyping assay. Sanger sequencing was performed on several of the progeny from this population using the Primers Mildewey_1F and Mildewey_1R, and several redesigns of these primers in close proximity, yet provided no clear indication of a third allele that would not be amplified by the PM_LH3804 assay. These results may support the deletion event theory, or perhaps indicate another sequence variant that cannot be amplified using the end-point primers that were tested. Despite this anomaly, PM_LH3804 accurately detected the resistance-associated allele in all tested progeny with no observable recombination events.

### Example 4: The Powdery Mildew Resistance Gene Lies within a Cluster of R Genes

This Example demonstrates that the linked SNP is associated with a cluster of resistance R genes, one of which is likely the causal gene for powdery mildew resistance.

Primers Mildewey_1F (5′ TGAGGATGATCCAATGCCAACA 3′, SEQ ID NO: 57) and Mildewey_1R (5′ ACGACAATGTTCATGAGACAACA 3′, SEQ ID NO: 58) were used to amplify and sequence a 641 bp fragment surrounding the LH3804 locus using DNA from the resistant parent “PNW39” (nucleotide sequence supplied as SEQ ID NO: XX). MegaBLAST and BLASTn searches of the NCBI database with the built consensus sequence confirmed the BLAST results of the probe alone as being located on chromosome 2 of CBDRx and further resulted in hits to several predicted, putative, and probable disease resistance proteins on the same chromosome. A wider search of the region was conducted of the annotated CBDRx genome and a cluster of 10 putative disease resistance proteins were identified in the area surrounding the LH3804 locus, of which all contained N-terminal coiled-coil (CC) and nucleotide binding arc (NB-ARC) domains; two genes also contained leucine-rich repeats characteristic of plant R genes (Table 2). Three additional annotations were observed for pentatricopeptide repeat-containing proteins (Table 2), which are not usually implicated directly in disease resistance, but are assumed to share similar evolutionary histories with R genes and are typically found in clusters in the vicinity of R genes (Hernandez Mora et al. (2010) BMC Plant Biol 10:35).

TABLE 2
Annotated resistance-like genes on chromosome 2 of the Cannabis sativa
“CBDRx” genome surrounding the location of the LH3804 locus.

| | | | Conserved | | |
|---|---|---|---|---|---|
| Accession | Location ID | Description | domains* | Start | End |
| XM030647777.1 | LOC115718950 | Putative disease | CC, NBS, LRR | 94,007,316 | 94,011,7 [symbol] |
| | | resistance protein | | | |
| | | At1g50180 | | | |
| XM030649676.1 | LOC115720533 | Probable disease | CC, NBS | 94,017,111 | 94,022,9 [symbol] |
| | | resistance protein | | | |
| | | At1g58602 | | | |
| XM030648606.1 | LOC115719526 | Putative disease | CC, NBS | 95,314,496 | 95,318,7 [symbol] |
| | | resistance protein | | | |
| | | At1g50180 | | | |
| XM030648609.1 | LOC115719528 | Pentatricopeptide repeat- | — | 95,318,784 | 95,322,7 [symbol] |
| | | containing protein-2C, | | | |
| | | transcript variant X3 | | | |
| XM030648607.1 | LOC115719528 | Pentatricopeptide repeat- | — | 95,318,784 | 95,322,7 [symbol] |
| | | containing protein-2C, | | | |
| | | transcript variant X1 | | | |
| XM030648608.1 | LOC115719528 | Pentatricopeptide repeat- | — | 95,318,784 | 95,322,7 [symbol] |
| | | containing protein-2C, | | | |
| | | transcript variant X2 | | | |
| XM030648562.1 | LOC115719488 | Probable disease | CC, NBS | 95,390,125 | 95,393,6 [symbol] |
| | | resistance protein RF9 | | | |
| XM030648563.1 | LOC115719489 | Putative disease | CC, NBS, LRR | 95,394,038 | 95,397,6 [symbol] |
| | | resistance protein | | | |
| | | At1g50180 | | | |
| XM030649700.1 | LOC115720557 | Putative disease | CC, NBS | 95,402,526 | 95,410,0 [symbol] |
| | | resistance protein | | | |
| | | At1g50180 | | | |
| XM030648564.1 | LOC115719490 | Putative disease | CC, NBS | 95,412,644 | 95,418,3 [symbol] |
| | | resistance protein | | | |
| | | At1g50180 | | | |
| XM030649701.1 | LOC115720558 | Putative disease | CC, NBS | 95,421,091 | 95,424,6 [symbol] |
| | | resistance protein | | | |
| | | At1g50180 | | | |
| XM030648577.1 | LOC115719504 | Putative disease | CC, NBS | 95,432,966 | 95,437,8 [symbol] |
| | | resistance protein | | | |
| | | At1g50180 | | | |
| XM030648571.1 | LOC115719497 | Probable disease | CC, NBS, LRR** | 95,496,187 | 95,505,6 [symbol] |
| | | resistance RPP8-like | | | |
| | | protein 2 | | | |

*Conserved domains based on searches in the NCBI conserved domains database; CC, N-terminal coiled coil domain (accession no. cd14798); NBS, nucleotide binding site domain (accession no. cl26397); LRR, leucine-rich repeat (accession no. cl34836). A dash (—) indicates that the sequence was not used to query the conserved domains database.
**Contains provisional leucine-rich repeat receptor-like protein kinase (PLN00113 super family, accession no. cl33414). [symbol]
indicates data missing or illegible when filed

### Example 5: Hybrid Cultivars are Both Resistant to Powdery Mildew and have Significantly Higher Yield and Concentrations of THCA than the Parental “PNW39” Cultivar

This Example demonstrates that hybrid cultivars of Cannabis sativa L. plants derived from crossing a resistant “PNW39” with a susceptible second parent are resistant to powdery mildew and have higher THCA concentrations than their “PNW39” parent.

“PNW39” flowers had an average of 20.96% THCA (wt/wt; n=5), and an average yield of 40.06 g/plant (n=302). Two cultivars of progeny of “PNW39” and a susceptible second parent were assayed for THCA content, yield, and resistance to powdery mildew. The first cultivar, “CB”, had an average of 27.43% THCA (wt/wt; n=9), and an average yield of 58.88 g/plant (n=768). The second cultivar, “WS”, had an average of 26.038% THCA (wt/wt; n=6) and an average yield of 47.84 g/plant (n=232). Both “CB” and “WS” were found to be resistant to infection by powdery mildew. These values are all represented in Table 3.

TABLE 3
Quantification of THCA and yield from
resistant parent and progeny cultivars.

| | PNW39 (parent) | CB (progeny) | WS (progeny) |
|---|---|---|---|
| THCA content | 20.96% | 27.43% | 26.038% |
| (wt/wt) | | | |
| Average yield | 40.06 | 58.88 | 47.84 |
| (g/plant) | | | |

### Example 6: Identification of Candidate Powdery Mildew Resistance Alleles

This Example demonstrates that multiple candidate alleles that result in powdery mildew resistance have been identified.

Transcriptomic analysis of nine powdery mildew-resistant cultivars was performed using flower tissue from material during the 7th week of the flowering cycle, with 3 replicates of each cultivar assayed on HiSeq4000, resulting in 30× genome coverage. At the time of sampling, plants were not under disease pressure from G. ambrosiae. Two assembly approaches were used: i) a genome-guided assembly using reads from all assayed cultivars (“the consensus”), and ii) de novo transcriptome assembly for each of the nine cultivars assayed. Blastn was used to search for the marker LH3804 in the consensus transcriptome sequence, in order to identify putative powdery mildew resistance genes. SEQ ID NOs: 1-28 were identified as putative transcripts from powdery mildew resistance genes, encoding polypeptides SEQ ID NOs: 29-56. Statistically significant blast results returned putative R genes belonging to two families: LRR-like proteins, or leucine-rich repeat-like proteins, and RPP8-like proteins, or RECOGNITION OF PERONOSPORA PARASITICA 8-like proteins, consistent with the location of LH3804 in the Cs10 genome. The identified sequences from the consensus were then blasted against the Cs10 genome to confirm their location on the tail end of chromosome 2 of the Cs10 genome.

### Example 7: Identification of Putative Powdery Mildew Resistance Alleles

This Example provides further analysis of candidate powdery mildew resistance alleles.

The nucleotide sequences SEQ ID NOs: 6 and 27 were translated into amino acid sequences SEQ ID NOs: 34 and 55 respectively using the ExPasy translation tool, and SEQ ID NOs: 34 and 55 were then analyzed using the InterProScan online protein domain classification tool.

SEQ ID NO: 6 encodes the 960 amino acid SEQ ID NO: 34, containing a C-terminal RX-like coiled coil domain, a central NB-ARC (nucleotide binding adaptor shared by APAF-1, R proteins, and CED-4 domain, and an N-terminal Leucine-rich-repeat (LRR) domain. These domains make up a classic structure that places the protein encoded by this transcript as a plant disease resistance protein in the NB-ARC-LRR protein family IPR044974. The predicted protein shows a high level of homology to the Arabidopsis resistance protein RPP8, which is known to function in the hypersensitive response to multiple biotrophic pathogens, including pathogens of the lifestyle employed by Golovinomyces ambrosiae.

SEQ ID NO: 27 encodes the 937 amino acid SEQ ID NO: 55, containing the same predicted domains as SEQ ID NO: 34 and belonging to the same protein family, IPR044974. BLAST searches using SEQ ID NO: 34 reveal a high level of homology to Arabidopsis disease resistance protein CAR1 (CEL Activated Resistance-1), which is known to function in a similar manner to RPP8 yet is a distinct gene.

Multiple other transcripts from SEQ ID NOs: 1-28 were found to be partial transcripts of either SEQ ID NO: 6 or SEQ ID NO: 27, or were expressed at levels much higher or lower than expected for an NB-ARC-LRR protein. As such, the genes of chromosome 2 that produce the transcripts of SEQ ID NOs: 6 and 27 were the primary candidates for PM1, genes for producing resistance to powdery mildew.

1. An isolated Cannabis sativa L. plant resistant to powdery mildew, wherein the plant comprises one or more powdery mildew resistance genes, and
wherein the plant produces flowers with greater than 22%, 23%, 24%, 25%, or 26% tetrahydrocannabinolic acid (THCA) (wt/wt).

2. (canceled)

3. The isolated plant of claim 1, wherein the resistance gene: is located on Chromosome 2 of GenBank assembly accession no. GCA_900626175.2.

4. The isolated plant of claim 1, wherein the resistance gene encodes a protein in the NB-ARC-LRR family.

5. (canceled)

6. (canceled)

7. The isolated plant of claim 1, wherein the resistance gene encodes a transcript comprising:
(i) a polynucleotide at least 75% identical, at least 80% identical, at least 85% identical, at least 90% identical, at least 95% identical, at least 96% identical, at least 97% identical, at least 98% identical, or at least 99% identical to a polynucleotide selected from the group consisting of SEQ ID NO: 6 and SEQ ID NO:27; or
(ii) SEQ ID NO: 6 or SEQ ID NO: 27.

8.-11. (canceled)

12. The isolated plant of claim 1, wherein the resistance gene encodes a protein comprising a polypeptide at least 75% identical, at least 80% identical, at least 85% identical, at least 90% identical, at least 95% identical, at least 96% identical, at least 97% identical, at least 98% identical, at least 99% identical, or 100% identical to a polypeptide selected from the group consisting of SEQ ID NO: 34 and SEQ ID NO: 55.

13.-15. (canceled)

16. The isolated plant of claim 1, wherein the plant comprises more than one resistance gene.

17. The isolated plant of claim 16, wherein the plant comprises two, three, or four resistance genes.

18. The isolated plant of claim 1, wherein the resistance gene allele is a dominant allele and/or wherein the plant is homozygous for the resistance gene allele.

19. (canceled)

20. The isolated plant of claim 18, wherein the resistance gene allele is a dominant allele and the plant is heterozygous for the resistance gene allele.

21. The isolated plant of claim 1, wherein the plant is resistant to infection by a fungus of the genus Golovinomyces.

22. The isolated plant of claim 21, wherein the plant is resistant to infection by fungi of the species Golovinomyces ambrosiae.

23. (canceled)

24. The isolated plant of claim 1, wherein the plant produces at least 41 g, at least 42 g, at least 43 g, at least 44 g, at least 45 g, at least 46 g, or at least 47 g per plant of harvestable floral material.

25. A plant part, tissue, or cell of the isolated plant of claim 1.

26. (canceled)

27. The isolated plant of claim 24, wherein the harvestable material comprises flowers and/or flower buds, leaves, stalks, and seeds.

28. The isolated plant of claim 1, wherein the plant has increased lateral branching as compared to a control Cannabis sativa L. plant, wherein the control Cannabis sativa L. plant is a naturally-occurring powdery mildew resistant plant.

29. The isolated plant of claim 1, wherein the plant has at least 5%, at least 10%, at least 15%, at least 20%, at least 25%, at least 30%, at least 35%, at least 40%, at least 45%, or at least 50% more top of the canopy flowers as compared to a control Cannabis sativa L. plant, wherein the control Cannabis sativa L. plant is a naturally-occurring powdery mildew resistant plant.

30. The isolated plant of claim 1, wherein the plant has increased flower site stacking as compared to a control Cannabis sativa L. plant, wherein the control Cannabis sativa L. plant is a naturally-occurring powdery mildew resistant plant.

31. (canceled)

32. The isolated plant of claim 28, wherein the control Cannabis sativa L. plant is from the ‘PNW39’ population of plants.

33. A method of selecting a Cannabis sativa L. plant resistant to powdery mildew, comprising:
a. crossing a first Cannabis sativa L. plant resistant to powdery mildew to a second Cannabis sativa L. plant to produce a population of offspring Cannabis sativa L. plants;
b. genotyping the offspring population of Cannabis sativa L. plants for the presence of one or more powdery mildew resistance genes; and
c. selecting an offspring Cannabis sativa L. plant on the basis of the genotyping of step (b),
wherein the selected offspring Cannabis sativa L. plant is resistant to powdery mildew.

34.-55. (canceled)

56. A plant selected by the method of claim 33.
